SEQUENCE LISTING (45 sequences)

SEQ ID NO: 1 - RNA, 67 nt, synthetic construct
  source 1..67: mol_type = other RNA
gggtcggcatggcatctccacctcctcgcggtccgacctgggctacttcggtaggctaagggagaag

SEQ ID NO: 2 - RNA, 15 nt, synthetic construct
  source 1..15: mol_type = other RNA
  modified_base 1: mod_base = OTHER; note = Monophosphate attached to the 5'-end
  modified_base 8: mod_base = p
cttggacntaacaaa

SEQ ID NO: 3 - DNA, 587 bp, synthetic construct
  source 1..587: mol_type = other DNA
taatacgactcactatagggtcactatgtagactggagaatgtggcgcgatgtgaagagacgaaaaatggcctatgaatacgcagatgagaggctacgtattaattcactcaggaagaataccattttgccaaaaattcttcaggatgtggctgatgaagaaattgctgccctcccccgggatagctgtcctgttagaatcagaaatcggtgtgttatgacgtcccgtccgcgtggtgtgaagcggcgctggaggcttagtcgtatagtcttccgtcacttagctgaccatgggcaactttctgggatccagcgagcgacatggtaaatgagctccagaacctattgagcttgcagggaagccaagcttgcagttccagcaagcaaagattttttttaatagaccaaaccctaatctctacaggggcccagtacagttgtttggcctacctgatgctatctctaaactacttttaaaatgaagacatttgggtgtttcatgtcagtgaattatcttttctgggtcggcatggcatctccacctcctcgcggtccgacctgggctacttcggtaggctaagggagaag

SEQ ID NO: 4 - DNA, 520 bp, synthetic construct
  source 1..520: mol_type = other DNA
taatacgactcactatagggcatactgttcccatacatagtacaccatgttgtataataaatattttctgtgaatgttttttataccctccttttaagtttaatcttttagtaacatataggggtttttgtattaagagcattatttcataaaatgctatcttatacataatccaactaattgacatgtagctcatgtatggcagtttgtaatcctggactagctttgctgacctgcagagctccccttaactattattgttagataaacagattggatagcaaggctgctgttctggattatctaatgctccacttcttcttgtgatggctgaacaaaagaaccactaagtgttttcttgtccatccttaagggactttaaaacaaattatgcctctttaagtccttgaaagtctccatctaaagaggaaaagtttgttggaacagtatatattccctatttttatgtttatttaatccataacagaaatgagtaagggtagcacaaagttctcattca

SEQ ID NO: 5 - DNA, 39 bp, synthetic construct
  source 1..39: mol_type = other DNA
aacagtatgccctttgttaagtccaagagaaaagataat

SEQ ID NO: 6 - DNA, 60 bp, synthetic construct
  source 1..60: mol_type = other DNA
cactgacatgaaacacccaaatgtcttcattttaaaagtagtttagagatagcatcaggt

SEQ ID NO: 7 - DNA, 60 bp, synthetic construct
  source 1..60: mol_type = other DNA
gagggtataaaaaacattcacagaaaatatttattatacaacatggtgtactatgtatgc

SEQ ID NO: 8 - RNA, 15 nt, synthetic construct
  source 1..15: mol_type = other RNA
  modified_base 1: mod_base = OTHER; note = Monophosphate attached to the 5'-end
  modified_base 8: mod_base = p
gcccggantgatggg

SEQ ID NO: 9 - DNA, 587 bp, synthetic construct
  source 1..587: mol_type = other DNA
taatacgactcactatagggtgatgatggccaaagagaagccaccgataactgtagttggagatgttggaggccgcatcgcaatcatcgtggatgacattattgacgatgtggagagttttgttgctgccgcggagatcctgaaagagagaggcgcctataagatctatgttatggccacccacggcatcctgtctgcagaggcccctcgcctgattgaggagtcctccgtagacgaggtggtggtgacgaatactgtccctcatgaggttcagaagctgcaatgtcccaagataaagactgtggatatcagtttgattctttctgaagccattcggagaatccacaatggagagtccatggcctaccttttccgaaacatcactgtggatgactagctttcacgagggtctcgaccctggacctcctgagggaaacatggaaaaagcagtgccatgagtgatacagtgtttccttgcaagggaggactcgaaacagcctggagttagatatcttcttttgggtcggcatggcatctccacctcctcgcggtccgacctgggctacttcggtaggctaagggagaag

SEQ ID NO: 10 - DNA, 520 bp, synthetic construct
  source 1..520: mol_type = other DNA
taatacgactcactataggggaggagggattaaaagagtcaggaccatgttgtagctaatggataaatatcataacatggccttacatgtctgctgtcatcagccctgttccttaaaagttctagctgctttcttaaaaataatctgaaaatcttattgatactaaagaggagttaaaggcacataaagtcttaactctataatgttcatttagttgtttcagctccagggaaatggaggtattgatgttgaacctggttagggaagctgagcgcctgtggccctattactatccagttggcctctcccaaatcaacttcaagtcttttatagagaatcgtatttttctttcagaaattgttatgcctacagccattgaaaaatgaagcattcatgttgttacatcttccaaggatgtcagattagaaaatagcatcccacctctgggtatctgagtggctctgaagttgcaaataaaataatttgttgtcttgctagtcttttcattagacagcagaag

SEQ ID NO: 11 - DNA, 39 bp, synthetic construct
  source 1..39: mol_type = other DNA
tccctcctcccccccatcaatccgggcaaaagaagatat

SEQ ID NO: 12 - DNA, 60 bp, synthetic construct
  source 1..60: mol_type = other DNA
taactccaggctgtttcgagtcctcccttgcaaggaaacactgtatcactcatggcactg

SEQ ID NO: 13 - DNA, 60 bp, synthetic construct
  source 1..60: mol_type = other DNA
acatgtaaggccatgttatgatatttatccattagctacaacatggtcctgactctttta

SEQ ID NO: 14 - RNA, 15 nt, synthetic construct
  source 1..15: mol_type = other RNA
  modified_base 1: mod_base = OTHER; note = Monophosphate group attached to the 5'-end
  modified_base 8: mod_base = p
tcagtgtncgaatca

SEQ ID NO: 15 - DNA, 587 bp, synthetic construct
  source 1..587: mol_type = other DNA
taatacgactcactatagggctatgttcttgtcgcctccgaccgggtggccgccagcaatattgtccagatgaaggacgatcatgacaagatgtttaagatgagtgaaaagatattactcctgtgtgttggagaggctggagacactgtacagtttgcagaatatattcagaaaaacgtgcaactttataagatgcgaaatggatatgaattgtctcccacggcagcagctaacttcacacgccgaaacctggctgactgtcttcggagtcggaccccatatcatgtgaacctcctcctggctggctatgatgagcatgaagggccagcgctgtattacatggactacctggcagccttggccaaggccccttttgcagcccacggctatggtgccttcctgactctcagtatcctcgaccgatactacacaccgactatctcacgtgagagggcagtggaactccttaggaaatgtctggaggagctccagaaacgcttcatcctgaatctgccaacctgggtcggcatggcatctccacctcctcgcggtccgacctgggctacttcggtaggctaagggagaag

SEQ ID NO: 16 - DNA, 520 bp, synthetic construct
  source 1..520: mol_type = other DNA
taatacgactcactatagggttgacaaaaatggcatccatgaccccatgttgtattccttccccaaacagggctcctaacatcatgtcctccctcccacttgccagggaacttttttttgatgggctcctttatttttttctactcttttcaggcgcactcttgataaatggttaattcagaataaaggtgactatggatataattgagccctctggtccaggtctcagtttacctaatattacctcagaaaggatatggagggaagatgatctttttgccaggtctgacttttcttcctgctccgccctccattaacgctcagtaccctttagcagctgacggccccacgttctactccatgcttggcttcctttccaactagctctttcatatattttacttgctagtatctccattctctctaaagtagtggttctttttgcccttaaacttaaatttttaaattaattaacctgaattaataatacatgcacttaatgtaacatgcaaacagtaca

SEQ ID NO: 17 - DNA, 39 bp, synthetic construct
  source 1..39: mol_type = other DNA
ttttgtcaaccctgattcgaacactgaaggttggcagat

SEQ ID NO: 18 - DNA, 60 bp, synthetic construct
  source 1..60: mol_type = other DNA
caggatgaagcgtttctggagctcctccagacatttcctaaggagttccactgccctctc

SEQ ID NO: 19 - DNA, 60 bp, synthetic construct
  source 1..60: mol_type = other DNA
aggacatgatgttaggagccctgtttggggaaggaatacaacatggggtcatggatgcca

SEQ ID NO: 20 - RNA, 15 nt, synthetic construct
  source 1..15: mol_type = other RNA
  modified_base 1: mod_base = OTHER; note = Monophosphate group attached to the 5'-end
  modified_base 8: mod_base = p
ggagctgnagtgtcc

SEQ ID NO: 21 - DNA, 587 bp, synthetic construct
  source 1..587: mol_type = other DNA
taatacgactcactatagggtgcgcatcgcggaagccctcagcaagatgaagctgcagcccttcgccacagaggcagatgtggaggaggccctgcggctcttccaagtgtccacgttggatgctgccttgtccggtaccctgtcaggggtggagggcttcaccagccaggaggaccaggagatgctgagccgcatcgagaagcagctcaagcgccgctttgccattggctcccaggtgtctgagcacagcatcatcaaggacttcaccaagcagaaatacccggagcacgccatccacaaggtgctgcagctcatgctgcggcgcggcgagatccagcatcgcatgcagcgcaaggttctctaccgcctcaagtgagtcgcgccgcctcactggactcatggactcgcccacgcctcgcccctcctgccgctgcctgccattgacaatgttgctgggacctctgcctccccactgcagccctcgaacttcccaggcaccctcctttctgccccagaggaagggtcggcatggcatctccacctcctcgcggtccgacctgggctacttcggtaggctaagggagaag

SEQ ID NO: 22 - DNA, 520 bp, synthetic construct
  source 1..520: mol_type = other DNA
taatacgactcactatagggtgctgcctctgggcgcccgcctctccatgttgtatgggaagtgtgcttttggcatccgttaataataaagccacggtgtgttcaggtatctgtgggtttgtgcactcggtgacctgtcacctccatcgtgccctcatggcagggtaagtgtgagggaacagggtctggaggcagactggccagggtttctgacttggatctgccactcagacttctgggtaagtcacgtgacttgagtgctccggtttcttcatctgtttagcggggctcataaagccactcccttgatgctgggaatccagtgagaccagggagtggggaagcgcttctcaaagtgtgtgattttgatccgcctcacctgttccccagtaacttattggggggctgcgagccgtggtgcggcggtcagactgcctggattcatggaccctctccatgccttagtttccttatctgtaagcaggctgatactagaacctgcttcacagggttgtgcttag

SEQ ID NO: 23 - DNA, 39 bp, synthetic construct
  source 1..39: mol_type = other DNA
gaggcagcacccggacactacagctccttcctctggggc

SEQ ID NO: 24 - DNA, 60 bp, synthetic construct
  source 1..60: mol_type = other DNA
gaaaggagggtgcctgggaagttcgagggctgcagtggggaggcagaggtcccagcaaca

SEQ ID NO: 25 - DNA, 60 bp, synthetic construct
  source 1..60: mol_type = other DNA
ctttattattaacggatgccaaaagcacacttcccatacaacatggagaggcgggcgccc

SEQ ID NO: 26 - RNA, 15 nt, synthetic construct
  source 1..15: mol_type = other RNA
  modified_base 1: mod_base = OTHER; note = Monophosphate group attached to the 5'-end
  modified_base 8: mod_base = p
ctgctttnttctgaa

SEQ ID NO: 27 - DNA, 587 bp, synthetic construct
  source 1..587: mol_type = other DNA
taatacgactcactatagggggatgcgttccattttcacacttcacagtcatgaaaactgagaagactgtcttcagcgtgaactaaagttcacaggcagatcactgatccagaacacttcaagaactcgtcaaacagctcgataagcctttttgactgtgtacatctgtaccgggaataacattcctaggctgaaatttccacaaagaatagaacctgtacccagttcttcaggctgatttccctgacctcttgggcatttgtatttgtagtaaagtattgcagagattcctaagtattttatagcagccatcaaaattggactttgtattgtttattcataaaagacacttggtaatagacttcagtgaactctgtatgaatgcagtagtgtgtgtgcaaaatccgcttcctgagcgtagggtgctgagctggcgctagggctcggttgtgaaatacagcgtagtcagcccttgcgctcagtgtagaaacccacgtctgtaaggtcggtcttcgtccatgggtcggcatggcatctccacctcctcgcggtccgacctgggctacttcggtaggctaagggagaag

SEQ ID NO: 28 - DNA, 125 bp, synthetic construct
  source 1..125: mol_type = other DNA
taatacgactcactatagggatacactaagagcagccacaaaactgtaacctcaaggaaaccataaagcttggagtgccttaatttttaaccagtttccaataaaacggtttactacctgatcga

SEQ ID NO: 29 - DNA, 39 bp, synthetic construct
  source 1..39: mol_type = other DNA
ttagtgtatcccttcagaaaaaagcagatggacgaagac

SEQ ID NO: 30 - DNA, 60 bp, synthetic construct
  source 1..60: mol_type = other DNA
gaccttacagacgtgggtttctacactgagcgcaagggctgactacgctgtatttcacaa

SEQ ID NO: 31 - DNA, 60 bp, synthetic construct
  source 1..60: mol_type = other DNA
ttaaaaattaaggcactccaagctttatggtttccttgaggttacagttttgtggctgct

SEQ ID NO: 32 - RNA, 10 nt, synthetic construct
  source 1..10: mol_type = other RNA
gcatcccggg

SEQ ID NO: 33 - skipped

SEQ ID NO: 34 - DNA, 30 bp, synthetic construct
  misc_feature 1..12: note = RNA RNA segment
  misc_feature 13..30: note = DNA DNA barcode sequence
  source 1..30: mol_type = other DNA
ggcttcttcttgctcttaggtagtaggttc

SEQ ID NO: 35 - DNA, 39 bp, synthetic construct
  source 1..39: mol_type = other DNA
gaggcgagcggtcaattttcctaagagcaagaagaagcc

SEQ ID NO: 36 - DNA, 54 bp, synthetic construct
  misc_feature 1..12: note = RNA polyA+RNA
  misc_feature 13..54: note = DNA DNA barcode
  source 1..54: mol_type = other DNA
aaaaaaaaaaaaaaaaaaaaaaaaggcttcttcttgctcttaggtagtaggttc

SEQ ID NO: 37 - DNA, 20 bp, synthetic construct
  source 1..20: mol_type = other DNA
ggcttcttcttgctcttagg

SEQ ID NO: 38 - DNA, 20 bp, synthetic construct
  source 1..20: mol_type = other DNA
gtgattctcgtctttctgcg

SEQ ID NO: 39 - DNA, 20 bp, synthetic construct
  source 1..20: mol_type = other DNA
gtacttttctctttgcgcgg

SEQ ID NO: 40 - DNA, 20 bp, synthetic construct
  source 1..20: mol_type = other DNA
ggtcttcgctcggtcttatt

SEQ ID NO: 41 - RNA, 21 nt, synthetic construct
  source 1..21: mol_type = other RNA
gaaggagctgtagtgtccggg

SEQ ID NO: 42 - RNA, 21 nt, synthetic construct
  source 1..21: mol_type = other RNA
tctcttggacttaacaaaggg

SEQ ID NO: 43 - RNA, 21 nt, synthetic construct
  source 1..21: mol_type = other RNA
ccttcagtgttcgaatcaggg

SEQ ID NO: 44 - RNA, 21 nt, synthetic construct
  source 1..21: mol_type = other RNA
tttgcccggattgatgggggg

SEQ ID NO: 45 - RNA, 21 nt, synthetic construct
  source 1..21: mol_type = other RNA
atctgcttttttctgaaggga

(19) United States
(12) Patent Application Publication
(10) Pub. No.: US 2025/0327121 A1
(43) Pub. Date: Oct. 23, 2025

(54) Methods of Preparing RNA Samples for Sequencing, Methods of Sequencing RNA, and Methods of Preparing RNA Molecules with Modified Nucleic Acids

(71) Applicant: Thomas Jefferson University, Philadephia, PA (US)

(72) Inventors: Ya-Ming Hou, Philadephia, PA (US); Howard Gamper, Philadephia, PA (US)

(21) Appl. No.: 18/869,971

(22) PCT Filed: May 26, 2023
(86) PCT No.: PCT/US2023/067546

§ 371 Date: Nov. 27, 2024

Related U.S. Application Data
(60) Provisional application No. 63/346,650, filed on May 27, 2022.
(60) Provisional application No. 63/433,180, filed on Dec. 16, 2022.

(51) Int. Cl. C12Q 1/6869; C12N 15/10; C12Q 1/6806; C12Q 1/6855

(52) U.S. Cl. CPC C12Q 1/6869; C12N 15/1003; C12Q 1/6806; C12Q 1/6855

(57) ABSTRACT

Disclosed herein are methods of preparing an RNA sample for sequencing. In certain embodiments, the method includes contacting an RNA molecule in the sample with an RNA-dependent RNA polymerase (RdRp) such that the RdRp extends the RNA molecule from the 3′ end of the RNA molecule using the RNA molecule as a template. Also disclosed herein are kits for preparing an RNA sample for sequencing according to certain methods, as well as methods of sequencing RNA molecules using the prepared sample. Also disclosed herein are methods of preparing an RNA molecule with a modified base. In certain embodiments, the method includes ligating a left-arm RNA segment, a middle RNA segment including the modified base, and a right-arm RNA segment in the presence of a DNA splint and DNA disruptors.

### CROSS-REFERENCE TO RELATED APPLICATIONS

The present application claims priority under 35 U.S.C. § 119(e) to U.S. Provisional Patent Application No. 63/346,650, filed May 27, 2022, and U.S. Provisional Patent Application No. 63/433,180, filed Dec. 16, 2022, both of which are incorporated herein by reference in its entirety.

### STATEMENT REGARDING FEDERALLY SPONSORED RESEARCH

This invention was made with government support under HG011120 awarded by the National Institutes of Health. The government has certain rights in the invention.

### SEQUENCE LISTING

The XML file named “205961-7087WO1(00340)_Seq Listing.xml” created on May 17, 2023, comprising 50.5 Kbytes, is hereby incorporated by reference in its entirety.

### BACKGROUND

New generations of RNA sequencing technologies, such as nanopore sequencing, allow for rapid and real-time analysis of large RNA molecules. Sequencing accuracy, however; remains less than satisfactory.

There is a need to develop new compositions and methods that improve accuracy of RNA sequencing technologies. The present invention addresses this need.

Existing methods of creating long RNA molecules (such as 1 kb or longer) with modified nucleic acids have less than desirable yield. There is a need to develop novel high yield methods of preparing long RNA molecules. The present invention addresses this need, as well.

### SUMMARY

In some aspects, the present invention is directed to the following embodiments:

### Method of Preparing an RNA Molecule

In some embodiments, the present invention is directed to a method of preparing an RNA molecule present in a composition for sequencing.

In some embodiments, the method includes contacting the RNA molecule with an RNA-dependent RNA polymerase (RdRp) in the composition.

In some embodiments, the RdRp extends the the 3′ end of the RNA molecule using the RNA molecule as a template.

In some embodiments, the RNA molecule comprises a hairpin structure at the 3′ end.

In some embodiments, the RdRp is an eukaryotic RdRp, an RdRp from a Birnaviridae family virus, an RdRp from a Bunyaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, or an RdRp from a Reoviridae family virus.

In some embodiments, the RdRp is 3D polymerase (3D^{pol}) from a poliovirus.

In some embodiments, the composition further comprises a nucleoside triphosphate.

In some embodiments, the composition further comprises a magnesium ion (Mg²⁺) or a manganese (II) ion (Mn²⁺).

In some embodiments, the RNA molecule is fully extended such that RdRp-driven replication reaches the 5′ end of the RNA molecule.

In some embodiments, the RNA molecule comprises a modified nucleotide, which is optionally pseudouridine.

In some embodiments, the length of the RNA molecule is about 1 kilobase (kb) or longer, such as about 1.5 kb or longer, about 2 kb or longer, about 2.5 kb or longer.

In some embodiments, the method further comprises attaching a barcoding sequence to the RNA molecule extended by the RdRp.

### Method of Sequencing RNA Molecule

In some embodiments, the present invention is directed to a method of sequencing an RNA molecule.

In some embodiments, the method includes preparing a first RNA composition according to the “Method of preparing an RNA molecule” section above.

In some embodiments, the method further includes sequencing the RNA molecule extended by the RdRp in the first RNA composition.

In some embodiments, the sequencing the RNA molecule extended by the RdRp comprises a direct RNA sequencing.

In some embodiments, the sequencing comprises nanopore sequencing.

In some embodiments, the RNA molecule comprises a modified nucleotide, which is optionally pseudouridine.

In some embodiments, the method further comprises comparing the sequencing results of the native portion of the extended RNA molecule and the sequencing results of extended portion of the extended RNA molecule to identify the modified nucleotide.

### Kit for Preparing an RNA Molecule for Sequencing

In some embodiments, the present invention is directed to a kit for preparing an RNA molecule present in a composition for sequencing.

In some embodiments, the kit comprises an RNA-dependent RNA polymerase (RdRp) capable of extending a 3′ end of an RNA molecule using the RNA molecule as a template.

In some embodiments, the kit further comprises a manual instructing that the RNA molecule be contacted with the RdRp before performing the sequencing.

In some embodiments, the RNA molecule comprises a hairpin structure at the 3′ end.

In some embodiments, the RdRp is an eukaryotic RdRp, an RdRp from a Birnaviridae family virus, an RdRp from a Bunyaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, or an RdRp from a Reoviridae family virus.

In some embodiments, the RdRp is 3D polymerase (3D^{pol}) from a poliovirus.

In some embodiments, the kit further comprises a nucleoside triphosphate.

In some embodiments, the kit comprising a magnesium ion (Mg²⁺) or a manganese (II) ion (Mn²⁺).

In some embodiments, the kit further comprises a barcoding nucleic acid molecule, and an enzyme for attaching the barcoding nucleic acid molecule to the RNA molecule extended by the RdRp.

In some embodiments, the enzyme for attaching the barcoding nucleic acid molecule to the RNA molecule extended by the RdRp comprises an RNA ligase, optionally a T4 RNA ligase 1, T4 RNA ligase 2, or a derivative thereof.

### Method of Preparing an RNA Molecule Having Modified Nucleic Acid

In some embodiments, the present invention is directed to a method of preparing an RNA molecule having a modified nucleic acid.

In some embodiments, the method comprises preparing a ligation mixture.

In some embodiments, the ligation mixture comprises: a left-arm RNA segment for forming a 5′-portion of the RNA molecule; a middle RNA segment comprising the modified nucleic acid for forming a middle portion of the RNA molecule; a right-arm RNA segment for forming a 3′-portion of the RNA molecule; and a DNA splint molecule complementary to the RNA molecule, wherein the DNA splint molecule overlaps with an entirety of the middle RNA segment, a 3′-end of the left-arm RNA segment, and a 5′-end of the right-arm RNA segment.

In some embodiments, the method further comprises ligating the left-arm RNA segment, the middle RNA segment, and the right-arm RNA segment to form the RNA molecule having the modified nucleic acid.

In some embodiments, the method further comprises preparing the left-arm RNA segment by in vitro transcription of a first DNA template.

In some embodiments, the first DNA template encodes a pre-left-arm RNA segment comprising the left-arm RNA segment and a cis-cleaving ribozyme to the 3′-end of the left-arm RNA segment.

In some embodiments, after the in vitro transcription of the first DNA template, the cis-cleaving ribozyme in the pre-left-arm RNA segment removes itself from the pre-left-arm RNA segment, thereby resulting in a left-arm RNA segment having a homogeneous 3′-end.

In some embodiments, preparing the left-arm RNA segment comprises contacting the pre-left-arm RNA segment with a first DNA disruptor, and allowing the cis-cleaving ribozyme to remove itself from the pre-left-arm RNA segment in the presence of the first DNA disruptor.

In some embodiments, the first DNA disruptor is a DNA molecule complementary to a 3′-portion of the left-arm RNA segment.

In some embodiments, preparing the left-arm RNA segment comprises subjecting a mixture comprising the pre-left-arm RNA segment and the first DNA disruptor to one or more cycles of heating and cooling.

In some embodiments, the cis-cleaving ribozyme comprises at least one selected from the group consisting of a Hepatitis delta virus (HDV) ribozyme or HDV-like self-cleaving ribozyme, a hammerhead ribozyme, hairpin ribozyme, a Varkud Satellite (VS) ribozyme, a glmS ribozyme, and a twister ribozyme.

In some embodiments, preparing the left-arm RNA segment by in vitro transcription of the first DNA template comprises using PNK to enzymatically treating the left-arm RNA segment to form a mature 3′-OH end in the left-arm RNA segment, optionally the enzymatic treatment of the left-arm RNA segment is with a polynucleotide kinase (PNK).

In some embodiments, preparing the left-arm RNA segment further comprises purifying the left-arm RNA segment from a reaction mixture for preparing the left-arm RNA segment, and wherein purifying the left-arm RNA segment comprises: subjecting the reaction mixture to an agarose gel electrophoresis; isolating an agarose gel section comprising the left-arm RNA segment from the agarose gel; and isolating the left-arm RNA segment from the isolated agarose gel section.

In some embodiments, a length of the left-arm RNA segment ranges from about 200 bases to about 3,500 bases.

In some embodiments, the middle RNA segment is chemically synthesized.

In some embodiments, a length of the middle RNA segment ranges from about 5 bases to about 100 bases.

In some embodiments, the modified nucleic acid of the middle RNA segment comprises a modified base, a modified sugar group and/or a modified backbone.

In some embodiments, the right-arm RNA segment is prepared from in vitro transcription using a second DNA template.

In some embodiments, a length of the right-arm RNA segment ranges from about 200 bases to about 3,500 bases.

In some embodiments, the ligation mixture further comprises a second DNA disruptor complementary with a 3′-portion of the left-arm RNA segment.

In some embodiments, the ligation mixture further comprises a third DNA disruptor complementary with a 5′-portion of the right-arm RNA segment.

In some embodiments, the second DNA disruptor and the first DNA disruptor are the same or different.

In some embodiments, ligating the left-arm RNA segment, the middle RNA segment, and the right-arm RNA segment comprises subjecting the ligation mixture to an RNA ligase.

In some embodiments, a ratio between a molarity of the second DNA disruptor and/or the third DNA disruptor to a molarity of the left-arm RNA segment, the middle RNA segment and/or the right-arm segment is about 10 or larger.

In some embodiments, a temperature for ligating the left-arm RNA segment, the middle RNA segment, and the right-arm RNA segment ranges from about 14° C. to about 25° C.

In some embodiments, the method further comprises, after the ligation reaction, purifying the RNA molecule from the ligation mixture.

In some embodiments, purifying the RNA molecule from the ligation mixture comprises: subjecting the ligation mixture to an agarose gel electrophoresis; isolating an agarose gel section from the agarose gel, wherein the agarose gel section comprises the RNA molecule; and purifying the RNA molecule from the agarose gel section.

In some embodiments, a length of the RNA molecule prepared by the method ranges from about 400 bases to about 6,000 bases.

In some embodiments, a yield of RNA molecule based on a molarity of the left-arm RNA segment, the middle RNA segment and/or the right-arm segment is about 20% or greater.

In some embodiments, the RNA molecule prepared by the method is substantially free of heterogeneity and mismatches around a ligation point between the left-arm RNA segment and the middle RNA segment, and a ligation point between the middle RNA segment and the right-arm RNA segment.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 illustrates certain aspects of the method of preparing an RNA sample for sequencing, in accordance with some embodiments.

FIG. 2 illustrates how the sample preparation method herein is able to improve the accuracy of RNA sequencing, such as nanopore RNA sequencing, in accordance with some embodiments.

FIG. 3 provides a brief description of the 3D^{pol} RNA-dependent RNA polymerase, one of the RNA-dependent RNA polymerases (RdRp) suitable for the sample preparation method herein, in accordance with some embodiments.

FIG. 4 demonstrates that 3D^{pol} has sufficient thermodynamic fidelity (i.e., the ability to discriminate correct from incorrect NTP), in accordance with some embodiments. (The RNA sequence shown in FIG. 4 is GCAUCCCGGG, SEQ ID NO:32).

FIGS. 5A-5D demonstrate that 3D^{pol} has sufficient fidelity in discriminating the bases A, m⁶A, and m¹A in the template opposite to a UTP as the incoming nucleotide, in accordance with some embodiments. (The RNA sequence shown in FIG. 5A is GCAXCCCGGG, SEQ ID NO:33, X=A, m6A, or m1A).

FIGS. 6A-6B demonstrate that 3D^{pol} has a specific discrimination profile in NTP incorporation when the base in the template is A or m⁶A, in accordance with some embodiments.

FIGS. 7A-7B demonstrate that 3D^{pol} is able to copy short RNA molecules by reading through stable structures and modified nucleotides, in accordance with some embodiments.

FIGS. 8A-8F demonstrate that 3D^{pol} can copy long RNA molecules with or without modified bases, in accordance with some embodiments. Specifically, a circular DNA plasmid encoding a curlcake DNA template (an RNA molecule having minimized RNA structure) (Liu et al. Nat Commun. 2019; 10: 4079; doi: 10.1038/s41467-019-11713-9) was transcribed using T7 RNA polymerase with various mixtures of NTPs, with or without modified bases (FIGS. 8A-8C). The produced curlcake RNAs were then extended using 3D^{pol} with natural NTPs. (FIG. 8D) All curlcake RNA molecules successfully produced by T7 polymerases were extended by 3D^{pol} polymerase (FIGS. 8E-8F).

FIGS. 9A-9C demonstrate that the RNA molecules extended by 3D^{pol} is compatible with existing RNA sequencing methods, such as the Nanopore sequencing methods, in accordance with some embodiments. It is worth noting that the ligase used in FIG. 9C, as well as some other figures, is not limited to the depicted T4RNL2-KO. T4RNL1, as well as other ligases are suitable for this reaction.

FIGS. 10A-10B: RNA molecules extended by 3D^{pol} can be attached with barcoding sequences, in accordance with some embodiments. FIG. 10A: adding a barcode to the 3′-adaptor of the extended RNA molecule. (The RNA/DNA hybrid barcode oligonucleotide shown in FIG. 10A is made up of two nucleotide strands: GGCUUCUUCUUGCTCTTAGGTAGTAGGTTC, SEQ ID NO:34, and GAGGCGAGCGGTCAATTTTCCTAAGAGCAAGAAGAAGCC, SEQ ID NO:35) FIG. 10B: Adding a barcode after the polyA sequence of the extended RNA molecule. (The RNA/DNA hybrid barcode oligonucleotide shown in FIG. 10B is made up of two nucleotide strands:

AAAAAAAAAAAAAAAAAAAAAAAAGGCTTCTTCTTGCTCTTAGGTAGTA
GGTTC, SEQ ID NO: 36,
and
GAGGCGAGCGGTCAATTTTCCTAAGAGCAAGAAGAAGCC, SEQ ID
NO: 35).

FIG. 11 illustrates non-limiting examples of barcoding sequences (Smith et al. Genome Res. (2020)). (BC1: GGCTTCTTCTTGCTCTTAGG, SEQ ID NO:37, BC2: GTGATTCTCGTCTTTCTGCG, SEQ ID NO:38, BC3: GTACTTTTCTCTTTGCGCGG, SEQ ID NO:39, BC4: GGTCTTCGCTCGGTCTTATT, SEQ ID NO:40).

FIGS. 12A-12D: Four methods of assembly to synthesize RNA containing a site-specific internal modification. FIG. 12A: Assembly of a short left-arm RNA with a short right-arm RNA, the latter of which has a 5′-terminal modification in a 2-part splint ligation. FIG. 12B: Assembly of a short left-arm and a short right-arm RNA with a modification-containing middle RNA in a 3-part splint ligation. FIG. 12C: Terminal 3′-extension of a long left-arm RNA with a modified nucleoside 3′,5′-bisphosphate, followed by removal of the 3′-phosphate by an alkaline phosphatase, and joining with a long right-arm RNA in a 2-part splint ligation. FIG. 12D: Assembly of a long left-arm RNA and a long right-arm RNA with a short middle RNA with the internal modification in a 3-part splint ligation, in the presence of both a left-arm and a right-arm DNA disruptor. Short RNA (less than a 100-mer) is shown as a straight black line, whereas long RNA (more than a 100-mer) is shown as a straight black line with double daggers. The modified nucleotide is shown as a cyan dot, the splint DNA is shown in red, and the DNA disruptors are shown in grey.

FIGS. 13A-13D: Scheme of the 3-part splint ligation, in accordance with some embodiments. FIGS. 13A-13B: The matured left-arm RNA (e.g., the ˜500-mer in FIG. 13B) is transcribed with a 5′-triphosphate and is processed by HDV and T4 PNK to produce a homogeneous 3′-OH, the matured right-arm RNA (e.g., the ˜500-mer in FIG. 13B) is transcribed with a 5′-monophosphate, while the middle RNA (e.g., the 15-mer in FIG. 13B) containing a site-specific internal W is chemically synthesized. The three RNAs are assembled on a DNA splint (e.g., the 39-mer in FIG. 13B), in the presence of a left-arm and a right-arm DNA disruptor (e.g., 60-mer each), for joining by T4 RNL2 to produce the full length RNA (e.g., the ˜1 kb RNA in FIG. 13B). FIGS. 13C-13D: The left-arm RNA is transcribed as a fusion with the HDV ribozyme (in green) to produce a transcription product (e.g., the 570-mer in FIG. 13D), in which HDV self-cleaves to release the left-arm RNA (e.g., the ˜500-mer in FIG. 13D) with a 2′,3′-cyclic phosphate. Treatment with T4 PNK generates the mature 3′-OH end. In some embodiments, the left-arm and the right-arm RNA are synthesized in the range of a 500-mer.

FIGS. 14A-14D: HDV processing of the transcribed left-arm RNA at the 3′-end, in accordance with some embodiments. FIG. 14A: Denaturing PAGE (6%) analysis of HDV cleavage of the transcribed left-arm RNA of MCM5 over the cycling number of a heat-cool process. The left panel was cleavage performed without the left-arm disruptor, while the right panel was cleavage performed with the disruptor, each showing separation of the transcribed (570-mer) from the cleaved RNA (503-mer). The fraction of cleavage was calculated as the band intensity of the 503-mer over the sum of band intensity of the 503-mer and 570-mer. FIG. 14B: Denaturing PAGE (6%) analysis of cleavage of several transcribed left-arm RNAs, each in three conditions. Conditions “a” denotes no separate incubation for HDV cleavage, “b” denotes HDV cleavage in the presence of the respective left-arm disruptor over 4 cycles of heat-cool, and “c” denotes HDV cleavage and T4 PNK treatment of the HDV-cleaved 3′-end. The fraction of processing by HDV and T4 PNK is shown at the bottom of each condition. FIG. 14C: Denaturing PAGE (6%) analysis of ligation of the T4 PNK-treated HDV-cleaved left-arm RNA (PSMB2) with a 15-mer RNA in a 2-part splint ligation reaction as a function of time of T4 PNK hydrolysis. FIG. 14D: Efficiency of ligation as measured from data in FIG. 14C over time.

FIGS. 15A-15D: Step-by-step assembly of the 1 kb PSMB2 RNA containing an internal ψ, in accordance with some embodiments. FIG. 15A: Denaturing PAGE (6%) analysis of the in vitro transcription products of the left-arm and right-arm RNA, showing that while the right-arm RNA migrated as a homogeneous 503-mer, the left-arm RNA migrated as a distribution of the 570-mer (86%) and 503-mer (14%). M: molecular weight markers; FL: full-length RNA (1 kb) made by in vitro transcription; LA: left-arm RNA; RA: right-arm RNA. FIG. 15B: Denaturing PAGE (6%) analysis of HDV processing of the transcribed left-arm RNA over 4 heat-cool cycles, followed by T4 PNK hydrolysis. FIG. 15C: Denaturing PAGE (6%) analysis of a 3-part splint ligation, yielding 35% as the ψ-containing full-length RNA (ψ-FL, 1 kb), 50% as the mixture of 2-part ligation products consisting of the left-arm and the right-arm each joined with the ψ-containing RNA [(LA-ψ)+(ψ-RA)](518-mer each), and 15% as the mixture of un-ligated left-arm and right-arm RNA (503-mer each). Denaturing PAGE (6%) gels in FIGS. 15A-15C were each stained by SYBR-Gold. FIG. 15D: Bioanalyzer (Agilent) capillary gel analysis of the 1 kb ψ-containing PSMB2 RNA purified from a 1.2% agarose gel.

FIGS. 16A-16C: Importance of a pair of proximal DNA disruptors for 3-part ligation. FIG. 16A: Denaturing PAGE (6%) analysis of a series of 3-part ligation reactions to assemble a 1 kb PSMB2 RNA. FIG. 16B: A bar graph showing the yield of each 3-part ligation reaction in (FIG. 16A), where errors are deviations from the average of three technical replicates. FIG. 16C: Graphic representation of the individual reactions. (a) A standard 3-part ligation reaction consisting of the ψ-containing middle RNA, the DNA splint (red), one proximal pair of DNA disruptors (grey) and one distal pair of DNA disruptors (orange), and both the left- and right-arm RNAs, where the left-arm RNA is processed by HDV and T4 PNK as shown by a filled green circle; (b) the reaction without DNA disruptors; (c) the reaction containing just the proximal pair of DNA disruptors; (d) the reaction containing just the distal pair of DNA disruptors; (e) the reaction as in (a) but the left-arm RNA is transcribed without the ribozyme for processing as shown in an open green circle; and (f) a 2-part ligation reaction joining the left- and right-arm RNAs using a different splint DNA. The efficiency of ligation is calculated as the fraction of the 1,021-mer over the sum of the 1,021-mer, the two 503-mers, and the two 518-mers. Note that the 503-mers and the 518-mers cannot be readily resolved in the gel.

FIGS. 17A-17B: Efficiencies of 2-part and 3-part joining in a 3-part splint ligation reaction, in accordance with some embodiments. FIG. 17A: Efficiency of ligation by 2-part joining of the left-arm or right-arm RNA with a 15-mer to synthesize the 518-mer ψ-RNA is shown in grey, while that by 3-part joining of all three RNAs to synthesize the 1 kb ψ-RNA is shown in purple. FIG. 17B: The quality of gel-purified ψ-containing long RNA by a capillary gel analysis. MRPS14 RNA, PRPSAP1 RNA, and the PSMB2 RNA, all of 1,021 nts, were assembled from a left-arm (503-mer), a right-arm (503-mer), and a 15-mer ψ-RNA. PTTG1IP RNA of 626 nts was assembled from a left-arm (503-mer), a right-arm (108-mer), and a 15-mer ψ-RNA; MCM5 RNA of 300 nts was assembled from a left-arm (141-mer), a right-arm (144-mer), and a 15-mer ψ-RNA; while MCM5 RNA of 500 nts was assembled from a left-arm (242-mer), a right-arm (243-mer), and a 15-mer ψ-RNA.

FIGS. 18A-18B: Context-dependent efficiencies of 3-part splint ligation, in accordance with some embodiments. FIG. 18A: Denaturing PAGE (6%) analysis of a series of 3-part ligation reactions, showing assembly of the 1,021-mer of four RNAs with varying efficiencies. In these reactions, the in vitro transcribed and processed left-arm RNA (503-mer), or the in vitro transcribed right-arm RNA (503-mer), can separately join the ψ-containing middle RNA (15-mer) in a 2-part ligation reaction to form the 518-mer. FIG. 18B: The efficiency of 3-part ligation of each reaction in FIG. 18A. The efficiency is calculated as the fraction of the band intensity of the 1,021-mer over the sum of the band intensity of the 1,021-mer and the 503/518-mers.

FIGS. 19A-19E: Nanopore sequencing across ligation junctions of ψ-mRNAs generated by 3-part splint ligation, in accordance with some embodiments. Sequencing reads of ψ-mRNA of (FIG. 19A, GAAGGAGCUGUAGUGUCCGGG, SEQ ID NO:41) MCM5, (FIG. 19B, UCUCUUGGACUUAACAAAGGG, SEQ ID NO:42) MRPS14, (FIG. 19C, CCUUCAGUGUUCGAAUCAGGG, SEQ ID NO:43) PSMB2, (FIG. 19D, UUUGCCCGGAUUGAUGGGGGG, SEQ ID NO:44) PRPSAP1, and (FIG. 19E, AUCUGCUUUUUUCUGAAGGGA, SEQ ID NO:45). PTTG1IP are shown by a representative snapshot from the integrated genome viewer (IGV) of aligned nanopore reads to the hg38 genome (GRCh38 p10) at previously annotated ψ sites. The sequence for each mRNA is shown below, where nucleotides A is shown in green, C in blue, G in brown, and U in red. Highlighted are miscalled bases of each mRNA, while grey indicates corrected called bases. The ligation junctions are marked by arrows, showing homogeneous and accurate sequences for each mRNA. The GGG immediately following the ligation site of the right-arm RNA is underlined, representing the initiation site of T7 transcription of the right-arm RNA. Except for the V-mRNA for PTTG1Ip, which was generated as a 600-mer, all others were generated as a 1 kb-mer.

FIGS. 20A-20B: Optimization of splint ligation, in accordance with some embodiments. Assembly of a kb-long PSMB2 mRNA by T4 RNL2-catalyzed ligation of a 500-mer left-arm RNA with a 500-mer right-arm RNA on a 12-mer DNA splint. FIG. 20A: Efficiency of ligation (%) as a function of the molar ratio of the left-arm DNA disruptor relative to the left-arm RNA. FIG. 20B: Efficiency of ligation (%) as a function of time achieved by T4 RNL2-catalyzed reaction at 16, 25, and 37° C. The condition of ligation was as described in the standard 3-part ligation reaction.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

The study described herein (“the present study”) proposes a novel strategy to improve accuracy of current generation RNA sequencing technologies. In one aspect, RNA-dependent RNA polymerase (RdRp), such as the RNA polymerase 3D^{pol} from polio virus, can replicate RNA molecules by extending the RNA molecules from the 3′ end using the rest of the RNA molecule as templates.

The products of this replication/extension are double-stranded hairpin RNA molecules, which contain two-fold redundancy of most of the sequence information. These products can then be sequenced by, for example, nanopore sequencing technology. Since sequencing of the extended RNA includes sequencing both the native strand and the newly added complement strand, two layers of sequence information can be obtained at once, thus improving accuracy of the sequencing process.

The present study further demonstrated that the present sequencing strategy is able to distinguish among nucleotides with modifications, such as but not limited to those of isomeric molecular mass such as uridine (U) vs. pseudouridine (ψ).

Accordingly, in some aspects, the present invention is directed to a method of preparing an RNA sample for sequencing.

In some aspects, the present invention is directed to a method of sequencing an RNA molecule.

In some aspects, the present invention is directed to a kit for preparing an RNA sample for sequencing.

Furthermore, the present study developed novel methods of synthesizing RNA molecules with modified nucleic acids, such as modified bases. The novel method allows the synthesis of long RNA molecules, such as longer than 1,000 nucleotides, that include modified nucleic acids at predetermined locations, with good yield. This is in contrast to existing methods which can only achieve short RNA lengths (less than 200 nucleotides) with poor yield (less than 1-2%).

Accordingly, in some embodiments, the present invention is directed to methods of preparing an RNA molecule, such as an RNA molecule having one or more modified nucleic acids.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term ““or”” is used to refer to a nonexclusive ““or”” unless otherwise indicated. The statement ““at least one of A and B”” or ““at least one of A or B”” has the same meaning as ““A, B, or A and B.””

““About”” as used herein when referring to a measurable value such as an amount, a temporal duration, and the like, is meant to encompass variations of 20% or ±10%, in certain embodiments ±5%, in certain embodiments ±1%, in certain embodiments ±0.1% from the specified value, as such variations are appropriate to perform the disclosed methods.

“Hybridize” as used herein refers to two full complementary or partially complementary single-stranded DNA or RNA molecules form a double-stranded molecule through base pairing. Two strands of DNA/RNA molecules are considered to hybridize with each other if the two strands are 90% or more complementary to each other, such as 92% or more complementary, 95% or more complementary, 98% or more complementary, 99% or more complementary, 99.5% or more complementary, or 100% complementary to each other.

### Method of Preparing RNA Samples for Sequencing

In some embodiments, the instant specification is directed to a method of preparing an RNA sample for sequencing.

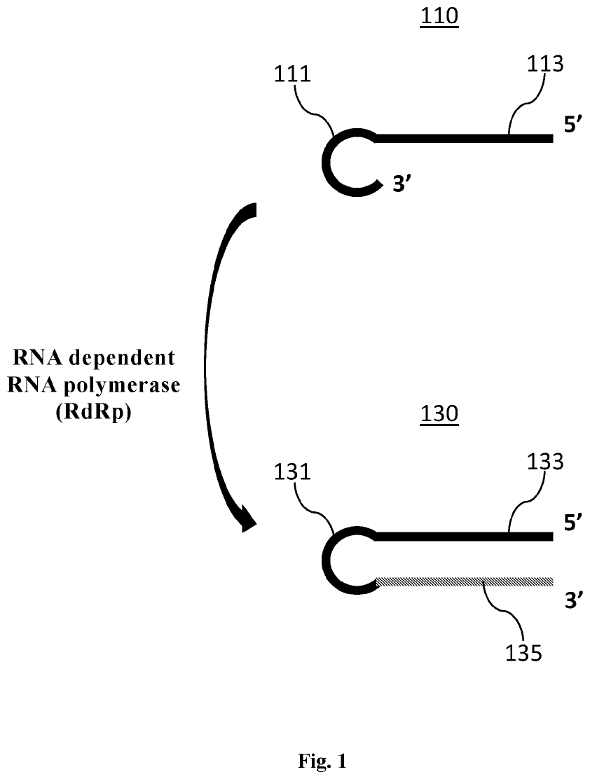

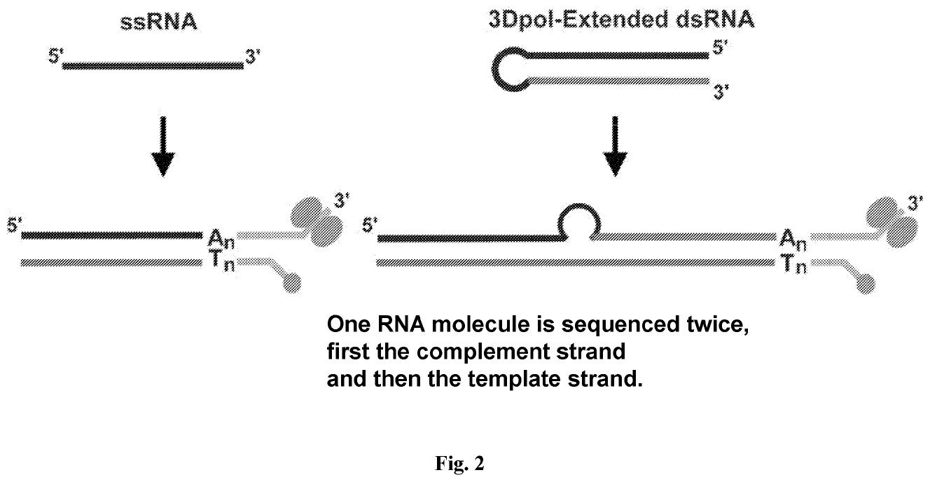

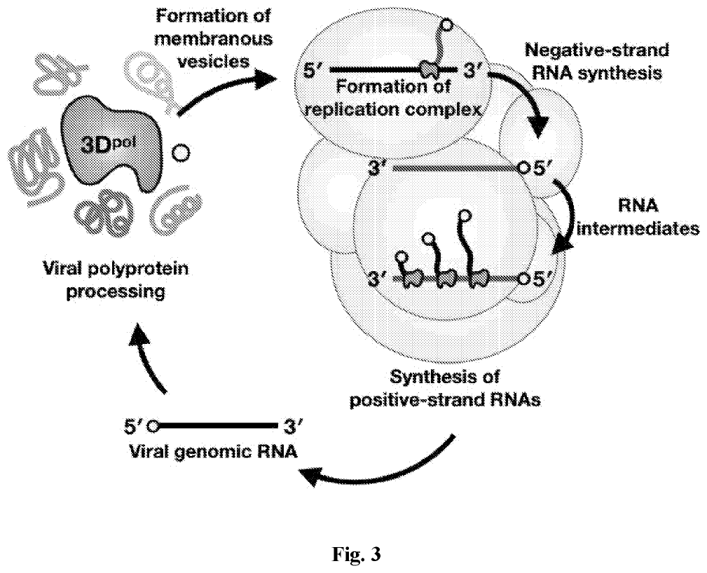

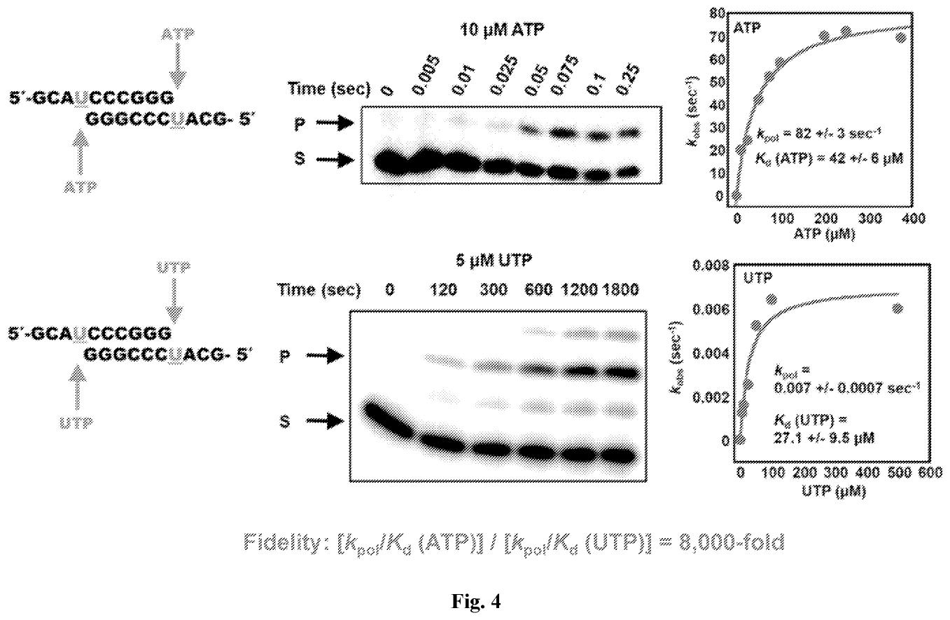

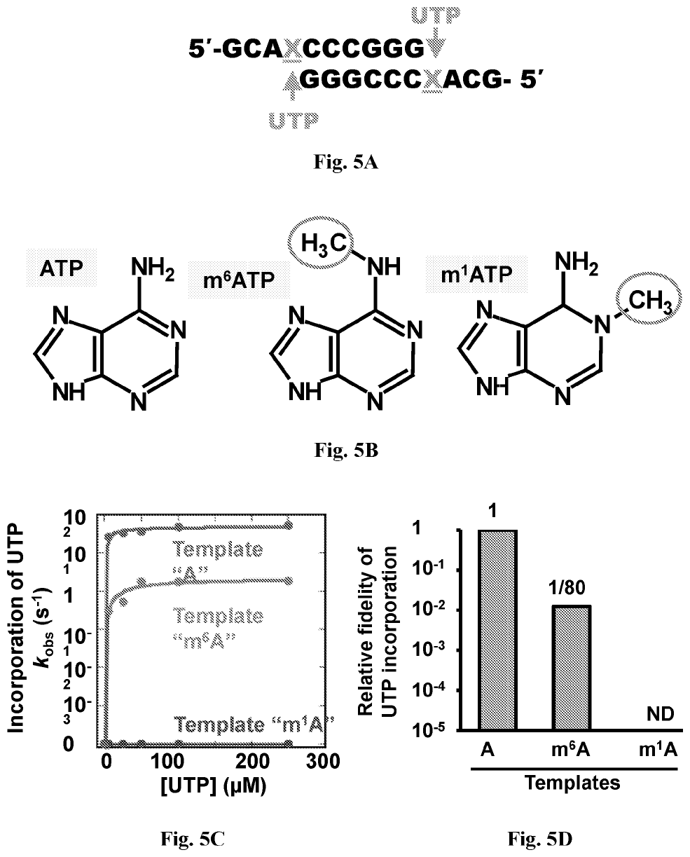

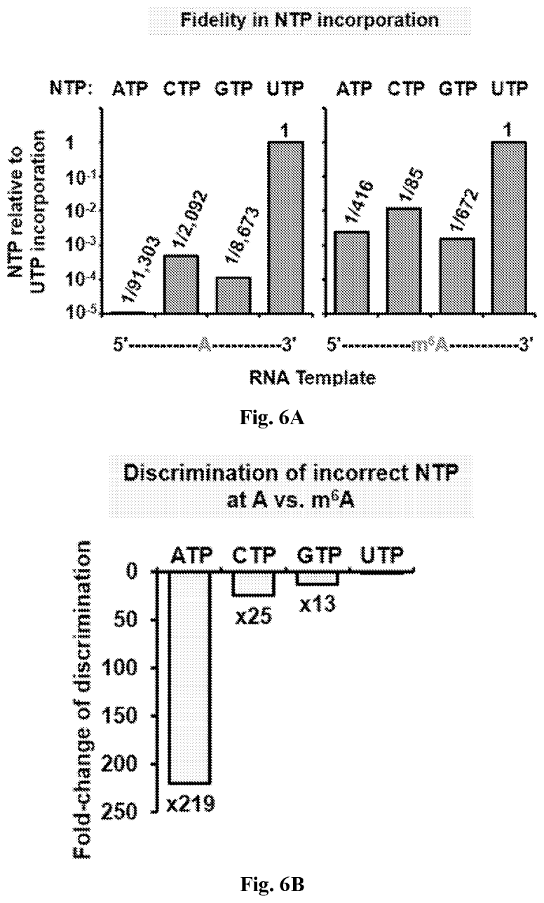

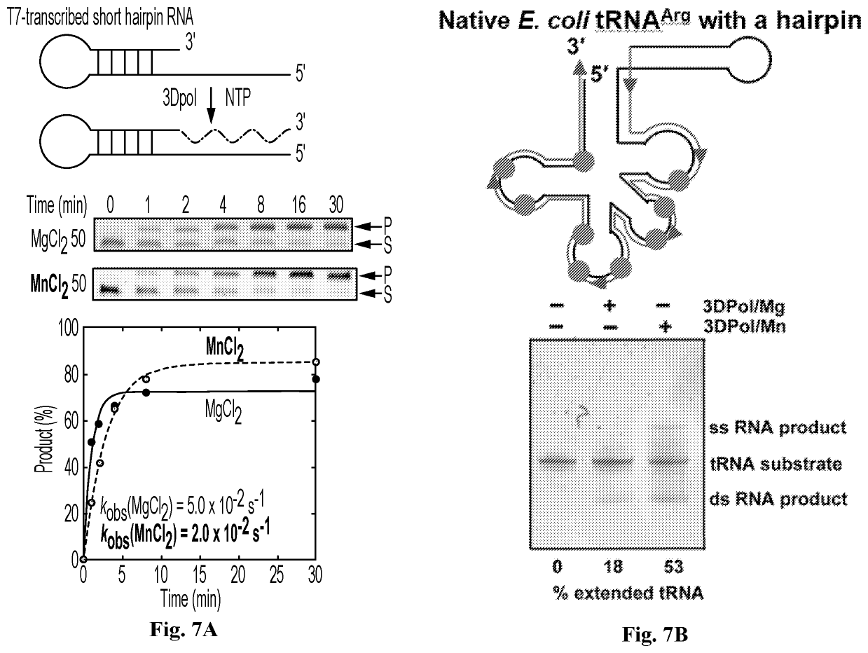

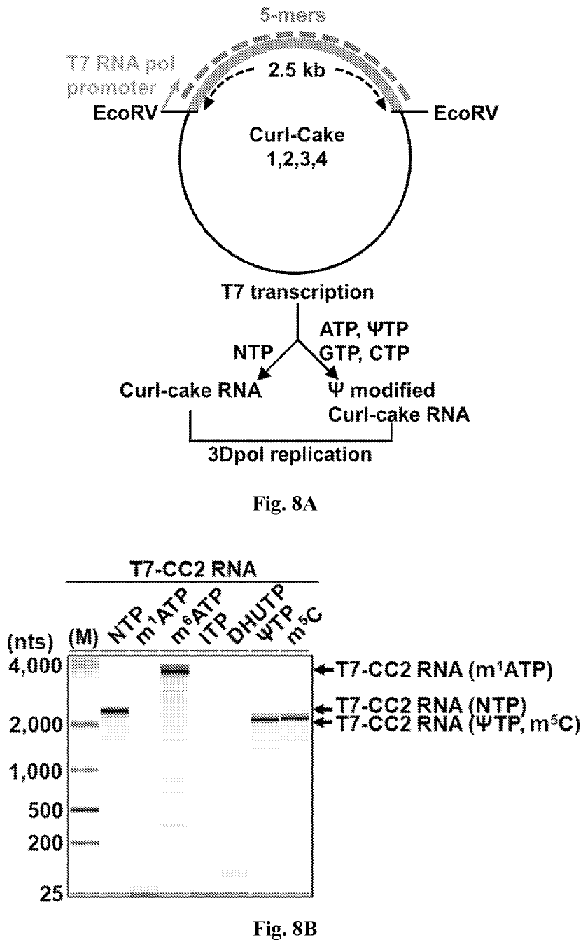

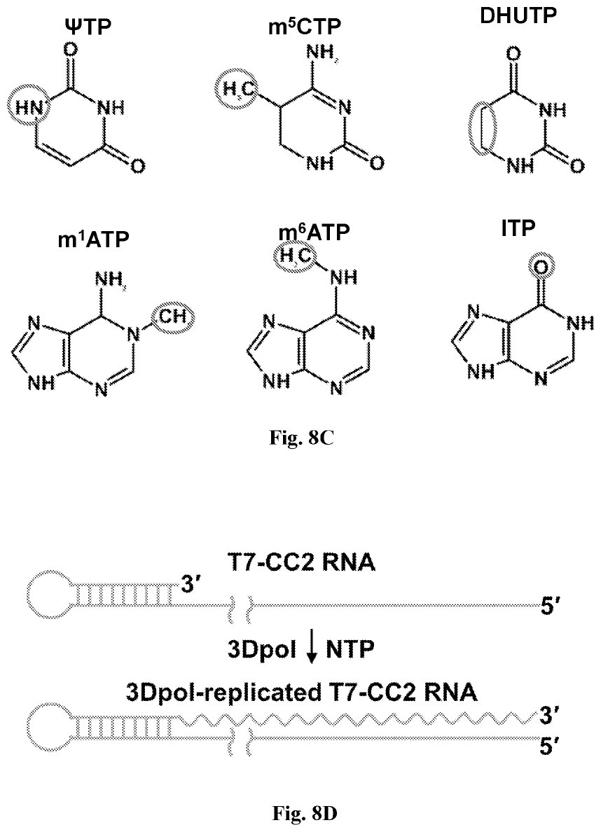

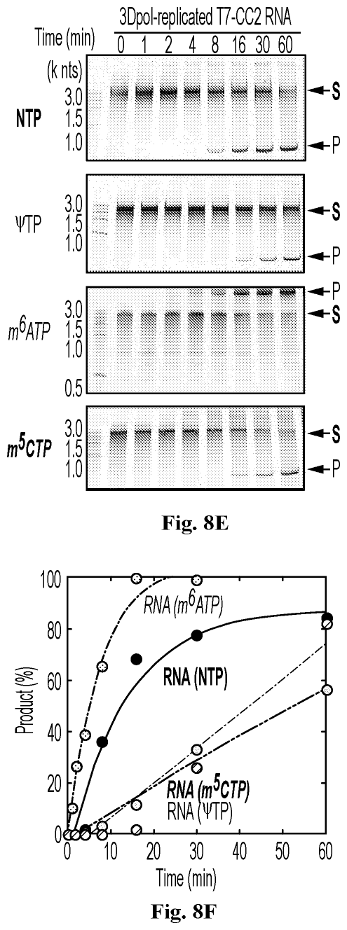

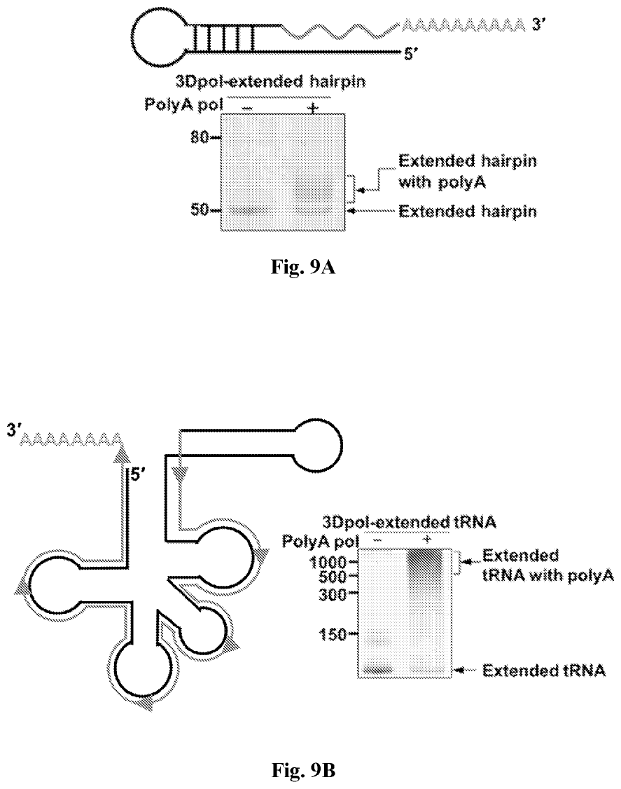

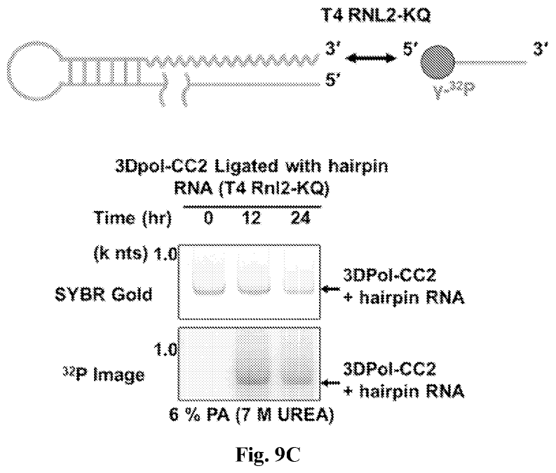

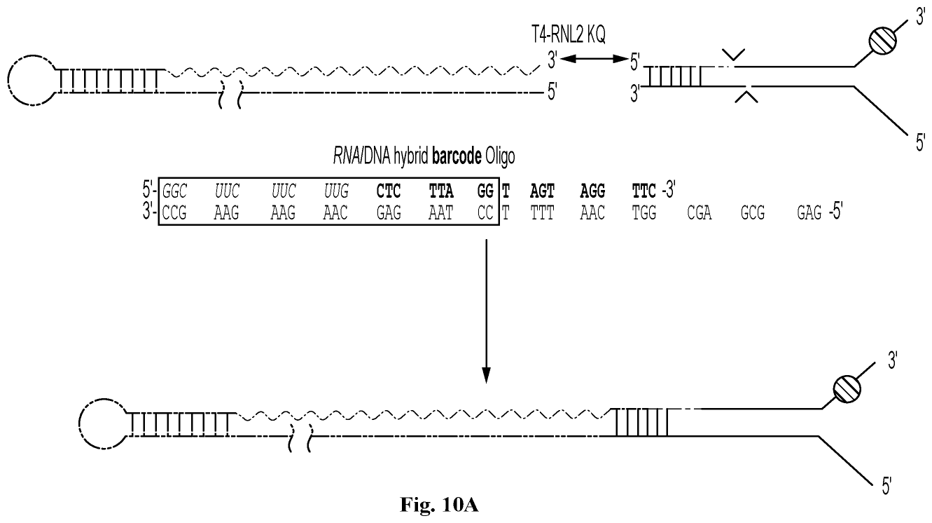

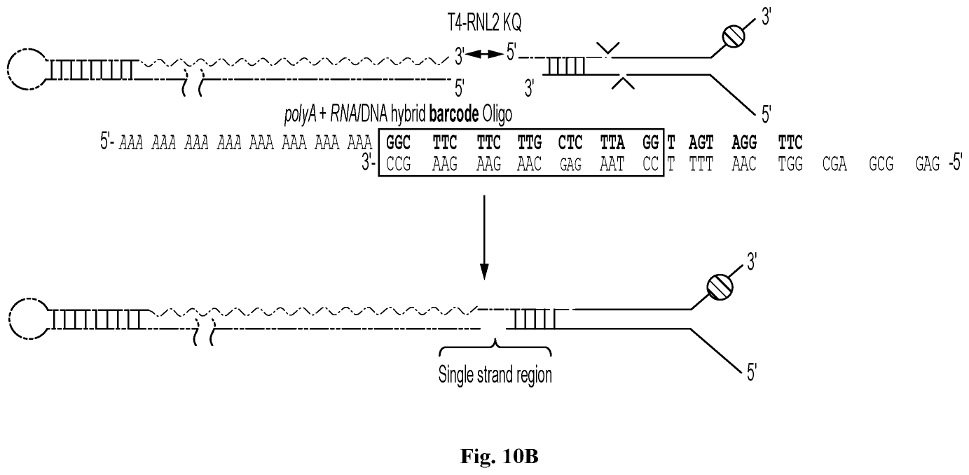

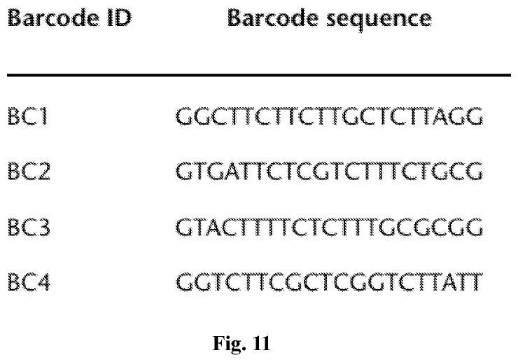

Referring to FIG. 1, in some embodiments, the method includes contacting an RNA molecule 110 in the sample with an RNA-dependent RNA polymerase (RdRp), wherein the RdRp extends the RNA molecule from the 3′ end of the RNA using the RNA molecule as a template.

In some embodiments, the RdRp extends the RNA molecule 110 from a hairpin structure 111 at the 3′ end of the RNA molecule. The structure 111 may comprise as few as 1-2 nucleotides. In some embodiments, the RdRp extends the RNA molecule 110 using the portion 113 that is not part of the 3′ hairpin structure as a template. The resulting extended RNA molecule 130 includes the hairpin structure 131, the native portion 133 and the extended portion 135 which is complementary to and sometimes hybridized to the native portion 133.

In some embodiments, the RdRp is an enzyme expressed in eukaryotic cells, such as an RdRp from a Birnaviridae family virus, an RdRp from a Bunyaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, an RdRp from a Reoviridae family virus, or combinations thereof.

In some embodiments, the RdRp is poliovirus 3D^{pol}, food-and mouth disease virus (FMDV) 3D^{pol}, ebola virus RdRp, yellow fever virus Yfpol, hepatitis C virus HCV RdRp, west Niles virus WNV RdRp, influenza A virus RdRp, Middle East Respiratory syndrome coronavirus (MERS-CoV) RdRp, SARS-CoV-2 RdRp, or combinations thereof.

In some embodiments, the RdRp is 3D polymerase (3D^{pol}) from a poliovirus.

In some embodiments, the sample further comprises a nucleoside triphosphate (NTP). In some embodiments, the NTP includes ATP, CTP, GTP, and/or UTP. In some embodiments, the NTP includes a modified NTP. In some embodiments, the NTP includes three natural NTPs and the fourth natural NTP is replaced with a modified NTP. For example, the NTP can include ATP, CTP, GTP (which are three natural NTPs), and WTP (the fourth natural NTP, UTP, is replaced with WTP), or include CTP, GTP, UTP (which are three natural NTPs) and m1ATP (the fourth natural NTP, ATP, is replaced with m1ATP). In some embodiments, two or more of the natural NTPs are replaced with corresponding modified NTPs. For each of the natural NTPs, one ordinary skill in the art would know which modified NTP can be used as replacement in RNA extension/replication. In some embodiments, the modified NTPs are incorporated to generate RNA standards for machine learning.

In some embodiments, the sample further comprises a magnesium ion (Mg²⁺) or a manganese (II) ion (Mn²⁺).

Referring to FIG. 1, in some embodiments, the RNA molecule 130 is fully extended such that the replication by RdRp reaches the 5′ end of the RNA.

In some embodiments, the RNA molecule 110 to be sequenced comprises a modified nucleotide. In some embodiments, the modified nucleotide includes a VTP.

In some embodiments, the length of the RNA molecule 110 is 1 kb or longer, such as about 1.5 kb or longer, about 2 kb or longer or about 2.5 kb or longer.

In some embodiments, the method further includes attaching a barcoding sequence to the RNA molecule that has been extended by the RdRp. In some embodiments, the enzyme for attaching the barcoding nucleic acid molecule to the RNA molecule extended by the RdRp includes an RNA ligase, optionally a T4 RNA ligase 1 or ligase 2, optionally a recombinant variant thereof.

### Method of Sequencing RNA Molecules

In some embodiments, the instant specification is directed to a method of sequencing an RNA molecule.

In some embodiments, the method includes preparing a first RNA sample using the method; and sequencing the RNA molecule extended by the RdRp in the prepared RNA sample.

In some embodiments, the first RNA sample is prepared according to the methods described herein, such as those detailed in the “Method of Preparing RNA Sample for Sequencing” section.

In some embodiments, the sequencing step is a direct RNA sequencing in which the sequence of the RNA is detected directly.

In some embodiments, extended RNA molecule is sequenced by a nanopore sequencing. The nanopore sequencing technology has been known for more than three decades and is well known in the art (Dream et al., Nature Biotechnology volume 34, pages 518-524 (2016)). The sequencing technology is described in, for example, Wang et al. (Nature Biotechnology volume 39, pages 1348-1365 (2021)). The entireties of the references are hereby incorporated herein by reference.

In some embodiments, the RNA molecule comprises a modified nucleotide, such as m1A, m6A, m5C, pseudouridine, dihydrouridine, m7G, and 2′-O-methylated nucleotide. In some embodiments, the modified nucleotide comprises a pseudouridine.

Referring to FIG. 1, in some embodiments, the method further comprises comparing the sequencing results of the native portion 133 of the extended RNA molecule 130 with the sequencing results of the extended portion 135 of the extended RNA molecule 130 to identify the modified nucleotide. As detailed elsewhere herein, current RNA sequence technologies often misidentify modified nucleotides and their adjacent nucleotides. Cross-referencing the sequencing results of portion 133 and portion 135 allows the correct identification.

### Kit for Preparing RNA Samples for Sequencing

In some aspects, the present invention is directed to a kit for preparing an RNA sample for sequencing.

In some embodiments, the kit is for performing the methods described herein, such as those detailed in the “Method of Preparing RNA Sample for Sequencing” section and “Method of Sequencing RNA molecule” section.

In some embodiments, the method includes an RNA-dependent RNA polymerase (RdRp) capable of extending RNA molecules from the 3′ end of the RNA molecules as a template; and a manual instructing that an RNA molecule to be sequenced be contacted with the RdRp before performing the sequencing to prepare a first sample.

In some embodiments, the RNA-dependent RNA polymerase extends the RNA molecule from the 3′ end of the single strand RNA.

In some embodiments, the RdRp is an eukaryotic RdRp, an RdRp from a Birnaviridae family virus, an RdRp from a Bunyaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, an RdRp from a Reoviridae family virus, or combinations thereof.

In some embodiments, the RdRp is poliovirus 3D^{pol}, food-and mouth disease virus (FMDV) 3D^{pol}, ebola virus RdRp, yellow fever virus Yfpol, hepatitis C virus HCV RdRp, west Niles virus WNV RdRp, influenza A virus RdRp, Middle East Respiratory syndrome coronavirus (MERS-CoV) RdRp, SARS-CoV-2 RdRp, or combinations thereof.

In some embodiments, the RdRp is 3D polymerase (3D^{pol} L) from a poliovirus.

In some embodiments, the kit further comprising a nucleoside triphosphate, such as ATP, CTP, GTP and UTP. In some embodiments, the kit further comprising a magnesium ion (Mg²⁺) or a manganese (II) ion (Mn²⁺). In some embodiments, the nucleoside triphosphate and/or the Mg²⁺ or Mn²⁺ ions are prepared in a mixture, such as an aqueous mixture, a solution or an aqueous solution.

In some embodiments, the kit further includes a barcoding sequence, as well as an enzyme for attaching the barcoding sequence to the RNA molecule extended by the RdRp. In some embodiments, the enzyme for attaching the barcoding nucleic acid molecule to the RNA molecule extended by the RdRp includes an RNA ligase.

### Method of Preparing RNA Molecules Having Modified Nucleic Acid

The present study developed novel methods of synthesizing RNA molecules with modified nucleic acids, such as modified bases.

Accordingly, in some embodiments, the present invention is directed to a method of preparing an RNA molecule, such as an RNA molecule having one or more modified nucleic acids.

In some embodiments, the method includes:
preparing a ligation mixture including:
   a left-arm RNA segment for forming a 5′-portion of the RNA molecule;
   a middle RNA segment comprising the modified nucleic acid for forming a middle portion of the RNA;
   a right-arm RNA segment for forming a 3′-portion of the RNA molecule; and
   a DNA splint molecule complementary to the RNA molecule and overlaps with an entirety of the middle RNA segment, a 3′-end of the left-arm RNA segment, and a 5′-end of the right-arm RNA segment; and
ligating the left-arm RNA segment, the middle RNA segment, and the right-arm RNA segment to form the RNA molecule having the modified nucleic acid.

### Left-Arm RNA Segment and Preparation Thereof

In some embodiments, the method further comprises preparing the left-arm RNA segment from in vitro transcription using a first DNA template.

RNA molecules prepared by in vitro transcription have 3′-end sequence heterogeneity, which together substantially reduces the yield of ligations. Accordingly, in some embodiments, the first DNA template encodes a pre-left-arm RNA segment comprising the sequence of the left-arm RNA segment and the sequence of a cis-cleaving ribozyme to the 3′-end of the left-arm RNA segment. In some embodiments, after the in vitro transcription of the first DNA template, the cis-cleaving ribozyme in the pre-left-arm RNA segment removes itself from the pre-left-arm RNA segment. Since many cis-cleaving ribozyme removes themselves from RNA sequences and leave homogenous 3′-ends in the remaining RNA molecules, the inclusion of the cis-cleaving ribozyme can significantly improve the yield of the ligation reactions.

RNA molecules, especially long RNA molecules, have structural heterogeneity which hinders the cis-cleavage reaction. DNA disruptors (i.e., DNA molecules that hybridize to RNA molecules and confer structure stability to the RNA molecule) proximal to the cleavage sites are able to reduce the structural heterogeneity and improves the efficiency of the cleavage reaction.

Accordingly, in some embodiments, preparing the left-arm RNA segment includes contacting the pre-left-arm RNA segment with a first DNA disruptor, and allowing the cis-cleaving ribozyme to remove itself from the pre-left-arm RNA segment in the presence of the first DNA disruptor.

In some embodiments, the first DNA disruptor is a DNA molecule complementary to a 3′-portion of the left-arm RNA segment.

In some embodiments, a length of the first DNA disruptor ranges from about 20 bases to about 100 bases, such as from about 30 bases to about 90 bases, from about 40 bases to about 80 bases, or from about 50 bases to about 70 bases. In some embodiments, the length of the first DNA disruptor is about 20 bases, about 30 bases, about 40 bases, about 50 bases, about 60 bases, about 70 bases, about 80 bases, about 90 bases, about 100 bases, or any ranges therebetween.

In some embodiments, a degree of complementarity between the first DNA disruptor and the left-arm RNA segment is about 90% or more, such as about 92% or more, about 95% or more, about 98% or more, about 99% or more, or 100%.

In some embodiments, the 3′-end (using the RNA strand as a reference) of the section formed by the first DNA disruptor hybridizing with the left-arm RNA segment is about 50 bases or less, such as about 40 bases or less, about 30 bases or less, about 20 bases or less, about 10 bases or less, or about 5 bases or less, from the 3′-end of the left-arm RNA segment.

In some embodiments, preparing the left-arm RNA segment comprises subjecting a mixture including the pre-left-arm RNA segment and the first DNA disruptor to one or more heat-cool cycles. In some embodiments, the mixture is subjected to 1 heat-cool cycle, 2 heat-cool cycles, 3 heat-cool cycles, 4 heat-cool cycles, 5 heat-cool cycles, 6 heat-cool cycles, 7 heat-cool cycles, 8 heat-cool cycles, 9 heat-cool cycles, 10 heat-cool cycles, or any ranges therebetween. In some embodiments, in each of the heat-cool cycles, the mixture is subjected to a temperature of 60° C. or higher, and then subjected to a temperature of 16° C. or lower.

In some embodiments, the cis-cleaving ribozyme includes at least one selected from the group consisting of a Hepatitis delta virus (HDV) ribozyme or HDV-like self-cleaving ribozyme, a hammerhead ribozyme, hairpin ribozymes, a Varkud Satellite (VS) ribozyme, a glmS ribozyme, and a twister ribozyme. Hepatitis delta virus (HDV) ribozyme and HDV-like self-cleaving ribozymes are described in, for example Webb et al. (RNA Biol. 2011 September-October; 8(5): 719-727). Hepatitis delta virus (HDV) ribozyme, HDV-like self-cleaving ribozymes, hammerhead ribozymes, hairpin ribozymes, Varkud Satellite (VS) ribozymes, and glmS ribozymes are described in, for example, Ferré-D'Amard et al (Cold Spring Harb Perspect Biol. 2010 October; 2(10): a003574). Twister ribozymes are described in, for example, Roth et al. (Nat Chem Biol. 2014 January; 10(1): 56-60).

Cis-cleaving ribozymes sometimes do not leave a mature 3′-OH end in the remaining RNA molecules. Accordingly, in some embodiments, preparing the left-arm RNA segment from in vitro transcription using the first DNA template includes enzymatically treating the processed left-arm RNA segment to form a mature 3′-OH end in the left-arm RNA segment. In some embodiments, the enzymatic treatment includes treating the processed left-arm RNA segment with a T4 polynucleotide kinase (PNK).

In some embodiments, a length of the left-arm RNA segment ranges from about 200 bases to about 3,500 bases, such as from about 300 bases to about 3,200 bases, or about 500 bases to about 3,000 bases.

In some embodiments, preparing the left-arm RNA segment further comprises purifying the left-arm RNA segment after the ribozyme cleavage reaction mixture. Purifying the left-arm RNA segment includes: subjecting the ribozyme cleavage reaction mixture to an agarose gel electrophoresis; isolating an agarose gel section containing a band corresponding to the left-arm RNA segment from the agarose gel; and isolating the left-arm RNA segment from the isolated agarose gel section.

### Middle RNA Segment

One of ordinary skills in the art would understand that, chemical synthesis is reliable for incorporating modified nucleic acid into an RNA molecule. Accordingly, in some embodiments, the middle RNA segment is chemically synthesized. In some embodiments, the middle RNA segment is synthesized using a solid-phase method.

In some embodiments, a length of the middle RNA segment ranges from about 5 bases to about 100 bases, such as from about 6 bases to about 90 bases, from about 7 bases to about 80 bases, from about 8 bases to about 70 bases, from about 9 bases to about 60 bases, from about 10 bases to about 50 bases, or from about 11 bases to about 40 bases, or from about 12 bases to about 30 bases.

In some embodiments, the modified nucleic acid of the middle RNA segment comprises a modified base, a modified sugar group and/or a modified backbone.

Non-limiting examples of modified bases include pseudouridine (Ψ), N1-methylpseudouridine (m1Ψ), 5-methylcytosine (m5C), deoxyuridine (dU), N1-methyladenosine (m1A), N6-methyladenosine (m6A), inosine (I), dihydrouridine (DHU) or the like.

Non-limiting examples of modified sugar group includes the sugar group of 2′-fluoro (2′F) RNA; the sugar group 2′-O-methyl (2′OMe) RNA; the sugar group locked nucleic acid (LNA); the sugar group of 2′-fluoro arabinose nucleic acid (FANA); the sugar group of hexitol nucleic acid (HNA); the sugar group of 2′-O-methoxyethyl (2′MOE), or the like.

Non-limiting examples of backbone modifications include phosphorothioate (PS) Modification, Boranophosphate Modification, or the Like.

### Right-Arm RNA Segment and Preparation Thereof

In some embodiments, the right-arm RNA segment is prepared from in vitro transcription using a second DNA template.

In some embodiments, a length of the right-arm RNA segment ranges from about 200 bases to about 3,500 bases, such as from about 300 bases to about 3,200 bases, or about 500 bases to about 3,000 bases.

In some embodiments a 5′-end of the right-arm RNA segment is a p-G (guanosine monophosphate). In some embodiments, the second DNA template is transcribed in the presence of GMP, in addition to NTP.

### DNA Disruptors for Aiding Ligation

RNA molecules, especially long RNA molecules, have structural heterogeneity which also hinders splint ligation reactions. DNA disruptors proximal to the ligation sites are able to reduce the structural heterogeneity and improves the ligation yield.

Accordingly, in some embodiments, the ligation mixture further includes: a second DNA disruptor complementary with a 3′-portion of the left-arm RNA segment; and a third DNA disruptor complementary with a 5′-portion of the right-arm RNA segment.

In some embodiments, the second DNA disruptor and the first DNA disruptor are the same or different.

In some embodiments, the second DNA disruptor is a DNA molecule complementary to a 3′-portion of the left-arm RNA segment. In some embodiments, the third DNA disruptor is a DNA molecule complementary to a 5′-portion of the right-arm RNA segment.

In some embodiments, a length of the second DNA disruptor and/or a length of the third DNA disruptor ranges from about 20 bases to about 100 bases, such as from about 30 bases to about 90 bases, from about 40 bases to about 80 bases, or from about 50 bases to about 70 bases. In some embodiments, the length of the first DNA disruptor is about 20 bases, about 30 bases, about 40 bases, about 50 bases, about 60 bases, about 70 bases, about 80 bases, about 90 bases, about 100 bases, or any ranges therebetween.

In some embodiments, a degree of complementarity between the second DNA disruptor and the left-arm RNA segment, and/or a degree of complementarity between the third DNA disruptor and the right-arm RNA segment is about 90% or more, such as about 92% or more, about 95% or more, about 98% or more, about 99% or more, or 100%.

In some embodiments, the 3′-end (using the RNA strand as a reference) of the section formed by the second DNA disruptor hybridizing with the left-arm RNA segment is about 50 bases or less, such as about 40 bases or less, about 30 bases or less, about 20 bases or less, about 10 bases or less, or about 5 bases or less, from the 3′-end of the left-arm RNA segment.

In some embodiments, the 5′-end (using the RNA strand as a reference) of the section formed by the third DNA disruptor hybridizing with the right-arm RNA segment is about 50 bases or less, such as about 40 bases or less, about 30 bases or less, about 20 bases or less, about 10 bases or less, or about 5 bases or less, from the 5′-end of the right-arm RNA segment.

### Ligation Reaction

In some embodiments, ligating the left-arm RNA segment, the middle RNA segment, and the right-arm RNA segment includes subjecting the ligation mixture to an RNA ligase. In some embodiments, the RNA ligase includes T4 RNA ligase 2 (RNL2) or a variant of RNL2.

In some embodiments, a ratio between a molarity of the second DNA disruptor and/or the third DNA disruptor to a molarity of the left-arm RNA segment, the middle RNA segment and/or the right-arm segment is about 10 or larger, such as about 12 or larger, about 15 or larger, about 20 or larger, about 30 or larger, about 40 or larger, or about 50 or larger.

In some embodiments, a temperature for ligating the left-arm RNA segment, the middle RNA segment, and the right-arm RNA segment ranges from about 14° C. to about 40° C., such as from about 16° C. to about 40° C., from about 20° C. to about 40° C., from about 25° C. to about 40° C., from about 30° C. to about 40° C., or from about 35° C. to about 40° C.

Since the DNA splint molecule needs to hybridize with the entirety of the middle RNA segment, as well as several bases on both the left-arm RNA segment and the right-arm RNA segment, the length of the DNA splint molecule depends on the length of the middle RNA segment. In some embodiments, a length of the portion of the DNA splint molecule that hybridizes with the left-arm RNA segment and/or a length of the portion of the DNA splint molecule that hybridizes with the right-arm RNA segment ranges from about 4 bases to about 50 bases, such as from about 5 bases to about 40 bases, from about 6 bases to about 30 bases, or from about 7 bases to about 20 bases.

### RNA Product and Yield Thereof

In some embodiments, a length of the RNA molecule prepared by the method herein ranges from about 400 bases to about 6,000 bases, such as from about 500 bases to about 6,000 bases, or from about 750 bases to about 5,000 bases. In some embodiments, a length of the RNA molecule prepared by the method herein is about 400 bases, about 500 bases, about 600 bases, about 700 bases, about 800 bases, about 900 bases, about 1,000 bases, about 1,200 bases, about 1,500 bases, about 2,000 bases, about 2,500 bases, about 3,000 bases, about 3,500 bases, about 4,000 bases, about 5,000 bases, about 6,000 bases, or any ranges therebetween.

In some embodiments, a ligation yield of RNA molecule based on a molarity of the left-arm RNA segment, the middle RNA segment and/or the right-arm segment is about 20% or larger, such as about 25% or larger, about 30% or larger, about 35% or larger, about 40% or larger, or about 50% or larger.

In some embodiments, RNA molecule prepared by the method is substantially free of heterogeneity and mismatches around a ligation point between the left-arm RNA segment and the middle RNA segment, and a ligation point between the middle RNA segment and the right-arm RNA segment.

In some embodiments, the method further includes purifying the RNA molecule from the ligation mixture, and purifying the RNA molecule from the ligation mixture includes: subjecting the ligation mixture to an agarose gel electrophoresis; isolating an agarose gel section containing a band corresponding to the RNA molecule from the agarose gel; and isolating the RNA molecule from the isolated agarose gel section.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1

In some embodiments, the present study relates to an RNA sequencing strategy. According to the strategy, RNA molecules to be sequenced are first extended by the non-limiting example of RNA-dependent RNA polymerase, poliovirus 3D^{pol}, such that the RNA molecules are extended from the 3′ with the rest of the RNA molecule serving as templates.

The products of this replication/extension are double-stranded hairpin RNA molecules, which contain two-fold redundancy of most of the sequence information. These products can then be sequenced by, for example, nanopore sequencing technology. Since sequencing of the extended RNA includes sequencing both the native strand and the newly added complement strand, two layers of sequence information can be obtained at once, thus improving the accuracy of the sequencing.

Notably, the sequencing strategy was able to distinguish among nucleotides with modifications, including those of isomeric molecular mass, such as uridine (U) vs. pseudouridine (ψ), and m1A vs. m6A, by comparing the sequencing results of the native portion of the extended RNA with the sequencing results of the extended portion of the extended RNA. For example, it was discovered that nanopore sequencing method often mistakes pseudouridine for cytosine. When the nanopore sequencing results of a nucleotide indicates a mixture of cytosine and uridine in the native section and an adenosine at the corresponding location in the extended section, the novel sequencing strategy will be able to determine that the nucleotide at that location was a pseudouridine.

### Example 2: Detection of Pseudouridine Modifications and Type I/II Hypermodifications in Human mRNAs Using Direct, Long-Read Sequencing

Enzymatic modifications to mRNAs have the potential to fine-tune gene expression in response to environmental stimuli. Notably, pseudouridine-modified mRNAs are more resistant to Rnase-mediated degradation, more responsive to cellular stress, and have the potential to modulate immunogenicity and enhance translation in vivo. However, the precise biological functions of pseudouridine modifications remain unclear due to the lack of sensitive and accurate mapping tools. The present study developed a semi-quantitative method for high-confidence mapping of pseudouridylated sites on mammalian mRNAs via direct long-read nanopore sequencing. A comparative analysis of a modification-free transcriptome reveals that the depth of coverage and intrinsic errors associated with specific k-mer sequences are critical parameters for accurate basecalling. These parameters were adjusted for high-confidence U-to-C basecalling errors that occur at pseudouridylated sites, and benchmark against sites that were previously identified in human rRNA or mRNA using biochemical methods. Using the method, new pseudouridylated sites were uncovered, many of which fall in k-mers that are known targets of pseudouridine synthases. Sites identified by U-to-C base calling errors were quantified using 1000-mer synthetic RNA controls bearing a single pseudouridine in the center position. The quantification approach demonstrates that while U-to-C basecalling error occurs at the site of pseudouridylation, this basecalling error is systematically under-called at the pseudouridylated sites. The sequencing method was used to discover mRNAs with up to 7 unique sites of pseudouridine modification. The pipeline allows direct detection of low- and high-occupancy pseudouridine modifications on native RNA molecules from nanopore sequencing data without resorting to RNA amplification, chemical reactions on RNA, enzyme-based replication, or DNA sequencing steps.

### Example 2

Enzyme-mediated RNA chemical modifications have been extensively studied on highly abundant RNAs such as transfer RNAs and ribosomal RNAs, however, it is now known that messenger RNAs are also targets of RNA modification. Although modifications occur to a lesser extent in mRNAs than other RNAs, these modifications potentially impact gene expression, RNA tertiary structure formation, or the recruitment of RNA-binding proteins. Pseudouridine (psi) is synthesized from uridine converted in vivo by one of more than a dozen pseudouridine synthases identified to date. It was the first discovered RNA modification and represents 0.20-6% of total uridines in mammalian mRNAs. Psi-modified mRNAs are more resistant to Rnase-mediated degradation and also have the potential to modulate splicing and immunogenicity and alter translation//www.biorxiv.org/content/10.1101/2021.11.03.467190v2.full#ref-12 in vivo. Further, psi modifications of RNAs are responsive to cellular stress, leading to increased RNA half-life. Little is known about the biological consequences of pseudouridylation, except for a few well-studied cases. For example, defective pseudouridylation in cells leads to disease, including X-linked dyskeratosis congenita, a degeneration of multiple tissues that severely affects the physiological maintenance of ‘stemness’ and results in bone marrow failure. A critical barrier to understanding the precise biological functions of pseudouridylation is the absence of high-confidence methods to map psi-sites in mRNAs. Psi modifications do not affect Watson-Crick base pairing, thereby making them indistinguishable from uridine in hybridization-based methods. Additionally, the modification bears the same molecular weight as the canonical uridine, making it challenging to detect directly by mass spectrometry.

Psi is conventionally labeled using N-cyclohexyl-N′-b-(4-methylmorpholinium) ethylcarbodiimide (CMC), a reagent that modifies the N1 and N3 positions of psi, N1 of guanine, and the N3 of uridine. Treatment with a strong base removes the CMC from all the sites except for the N3 position of psi. Recently, the use of an RNA bisulfite reaction was demonstrated for the specific labeling of psi. Indirect chemical labeling of psi combined with next-generation sequencing has yielded over 2,000 putative psi sites within mammalian mRNAs, but different methods identified different sites and the overlap is limited, pointing to a need for improved detection and quantification technology. Reliance on an intermediate chemical reaction (i.e., CMC or RNA bisulfite) can lead to false-positive or false-negative results due to incomplete labeling or stringent removal of reagent from the N1 position of psi. Further, each of these methods relies on the amplification of a cDNA library generated from the chemically modified mRNAs, leading to potential false positives from biased amplification. Finally, since these methods rely on short reads, it is difficult to perform combinatorial analysis of multiple modifications on one transcript.

Nanopore-based direct RNA sequencing has been used to directly read RNA modifications. In these reports, ion current differences for different k-mer sequences (k=5 nucleotides) as an RNA strand is moved through the pore suggest the presence of a modified RNA base. Detection of psi using nanopores was also confirmed for rRNAs, for the Saccharomyces cerevisiae transcriptome, and for viral RNAs, as indicated by a U-to-C base-calling error at various sequence sites. Algorithms for psi quantification have been produced for various k-mers using combinatorial sequences that contain psi sites within close proximity as well as control RNAs containing many natural RNA modifications, also in close proximity (e.g., rRNA). While these control molecules allow many k-mers to be studied, the accuracy of quantifying psi occupancy at a given modified site can be highly dependent on the nucleotide sequence surrounding the modification. Moreover, sequence context is particularly important for the measurement of RNA molecules wherein the secondary structure can influence the kinetics of translocation as mediated by the helicase. Control molecules for psi modification that match the transcriptome sequence beyond the context of the measured k-mer are more desirable than random sequences.

According to some embodiments, a nanopore-based method to accurately map psi modifications in a HeLa transcriptome by comparing the sequence alignment to identical in vitro IVT controls without RNA modifications was tested. It was demonstrated that the number of reads and specific k-mer sequences are critical parameters for defining psi sites and for assigning significance values based on these parameters, enabling making high-confidence and conservative, binary identifications of psi modification sites, transcriptome-wide. The approach recapitulates 198 previously annotated psi sites, 34 of which are detected by 3 independent methods, thus providing a “ground truth” list of psi modifications in HeLa cells. The approach also reveals 1,691 putative psi sites that have not been reported previously. It is shown that these new sites tend to occur within k-mer sequences including the PUS7 and TRUB1 sequence motifs that were previously reported.

The accuracy of the algorithm for detecting sites of psi modifications was validated using ribosomal RNAs which have been comprehensively annotated by mass spectrometry and assigned 41/46 psi modification using the present method. Additionally, five 1,000-mer synthetic RNA controls containing either uridine or psi at a known pseudouridylated position in the human transcriptome were synthesized and analyzed. This quantitative analysis revealed that U-to-C mismatch errors are systematically under-called for the detection of psi, thus enabling us to apply a basecalling error cutoff to identify 40 high-occupancy, hypermodified type I psi sites, which are likely to confer measurable phenotypes. The present study discovered that these sites tend to occur in k-mer sequences for which uridine and guanine precede the pseudouridylated site. In accordance with previous findings that show higher median psi-ratio for positions with the TRUB1 and the PUS7 sequence motifs as compared to the other k-mers.

Further, with methods developed in the present study, 38 mRNAs were identified with multiple high-confidence psi sites, which are confirmed by single-read analysis. Interestingly, mRNAs with up to 7 unique psi sites were found. Combined, this work reports a pipeline that enables direct identification and quantification of the psi modification on native mRNA molecules, without requiring chemical reactions on RNA or enzyme-based amplification steps. The long nanopore reads allow, in principle, the detection of multiple modifications on one transcript, which can shed light on cooperative effects on mRNA modifications as a mechanism to modulate gene expression.

### Example 3: Synthesis of Long RNA with a Site-Specific Modification by Enzymatic Splint Ligation

Synthesis of RNA molecules that contain an internal site-specific modification is important for RNA research and therapeutics. While solid-state synthesis is attainable for such RNA in the range of 100 nucleotides (nts), it is currently impossible with kilobase (kb)-long RNA. Instead, long RNA with an internal modification is usually assembled in an enzymatic 3-part splint ligation to join a short RNA oligonucleotide, containing the site-specific modification, with both a left-arm and a right-arm long RNA that are synthesized by in vitro transcription. However, long RNAs have structural heterogeneity and those synthesized by in vitro transcription have 3′-end sequence heterogeneity, which together substantially reduce the yield of 3-part splint ligation. Here the present study developed a method of 3-part splint ligation with an enhanced efficiency utilizing a ribozyme cleavage reaction to address the 3′-end sequence heterogeneity and involving DNA disruptors proximal to the ligation sites to address the structural heterogeneity. The yields of the synthesized kb-long RNA are sufficiently high to afford purification to homogeneity for practical RNA research. The present study also verified the sequence accuracy at each ligation junction by nanopore sequencing.

### Example 3-1

Synthesis of kb-long RNA with an internal modification is important for probing the structure and function of the epitranscriptome. Matured mammalian mRNAs are now known to contain post-transcriptional modifications. While each modification in mRNA occurs at a much lower frequency relative to tRNA and rRNA, each may confer information that regulates gene expression at a complexity that opens a new avenue of research. A critical barrier to progress is the lack of a robust method that precisely maps the position of each mRNA modification in the epitranscriptome. For example, the modification pseudouridine (ψ) in mRNA does not affect Watson-Crick base pairing and thus cannot be detected by hybridization-based sequencing methods, such as by reverse-transcription of RNA during cDNA synthesis for Illumina sequencing. Importantly, nanopore sequencing of RNA has emerged as an attractive alternative. In this technology, native mRNA is fed directly into the flow cell without the need to convert into cDNA. Detection of ion current differences for different k-mer sequences (k=5) during translocation of the RNA through the pore suggests the presence of a modified nucleotide. However, while the nanopore platform has the unique advantage of producing direct and long reads in a high-throughput capacity, each modification is read as basecalling “mismatch” errors. The significance value of each mismatch error is dependent on the sequence context, due to the presence of RNA intramolecular structures that can influence the kinetics of RNA translocation through the pore. To quantify the potential of using the basecalling error as an indicator for a modification, the technology needs a synthetic mRNA control with the modification at its homogeneity (i.e., 100%) and in its natural sequence context. This synthetic control is a necessary reference to determine the level of detection by the basecalling error in nanopore sequencing. Additionally, besides contributing to nanopore RNA sequencing technology, long RNA with an internal modification is of great interest to current efforts in RNA therapeutics and vaccine development.

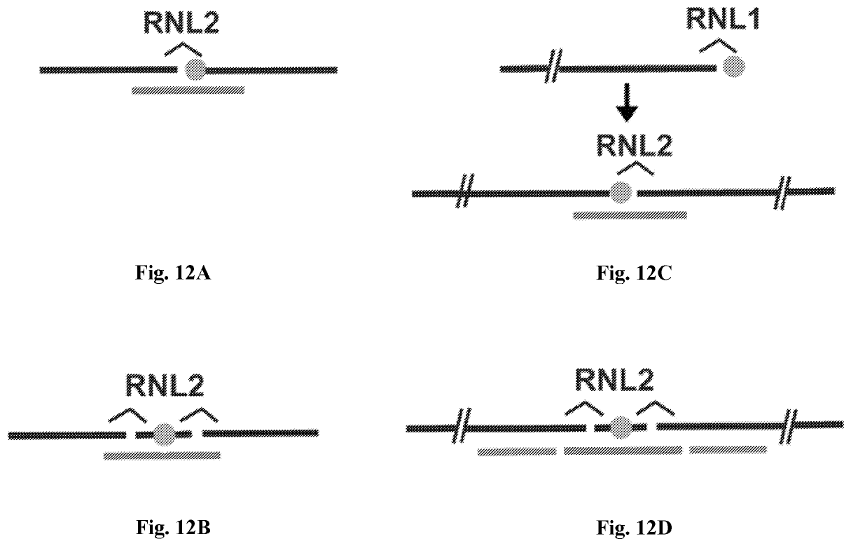

Synthesis of short RNA (<100 nts) with an internal modification is achievable through the solid-phase platform of chemical coupling. This platform, however, is expensive and has a steep decline in product yield with increasing RNA length. In a more cost-effective approach, short RNA with a modification can be synthesized by a 2-part splint ligation that joins two RNAs, one of which contains the modification, on a complementary single-stranded DNA splint (FIG. 12A). If the two arms share complementary sequences, such as those that constitute a full-length tRNA, the enzymatic joining can be facile even without a splint. The joining of two single-stranded RNAs in the absence of a splint is preferred by T4 RNA ligase 1 (RNL1), whereas the joining of a nicked RNA in the presence of a splint is preferred by T4 RNA ligase 2 (RNL2). The joining can also be achieved by 3-part ligation (FIG. 12B), where the middle RNA is synthesized by a solid-phase approach with the site-specific modification, whereas the two side RNAs are each made by in vitro transcription, usually with T7 RNA polymerase (RNAP). The three RNAs are aligned on a DNA splint and are joined by RNL2. For assembly and synthesis of a short RNA, the efficiency of 2-part or 3-part ligation is typically 30-60%, which is a practical yield that affords purification and subsequent analysis. Even if the short RNA has a stable structure, such as the well-defined structure of a tRNA, the ligation position can be chosen to minimize structural interference.

In contrast, kb-long RNA cannot be generated in full-length by solid-phase synthesis. The current technology of solid-phase synthesis is limited to fewer than 200 nts but with poor yield and frequent synthesis failure. Instead, kb-long RNA must be assembled from fragments by a combination of enzymatic and chemical synthesis. One such method employs RNL1-dependent extension of an in vitro transcribed left-arm RNA with a modified nucleotide, which is then joined by an RNL2-mediated splint ligation with the right-arm RNA, also generated by in vitro transcription (FIG. 12C). In this method, the modified nucleotide is synthesized with 3′,5′-bisphosphates, which restrict extension of the left-arm to a single nucleotide using the 3′-phosphate as a block. After dephosphorylation of the 3′-phosphate, the extended left-arm is joined with the right-arm by a 2-part splint ligation. While this method successfully generated a ˜400-mer RNA, synthesis of the modified nucleotide with 3′,5′-bisphosphates requires special expertise. A more general method is a 3-part splint ligation to join a chemically synthesized short RNA that bears the site-specific modification with a left-arm and a right-arm RNA, the latter of which are synthesized by in vitro transcription. However, the yields have been low (<2%), preventing practical purification and subsequent study of the ligated RNA.

Here, the present study developed a method that improves the yield of 3-part ligation to synthesize kb-long RNA containing a site-specific internal modification. The innovation is the combination of two strategies that address key issues of low yield. First, in vitro transcribed RNA usually has a population of 3′-ends, due to the propensity of RNAPs to prematurely terminate, and alternatively, to extend beyond the 3′-end with extra non-templated nucleotides. This problem was previously addressed on short RNA by transcribing the RNA with a cis-acting ribozyme that would self-cleave, leaving the dissociated short RNA with a homogeneous 3′-end. Second, long RNA has inherent structural heterogeneity, which lacks a well-defined tertiary structure but folds and re-folds spontaneously and dynamically with the ability to engage both termini in intramolecular base pairing, thus blocking them from splint ligation. It was found that using DNA disruptors to hybridize to RNA sequences near each ligation site or using an ultra-long DNA splint (up to 100 nts) improves the ligation yield, presumably by freeing up the RNA termini. Both strategies were incorporated into a single method that improves the ligation yield by 3-5-fold over the best reported yields. In this method, a cis-acting ribozyme was engineered to the left-arm RNA to produce a precise 3′-end, and two DNA disruptors were included to hybridize next to the ligation sites in the ligation reaction. In addition, the present study proposes for the first time a method to purify 1 kb-long RNA for sequence verification of ligation accuracy, using nanopore sequencing at single-molecule resolution. Combined, this method demonstrates the ability to generate kb-long RNA bearing a site-specific modification for broader research.

### Example 3-2: Materials and Methods

### Synthesis of Left-Arm and Right-Arm RNAs by In Vitro Transcription

The template for in vitro transcription for synthesis of a left-arm or a right-arm RNA, each ˜500-mer and unmodified, is made by solid-phase synthesis as a double-stranded gBlock DNA by IDT (Integrated DNA Technologies). A gBlock double-stranded DNA is designed with the consensus T7 promoter sequence, followed by the sequence of interest beginning with three G nucleotides to facilitate transcription. The gBlock for the left-arm RNA additionally encodes the sequence for the Hepatitis delta virus (HDV) ribozyme (Chowrira et al., J Biol Chem, 269, 25856-25864 and Been et al., Biochemistry, 31, 11843-11852), which upon synthesis by transcription can self-cleave to release the left-arm RNA. The HDV ribozyme has the sequence:

(SEQ ID NO: 1)
5′-GGGUCGGCAUGGCAUCUCCACCUCCUCGCGGUCCGACCUGGGCUAC
UUCGGUA-GGCUAAGGGAGAAG-3′

Left-arm and right-arm gBlocks (500 ng, ˜1.5 pmoles) were transcribed at 37° C., 2h, in 20 μL using the NEB HiScribe kit. Because the right-arm RNA must have a 5′-monophosphate (5′-pG) to participate in the ligation reaction, its transcription reaction was supplemented with 20 mM GMP and 10 mM MgCl₂. After transcription, the gBlock DNAs were hydrolyzed by Rnase-free Dnase I (NEB) at 37° C., 15 min, and the RNA products were isolated using the NEB 50 μg-scale Monarch RNA Cleanup cartridges. The yield of purified RNA is usually >100 μg.

Each in vitro transcribed RNA was determined for the concentration by A₂₆₀ (usually in a 1:50 dilution of the stock) and analyzed (usually 1 μL of the 1:50 dilution) in a 6% denaturing PAGE/7M urea gel (abbreviated as denaturing PAGE hereafter). The gel was run in 1X TBE (90 mM Tris, pH 8.0, 90 mM boric acid, 2 mM Na₂EDTA) in a Bio-Rad mini-Protean apparatus for 30-60 min at 200 V at 60° C., along with a low MW (molecular weight)-range RNA ladder (NEB). SYBR Gold-stained gels were imaged to determine the fraction of intact RNA in each sample, which was used to adjust the RNA concentration as determined by A₂₆₀ to more accurately reflect the concentration that would participate in the ligation reaction. Additional assessment of the RNA concentration was obtained by comparing the RNA band intensity to the known amount of a standard RNA in the same gel.

### Ribozyme Self-Cleavage of the Left-Arm RNA to Generate a Homogeneous 3′-End

While HDV can catalyze self-cleavage during transcription, this activity is usually at a low level. To produce a higher level of cleavage for better ligation yield, several heat-cool cycles were performed. The in vitro transcribed left-arm RNA (200 pmoles) was mixed with a 60-mer left-arm DNA disrupter (2 nmoles) in 90 μL of 110 mM Tris-Oac (Tris acetate, pH 6.3). The reaction was incubated at 85° C., 2 min, cooled to room temperature, and supplemented with 5 μL 200 mM MgCl₂ and 5 μL 200 mM 2-mercaptoethanol ((3-Me) to a final volume of 100 μL (at the final concentration of 10 mM MgCl₂, 10 mM 3-Me, 20 μM of the left-arm DNA disruptor, and 2 μM of the left-arm RNA). The reaction was transferred to a PCR tube and incubated in a thermocycler at 72° C., 30 s, followed by 4 heat-cool cycles each lasting 15 min between 72° C. and 8° C. The yield of the cleavage was determined by the fraction of the cleaved product in the total input left-arm RNA. The A₂₆₀ was not informative, due to the presence of disruptor DNA.

### Hydrolysis of 2′,3′-Cyclic Phosphate on the Left-Arm RNA Generated by HDV Cleavage

HDV cleavage of the transcribed left-arm RNA produces a 2′,3′-cyclic phosphate at the 3′-end, which was hydrolyzed by adding 1.5 μL of 10 units/μL T4 PNK (polynucleotide kinase) and 1 μL Rnase-Out solution (40 units/μL, ThermoFisher) to the cleavage reaction above. The hydrolysis reaction was incubated at 37° C., 8 h, while aliquots of 0.4 μL were analyzed on a 6% analytical denaturing PAGE. Each reaction aliquot as well as a final reaction mixture was extracted with phenol:chloroform:isoamyl alcohol (25:24:1), pH 5, and the RNA was ethanol precipitated with 1/10 vol of 2.5 M NaOAc, pH 5.0, and 3 vol ethanol. The pellet was washed, air dried, and dissolved in 20 μL Rnase-free water. Hydrolysis was verified by ligation of the T4 PNK-treated left-arm RNA with a 15-mer RNA in a 2-part ligation reaction, using the same splint and conditions as in the 3-part ligation reaction.

### Design of DNA Disruptors for 3-Part Splint Ligation

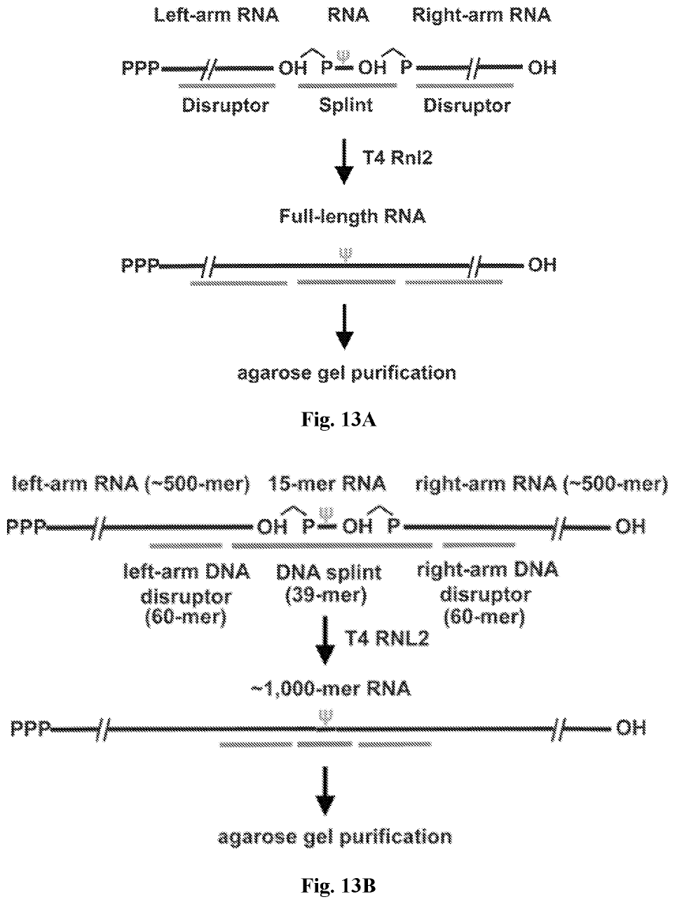

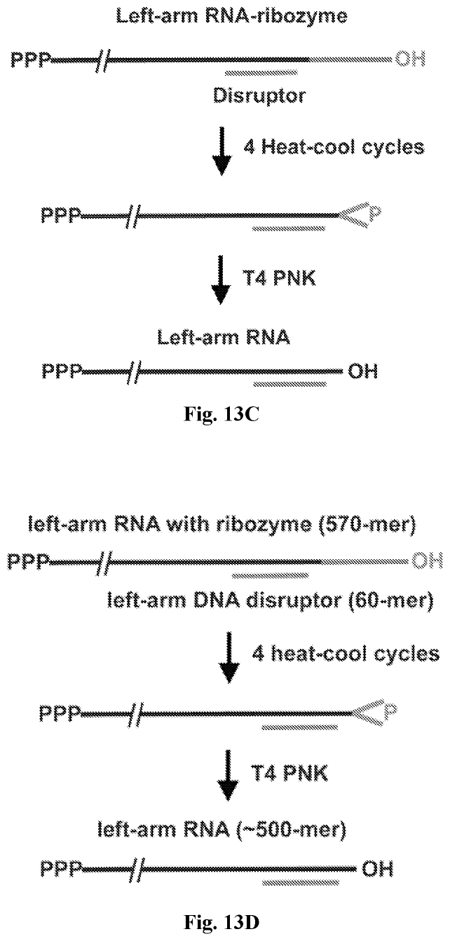

DNA 60-mer disruptors were designed to hybridize to the left-arm and right-arm RNA adjacent to the 3′- and 5′-end of the DNA splint in each 3-part splint ligation reaction (see FIG. 13A). These DNA disruptors were synthesized by IDT without purification.

### Three-Part Splint Ligation

The molar ratios below provide a framework to optimize each reaction. A typical 3-part ligation reaction consists of 1:1:1.5 ratio of the left-arm RNA, the right-arm RNA, and the 15-mer RNA that is chemically synthesized with a site-specific modification. These RNAs are then mixed with a 24:24:0.9 molar ratio of the left-arm DNA disruptor (60-mer), the right-arm DNA disruptor (60-mer), and the DNA splint (39-mer). For the reactions described here, the molar ratios represent 15 pmoles of the ribozyme-cleaved and T4 PNK-treated left-arm RNA, 15 pmoles of the right-arm RNA, 22.5 pmoles of the 15-mer RNA with a modification, 360 pmoles of the left-arm DNA disruptor, 360 pmoles of the right-arm DNA disruptor, and 13.5 pmoles of the splint DNA.

All components of a 3-part splint ligation reaction were mixed in a PCR tube according to the molar ratios above with 3.6 μL of 0.5 M HEPES, pH 7.5, in a total volume of 27 μL. The 3-part ligation reaction was initiated at 40° C., 5 min, and cycled down to 5° C. by decreasing 5° C. every 2 min. The annealed 3-part pre-ligation complex was mixed with 9 μL of a 4× ligation sub-stock to 36 μL and incubated at 16° C., 15 min, 25° C., 30 min, and 37° C., 60 min, in a thermocycler. The 4× ligation sub-stock contains 8 mM MgCl₂, 2 mM ATP/Mg²⁺, 4 mM DTT, 14 μM RNL2, and 2 units/μL Rnase-Out. Each 3-part splint ligation was performed in 36 μL with the final concentration of 0.42 μM 3′-end processed left-arm RNA, 0.42 μM right-arm RNA, 0.62 μM 15-mer RNA with a site-specific modification, 10 μM each of the left-arm and right-arm disruptors, 0.38 μM splint DNA, 50 mM HEPES, pH 7.5, 2 mM MgCl₂, 0.5 mM ATP/Mg²⁺, 1.0 mM DTT, 3.5 μM RNL2, and 0.5 units/μL Rnase-Out.

### Clean-Up of the 3-Part Splint Ligation Reaction

Once completed, the 3-part ligation reaction was diluted to 150 μL with Rnase-free water, supplemented with 15 μL 2.5 M NaOAc, pH 5.0, 1 μL 20 μg/μL glycogen, and extracted twice with equal volumes of phenol:chloroform:isoamyl alcohol (25:24:1), pH 5.2. Following an ethanol precipitation, the nucleic acid pellet was dissolved in 15 μL 70% deionized formamide by heating at 65° C., 5 min. To determine the efficiency of 3-part vs. 2-part ligation, an aliquot of 0.6 μL was run on a 6% denaturing PAGE. Typical yields are 10-35% for 3-part ligation and 35-65% for 2-part ligation.

### Gel Purification of the Ligated 1 kb RNA for Nanopore Sequencing

Ligation workups from the previous step were supplemented with 3 μL of 6× purple gel loading dye (NEB), heat denatured at 85° C. for 1 min, and electrophoresed at 100 V, 1 h, in an 1.2% agarose gel (8×7 cm) with 6 wells in TAE buffer (40 mM Tris-acetate, pH 8.3, 1 mM EDTA) (Masek et al., Anal Biochem, 336, 46-50). An authentic 1 kb RNA standard was included as a reference. The ethidium bromide-stained gel was visualized on a BioRad ChemiDoc imaging system, and a paper printout of the image was used as a guide to excise the 1 kb bands of interest. From the agarose gel, 40-50% of the input 1 kb RNA (usually 160-250 ng) was recovered intact in 15 μL water using the Zymo Clean RNA gel recovery kit. Concentration of the RNA was determined using the Qubit RNA HS assay kit and its integrity was assessed by the Agilent 2100 Bioanalyzer with an RNA Nanoreagent chip. Gel-purified 1 kb RNAs from 3-part ligation were combined for Direct RNA sequencing (SQK-RNA002) with the ONT direct RNA sequencing protocol (DRCE_9080_v2_revH_14Aug2019) as described in Tavakoli et al. (Nat Commun.). Basecalling, alignment, and signal intensity extraction was as described in Tavakoli et al., as well.

### Affinity Purification of the Ligated 1 Kb RNA

Each 3-part splint ligation reaction (36 μL) was extracted twice with phenol:chloroform:isoamyl alcohol (25:24:1), pH 5.0, followed by ethanol precipitation or cleanup through a Zymo RNA Clean and Concentrator-5 cartridge. The recovered RNA, consisting of the 1 kb full-length, the left-arm and right-arm 500-mers, and the 15-mer, was supplemented with 75 pmoles of the biotinylated 39-mer splint and 750 pmoles each of the left-arm and right-arm disruptors in 80 μL of gel elution buffer (0.1% SDS, 1 mM EDTA, 0.3 mM NaOAc, pH 5.0). This solution was carried through a slow heat-cool from 65° C. to 5° C. It was then added to 40 μL of washed streptavidin-sepharose resin (Cytiva) in gel elution buffer and the mixture was incubated 30-60 min with 1,500 rpm rotation at room temperature. After a brief centrifuge of the suspension at 7,500 rpm, the supernatant was removed. The remaining resin was washed 3 times with 200 μL each of the gel elution buffer and once with F (10 mM Tris-HCl, pH 8.0). The washed resin was resuspended in 200 μL of fresh TE, and the bound RNA was released to the supernatant by heating at 65° C., 5 min. After a brief spin, the RNA in the supernatant was precipitated with isopropanol in the presence of 20 μg glycogen, washed, and air dried.

MRPS14
15-mer RNA: p-CUUGGACΨUAACAAA (SEQ ID NO: 2)
Left-arm gBlock:
TAATACGACTCACTATAGGGTCACTATGTAGACTGGAGAATGTGGCGCGATGTGA
AGAGACGAAAAATGGCCTATGAATACGCAGATGAGAGGCTACGTATTAATTCACT
CAGGAAGAATACCATTTTGCCAAAAATTCTTCAGGATGTGGCTGATGAAGAAATT
GCTGCCCTCCCCCGGGATAGCTGTCCTGTTAGAATCAGAAATCGGTGTGTTATGAC
GTCCCGTCCGCGTGGTGTGAAGCGGCGCTGGAGGCTTAGTCGTATAGTCTTCCGTC
ACTTAGCTGACCATGGGCAACTTTCTGGGATCCAGCGAGCGACATGGTAAATGAG
CTCCAGAACCTATTGAGCTTGCAGGGAAGCCAAGCTTGCAGTTCCAGCAAGCAAA
GATTTTTTTTAATAGACCAAACCCTAATCTCTACAGGGGCCCAGTACAGTTGTTTG
GCCTACCTGATGCTATCTCTAAACTACTTTTAAAATGAAGACATTTGGGTGTTTCA
TGTCAGTGAATTATCTTTTCTGGGTCGGCATGGCATCTCCACCTCCTCGCGGTCCG
ACCTGGGCTACTTCGGTAGGCTAAGGGAGAAG (SEQ ID NO: 3)
Right-Arm_gBlock:
TAATACGACTCACTATAGGGCATACTGTTCCCATACATAGTACACCATGTTGTATA
ATAAATATTTTCTGTGAATGTTTTTTATACCCTCCTTTTAAGTTTAATCTTTTAGTA
ACATATAGGGGTTTTTGTATTAAGAGCATTATTTCATAAAATGCTATCTTATACAT
AATCCAACTAATTGACATGTAGCTCATGTATGGCAGTTTGTAATCCTGGACTAGCT
TTGCTGACCTGCAGAGCTCCCCTTAACTATTATTGTTAGATAAACAGATTGGATAG
CAAGGCTGCTGTTCTGGATTATCTAATGCTCCACTTCTTCTTGTGATGGCTGAACA
AAAGAACCACTAAGTGTTTTCTTGTCCATCCTTAAGGGACTTTAAAACAAATTATG
CCTCTTTAAGTCCTTGAAAGTCTCCATCTAAAGAGGAAAAGTTTGTTGGAACAGTA
TATATTCCCTATTTTTATGTTTATTTAATCCATAACAGAAATGAGTAAGGGTAGCA
CAAAGTTCTCATTCA (SEQ ID NO: 4)
DNA splint:
AACAGTATGCCCTTTGTTAAGTCCAAGAGAAAAGATAAT (SEQ ID NO: 5)
Left-arm disruptor:
CACTGACATGAAACACCCAAATGTCTTCATTTTAAAAGTAGTTTAGAGATAGCATC
AGGT (SEQ ID NO: 6)
Right-arm disruptor:
GAGGGTATAAAAAACATTCACAGAAAATATTTATTATACAACATGGTGTACTATG
TATGC (SEQ ID NO: 7)
PRPSAP1
15-mer RNA: p-GCCCGGAΨUGAUGGG (SEQ ID NO: 8)
Left-arm gBlock:
TAATACGACTCACTATAGGGTGATGATGGCCAAAGAGAAGCCACCGATAACTGTA
GTTGGAGATGTTGGAGGCCGCATCGCAATCATCGTGGATGACATTATTGACGATG
TGGAGAGTTTTGTTGCTGCCGCGGAGATCCTGAAAGAGAGAGGCGCCTATAAGAT
CTATGTTATGGCCACCCACGGCATCCTGTCTGCAGAGGCCCCTCGCCTGATTGAGG
AGTCCTCCGTAGACGAGGTGGTGGTGACGAATACTGTCCCTCATGAGGTTCAGAA
GCTGCAATGTCCCAAGATAAAGACTGTGGATATCAGTTTGATTCTTTCTGAAGCCA
TTCGGAGAATCCACAATGGAGAGTCCATGGCCTACCTTTTCCGAAACATCACTGTG
GATGACTAGCTTTCACGAGGGTCTCGACCCTGGACCTCCTGAGGGAAACATGGAA
AAAGCAGTGCCATGAGTGATACAGTGTTTCCTTGCAAGGGAGGACTCGAAACAGC
CTGGAGTTAGATATCTTCTTTTGGGTCGGCATGGCATCTCCACCTCCTCGCGGTCC
GACCTGGGCTACTTCGGTAGGCTAAGGGAGAAG (SEQ ID NO: 9)
Right-arm gBlock:
TAATACGACTCACTATAGGGGAGGAGGGATTAAAAGAGTCAGGACCATGTTGTAG
CTAATGGATAAATATCATAACATGGCCTTACATGTCTGCTGTCATCAGCCCTGTTC
CTTAAAAGTTCTAGCTGCTTTCTTAAAAATAATCTGAAAATCTTATTGATACTAAA
GAGGAGTTAAAGGCACATAAAGTCTTAACTCTATAATGTTCATTTAGTTGTTTCAG
CTCCAGGGAAATGGAGGTATTGATGTTGAACCTGGTTAGGGAAGCTGAGCGCCTG
TGGCCCTATTACTATCCAGTTGGCCTCTCCCAAATCAACTTCAAGTCTTTTATAGA
GAATCGTATTTTTCTTTCAGAAATTGTTATGCCTACAGCCATTGAAAAATGAAGCA
TTCATGTTGTTACATCTTCCAAGGATGTCAGATTAGAAAATAGCATCCCACCTCTG
GGTATCTGAGTGGCTCTGAAGTTGCAAATAAAATAATTTGTTGTCTTGCTAGTCTT
TTCATTAGACAGCAGAAG (SEQ ID NO: 10)
DNA splint:
TCCCTCCTCCCCCCCATCAATCCGGGCAAAAGAAGATAT (SEQ ID NO: 11)
Left-arm disruptor:
TAACTCCAGGCTGTTTCGAGTCCTCCCTTGCAAGGAAACACTGTATCACTCATGGC
ACTG (SEQ ID NO: 12)
Right-arm disruptor:
ACATGTAAGGCCATGTTATGATATTTATCCATTAGCTACAACATGGTCCTGACTCT
TTTA (SEQ ID NO: 13)
PSMB2
15-mer RNA: p-UCAGUGUΨCGAAUCA (SEQ ID NO: 14)
Left-arm gBlock:
TAATACGACTCACTATAGGGCTATGTTCTTGTCGCCTCCGACCGGGTGGCCGCCAG
CAATATTGTCCAGATGAAGGACGATCATGACAAGATGTTTAAGATGAGTGAAAAG
ATATTACTCCTGTGTGTTGGAGAGGCTGGAGACACTGTACAGTTTGCAGAATATAT
TCAGAAAAACGTGCAACTTTATAAGATGCGAAATGGATATGAATTGTCTCCCACG
GCAGCAGCTAACTTCACACGCCGAAACCTGGCTGACTGTCTTCGGAGTCGGACCC
CATATCATGTGAACCTCCTCCTGGCTGGCTATGATGAGCATGAAGGGCCAGCGCT
GTATTACATGGACTACCTGGCAGCCTTGGCCAAGGCCCCTTTTGCAGCCCACGGCT
ATGGTGCCTTCCTGACTCTCAGTATCCTCGACCGATACTACACACCGACTATCTCA
CGTGAGAGGGCAGTGGAACTCCTTAGGAAATGTCTGGAGGAGCTCCAGAAACGCT
TCATCCTGAATCTGCCAACCTGGGTCGGCATGGCATCTCCACCTCCTCGCGGTCCG
ACCTGGGCTACTTCGGTAGGCTAAGGGAGAAG (SEQ ID NO: 15)
Right-arm gBlock:
TAATACGACTCACTATAGGGTTGACAAAAATGGCATCCATGACCCCATGTTGTATT
CCTTCCCCAAACAGGGCTCCTAACATCATGTCCTCCCTCCCACTTGCCAGGGAACT
TTTTTTTGATGGGCTCCTTTATTTTTTTCTACTCTTTTCAGGCGCACTCTTGATAAAT
GGTTAATTCAGAATAAAGGTGACTATGGATATAATTGAGCCCTCTGGTCCAGGTCT
CAGTTTACCTAATATTACCTCAGAAAGGATATGGAGGGAAGATGATCTTTTTGCCA
GGTCTGACTTTTCTTCCTGCTCCGCCCTCCATTAACGCTCAGTACCCTTTAGCAGCT
GACGGCCCCACGTTCTACTCCATGCTTGGCTTCCTTTCCAACTAGCTCTTTCATATA
TTTTACTTGCTAGTATCTCCATTCTCTCTAAAGTAGTGGTTCTTTTTGCCCTTAAAC
TTAAATTTTTAAATTAATTAACCTGAATTAATAATACATGCACTTAATGTAACATG
CAAACAGTACA (SEQ ID NO: 16)
DNA splint:
TTTTGTCAACCCTGATTCGAACACTGAAGGTTGGCAGAT (SEQ ID NO: 17)
Left-arm disruptor:
CAGGATGAAGCGTTTCTGGAGCTCCTCCAGACATTTCCTAAGGAGTTCCACTGCCC
TCTC (SEQ ID NO: 18)
Right-arm disruptor:
AGGACATGATGTTAGGAGCCCTGTTTGGGGAAGGAATACAACATGGGGTCATGGA
TGCCA (SEQ ID NO: 19)
MCM5
15-mer RNA: p-GGAGCUGΨAGUGUCC (SEQ ID NO: 20)
Left-arm gBlock:
TAATACGACTCACTATAGGGTGCGCATCGCGGAAGCCCTCAGCAAGATGAAGCTG
CAGCCCTTCGCCACAGAGGCAGATGTGGAGGAGGCCCTGCGGCTCTTCCAAGTGT
CCACGTTGGATGCTGCCTTGTCCGGTACCCTGTCAGGGGTGGAGGGCTTCACCAGC
CAGGAGGACCAGGAGATGCTGAGCCGCATCGAGAAGCAGCTCAAGCGCCGCTTT
GCCATTGGCTCCCAGGTGTCTGAGCACAGCATCATCAAGGACTTCACCAAGCAGA
AATACCCGGAGCACGCCATCCACAAGGTGCTGCAGCTCATGCTGCGGCGCGGCGA
GATCCAGCATCGCATGCAGCGCAAGGTTCTCTACCGCCTCAAGTGAGTCGCGCCG
CCTCACTGGACTCATGGACTCGCCCACGCCTCGCCCCTCCTGCCGCTGCCTGCCAT
TGACAATGTTGCTGGGACCTCTGCCTCCCCACTGCAGCCCTCGAACTTCCCAGGCA
CCCTCCTTTCTGCCCCAGAGGAAGGGTCGGCATGGCATCTCCACCTCCTCGCGGTC
CGACCTGGGCTACTTCGGTAGGCTAAGGGAGAAG (SEQ ID NO: 21)
Right-arm gBlock:
TAATACGACTCACTATAGGGTGCTGCCTCTGGGCGCCCGCCTCTCCATGTTGTATG
GGAAGTGTGCTTTTGGCATCCGTTAATAATAAAGCCACGGTGTGTTCAGGTATCTG
TGGGTTTGTGCACTCGGTGACCTGTCACCTCCATCGTGCCCTCATGGCAGGGTAAG
TGTGAGGGAACAGGGTCTGGAGGCAGACTGGCCAGGGTTTCTGACTTGGATCTGC
CACTCAGACTTCTGGGTAAGTCACGTGACTTGAGTGCTCCGGTTTCTTCATCTGTTT
AGCGGGGCTCATAAAGCCACTCCCTTGATGCTGGGAATCCAGTGAGACCAGGGAG
TGGGGAAGCGCTTCTCAAAGTGTGTGATTTTGATCCGCCTCACCTGTTCCCCAGTA
ACTTATTGGGGGGCTGCGAGCCGTGGTGCGGCGGTCAGACTGCCTGGATTCATGG
ACCCTCTCCATGCCTTAGTTTCCTTATCTGTAAGCAGGCTGATACTAGAACCTGCT
TCACAGGGTTGTGCTTAG (SEQ ID NO: 22)
DNA splint:
GAGGCAGCACCCGGACACTACAGCTCCTTCCTCTGGGGC (SEQ ID NO: 23)
Left-arm disruptor:
GAAAGGAGGGTGCCTGGGAAGTTCGAGGGCTGCAGTGGGGAGGCAGAGGTCCCA
GCAACA (SEQ ID NO: 24)
Right-arm disruptor:
CTTTATTATTAACGGATGCCAAAAGCACACTTCCCATACAACATGGAGAGGCGGG
CGCCC (SEQ ID NO: 25)
PTTG1IP
15-mer RNA: p-CUGCUUUΨUUCUGAA (SEQ ID NO: 26)
Left-arm gBlock:
TAATACGACTCACTATAGGGGGATGCGTTCCATTTTCACACTTCACAGTCATGAAA
ACTGAGAAGACTGTCTTCAGCGTGAACTAAAGTTCACAGGCAGATCACTGATCCA
GAACACTTCAAGAACTCGTCAAACAGCTCGATAAGCCTTTTTGACTGTGTACATCT
GTACCGGGAATAACATTCCTAGGCTGAAATTTCCACAAAGAATAGAACCTGTACC
CAGTTCTTCAGGCTGATTTCCCTGACCTCTTGGGCATTTGTATTTGTAGTAAAGTAT
TGCAGAGATTCCTAAGTATTTTATAGCAGCCATCAAAATTGGACTTTGTATTGTTT
ATTCATAAAAGACACTTGGTAATAGACTTCAGTGAACTCTGTATGAATGCAGTAG
TGTGTGTGCAAAATCCGCTTCCTGAGCGTAGGGTGCTGAGCTGGCGCTAGGGCTC
GGTTGTGAAATACAGCGTAGTCAGCCCTTGCGCTCAGTGTAGAAACCCACGTCTG
TAAGGTCGGTCTTCGTCCATGGGTCGGCATGGCATCTCCACCTCCTCGCGGTCCGA
CCTGGGCTACTTCGGTAGGCTAAGGGAGAAG (SEQ ID NO: 27)
Right-arm gBlock:
TAATACGACTCACTATAGGGATACACTAAGAGCAGCCACAAAACTGTAACCTCAA
GGAAACCATAAAGCTTGGAGTGCCTTAATTTTTAACCAGTTTCCAATAAAACGGTT
TACTACCTGATCGA (SEQ ID NO: 28)
DNA splint:
TTAGTGTATCCCTTCAGAAAAAAGCAGATGGACGAAGAC (SEQ ID NO: 29)
Left-arm disruptor:
GACCTTACAGACGTGGGTTTCTACACTGAGCGCAAGGGCTGACTACGCTGTATTTC
ACAA (SEQ ID NO: 30)
Right-arm disruptor:
TTAAAAATTAAGGCACTCCAAGCTTTATGGTTTCCTTGAGGTTACAGTTTTGTGGC
TGCT (SEQ ID NO: 31)

### Example 3-3: Materials, General Method, and Statistical Analysis

Common security measures for a biosafety level-2 laboratory should be followed. Personal protective attire and chemically resistant gloves, which are important for working with RNA, are required when performing the experiments. All solutions should be made in double-distilled water and sterilized by autoclave or by filtration. The determination of each enzymatic reaction should be performed in triplicate. The results are analyzed by 6% polyacrylamide gel electrophoresis with 7M urea (6% PAGE/7M urea) in denaturing condition or by 1.200 agarose gel in native condition (1.2% agarose). Yields of each reaction are analyzed by image analysis. Statistical difference is evaluated with one-way ANOVA, although users are welcome to employ other suitable software according to the design of each experiment.

### Synthesis of Left-Arm and Right-Arm RNAs by In Vitro Transcription

For assembly of each long RNA containing a site-specific modification, the left-arm and right-arm RNAs are synthesized by in vitro transcription without modification. The assembly of an RNA in the range of 1 kb is described, where the left-arm and right-arm RNAs are in vitro transcribed as ˜500-mers, while the middle RNA is chemically synthesized in the size of a 15-mer, placing the modification at the central position. The templates for in vitro transcription of the left- and right-arm RNAs are made by solid-phase synthesis as double-stranded gBlock DNAs by IDT (Integrated DNA Technologies). For in vitro transcription of the left-arm RNA, the template starts with the consensus T7 promoter sequence, followed by the sequence of interest beginning with three G nucleotides to facilitate transcription, and then by the sequence for the Hepatitis delta virus (HDV) ribozyme. After transcription, the HDV ribozyme self-cleaves to release the left-arm RNA. The HDV ribozyme has the sequence: 5′-GGGUCGGCAUGGCAUCUCCACCUCCUCGCGGUCCGACCUGGGCUACUUCGGUAGG CUAAGGGAGAAG-3′ (SEQ ID NO:1). The DNA template for transcription of the right-arm RNA lacks the 3′-ribozyme sequence but is transcribed in the presence of 20 mM GMP to generate a transcript with a 5′-monophosphate.
1. Double-stranded gBlock DNAs as the templates for in vitro transcription of the left-arm and right-arm RNAs (IDT)
2. HiScribe T7 high yield RNA synthesis kit (New England BioLabs (NEB))
3. RNase-free DNase I (NEB)
4. Monarch RNA Cleanup kit T2040 (NEB)
5. Nanodrop ND-1000 spectrophotometer (ThermoFisher Scientific)
6. Low-range RNA ladder (NEB)
7. Mini-Protean 2-gel vertical electrophoresis system (Bio-Rad)
8. SYBR-Gold staining solution (ThermoFisher Scientific)
9. ChemiDoc imaging system (Bio-Rad)
10. Water baths of different temperatures

### Ribozyme Cleavage of the Left-Arm RNA to Generate a Homogeneous 3′-End

A pair of proximal DNA disruptors (60-mers) is designed to hybridize to the left-arm and right-arm RNAs adjacent to the 3′- and 5′-ends of the DNA splint in each 3-part splint ligation reaction (see FIGS. 13A-13D). These DNA disruptors are synthesized by IDT without purification.
1. Beta-mercaptoethanol (β-Me) (Sigma-Aldrich)
2. Left-arm DNA disruptors (IDT)
3. Right-arm DNA disruptors (IDT)
4. PCR thermocycler (T100 Thermal cycler, Bio-Rad)
5. Low-range RNA ladder (NEB)
6. Mini-Protean 2-gel vertical electrophoresis system (Bio-Rad)
7. SYBR-Gold staining solution (ThermoFisher Scientific)

### Hydrolysis of 2′,3′-Cyclic Phosphate on the Left-Arm RNA

1. T4 PNK (polynucleotide kinase) (NEB)
2. RNase-Out solution (ThermoFisher Scientific)
3. Phenol:chloroform:isoamyl alcohol (25:24:1), pH 5
4. RNase-free water
5. Low-range RNA ladder (NEB)
6. Mini-Protean 2-gel vertical electrophoresis system (Bio-Rad)
7. SYBR-Gold staining solution (ThermoFisher Scientific)

### Three-Part Splint Ligation

1. Left-arm and right-arm RNAs prepared by in vitro transcription
2. Middle RNA with a site-specific W modification in this study (Gene Link)
3. Left-arm DNA disruptor(s) (IDT)
4. Right-arm DNA disruptor(s) (IDT)
5. DNA splint (IDT)
6. Rn12 (NEB)
7. RNase-Out (ThermoFisher Scientific)

### Clean-Up of the 3-Part Splint Ligation Reaction

1. RNA clean and concentrator-5 (Zymo Research)
2. Phenol:chloroform:isoamyl alcohol (25:24:1), pH 5.2
3. Glycogen RNA grade (20 mg/mL) (ThermoFisher Scientific)
4. Deionized formamide (CalBiochem)

### Gel Purification of the Ligated Long RNA for Nanopore Sequencing

1. Agarose gel (1.2%)
2. Owl EasyCast B1A mini horizontal gel electrophoresis system (ThermoFisher Scientific)
3. Purple 6× gel loading dye (NEB)
4. Ethidium bromide (Sigma-Aldrich)
5. Zymoclean gel RNA recovery kit (Zymo Research)
6. Qubit RNA HS assay kit (ThermoFisher Scientific)
7. Agilent RNA 6000 Nano kit (Agilent Technologies)
8. Agilent 2100 Bioanalyzer (Agilent Technologies)
9. Qubit fluorometer 4 (ThermoFisher Scientific)
10. Nanopore Direct RNA sequencing kit (Oxford Nanopore Technologies)
11. MinION Mk1C nanopore sequencer (Oxford Nanopore Technologies)

### Example 3-4: Methods

### Overview: Design of a 3-Part Splint Ligation Scheme to Assemble Long RNA

In some embodiments, a 3-part splint ligation method is provided to synthesize kb-long RNA containing a site-specific internal modification. This method uses in vitro transcription to synthesize the left- and right-arm RNAs, while using chemical synthesis to generate a short middle RNA that contains the modification. Because the left-arm and right-arm RNAs are transcribed from double-stranded gBlock DNAs, which have the capacity to reach 3 kb, this method in principle can assemble an RNA up to, for example, 6 kb long. Additionally, because chemical synthesis can accommodate a wide range of modifications, virtually all naturally occurring modifications in mRNAs can be studied with this method. However, current methods of generating long RNA by 3-part splint ligation have low yields (<2%), due to the 3′-end sequence heterogeneity of in vitro transcribed RNA, and due to the structural heterogeneity of long RNA that can shield the termini from ligation. Here we address these obstacles in a 3-part splint ligation method that improves the yield.

The present example describes the salient features of this method (FIGS. 13A-13D). (i) The left-arm and the right-arm RNAs are synthesized by in vitro transcription in the range of a 500-mer, while the middle RNA is chemically synthesized as a 15-mer with the modification in the center separated from the left- and right-end of ligation by 7 nucleotides each. The length of the 15-mer is chosen to maximize the synthesis yield without purification while providing sufficient sequence for splint ligation. The joining of the three RNAs via a splint ligation leads to a product of ˜1 kb-RNA, which is suitable for nanopore sequencing to determine the sequence accuracy of ligation and to study the basecall properties of the modified base. (ii) For synthesis of the right-arm RNA by in vitro transcription, GMP is added to the NTP mix to promote the incorporation of a 5′-monophosphate, which facilitates ligation. T7 RNAP preferentially initiates RNA synthesis with GMP when it is a component of the reaction mixture. In contrast, for synthesis of the left-arm RNA by in vitro transcription, the 5′-end is less of a concern for ligation and can be made as a 5′-triphosphate. (iii) To minimize the 3′-end sequence heterogeneity, the left-arm RNA is prepared in two steps (FIGS. 13C-13D). It is first transcribed with a 3′-end extension to include the HDV ribozyme, which after synthesis catalyzes self-cleavage to release the left-arm RNA with a 2′,3′-cyclic phosphate at the 3′-end. The cyclic phosphate is then hydrolyzed by T4 PNK to generate a homogeneous 3′-end. The present study chose the HDV ribozyme, which does not have sequence requirements and is broadly applicable to all RNA substrates. (iv) To minimize the conformational heterogeneity of the left- and right-arm RNAs, each is hybridized to a 60-mer DNA proximal disruptor that extends the DNA-RNA hybrid formed in the presence of the DNA splint (FIGS. 13C-13D). When used at a 10-fold molar excess of the RNA, 60-mer DNA proximal disruptors have been shown to promote 3-part ligation by making the termini of the left-arm and right-arm RNAs accessible to ligase. Although a long DNA splint can be used in place of disruptors, a splint cannot be used at high concentration to drive the hybridization reaction with RNA. Ligation products are analyzed by a denaturing PAGE (6%), while the full-length 1 kb RNA is extracted from an agarose gel (1.2%) and purified by a Zymo cartridge, which is more straightforward than electro-elution as described in a recent method.

The present example focused on the RNA modification pseudouridine (ψ) as an example, which is one of the most abundant post-transcriptional modifications in the human transcriptome with a frequency of 0.2-0.6% of total uridines. RNA modifications with ψ confer resistance to degradation and modulate cellular activities of immunogenicity and translation. While ψ has been detected by chemical labeling and Illumina sequencing, different labeling methods identify different sites with limited overlap. In contrast, nanopore sequencing has consistently reported it as a U-to-C mismatch. To quantify the potential of the U-to-C mismatch as an indicator for ψ, a 3-part splint ligation method was employed to construct four synthetic mRNAs, each bearing a ψ in its natural sequence context in the human transcriptome. Two of these (PSMB2; chr1:35603333 and MCM5; chr22:35424407) were annotated with a ψ by chemical coupling, while the other two (MRPS14; chr1:175014468 and PRPSAP1; chr17: 76311411) were detected de novo by the U-to-C error in recent nanopore sequencing analysis. These four synthetic RNAs represent a range of sequence contexts to evaluate the method of 3-part splint ligation.

### Synthesis of Left-Arm and Right-Arm RNAs by In Vitro Transcription

1. Left-arm and right-arm gBlocks (500 ng, ˜1.5 pmoles) are transcribed at 37° C., 2 h, in 20 μL using the NEB HiScribe kit. The transcription reaction for the right-arm RNA is supplemented with 20 mM GMP and 10 mM MgCl₂ to promote incorporation of the 5′-monophosphate (5′-p) which is required for subsequent ligation. T7 RNAP preferentially uses GMP to initiate transcription.
2. After transcription, the gBlock DNAs are hydrolyzed by RNase-free DNase I at 37° C., 15 min, and the RNA products are isolated using the NEB 50 μg-scale Monarch RNA Cleanup cartridges. The yield of purified RNA is usually >100 μg.
3. The concentration of each transcribed RNA is determined by A260 (usually in a 1:50 dilution of the stock) and its size distribution is analyzed (usually 1 μL of the 1:50 dilution) in a 6% denaturing PAGE/7M urea gel. The gel is run in 1×TBE (90 mM Tris, pH 8.0, 90 mM boric acid, 2 mM Na₂EDTA) in a Bio-Rad mini-Protean apparatus for 60 min at 200 V at 60° C., along with a low MW (molecular weight)-range RNA ladder.
4. SYBR Gold-stained gels are imaged to determine the fraction of intact RNA in each sample, which is used to adjust the RNA concentration as determined by A260 to more accurately reflect the concentration that would participate in the ligation reaction. Additional assessment of the RNA concentration is obtained by comparing the RNA band intensity to the known amount of a standard RNA in the same gel.

### HDV Self-Cleavage of the Left-Arm RNA to Generate a Homogeneous 3′-End

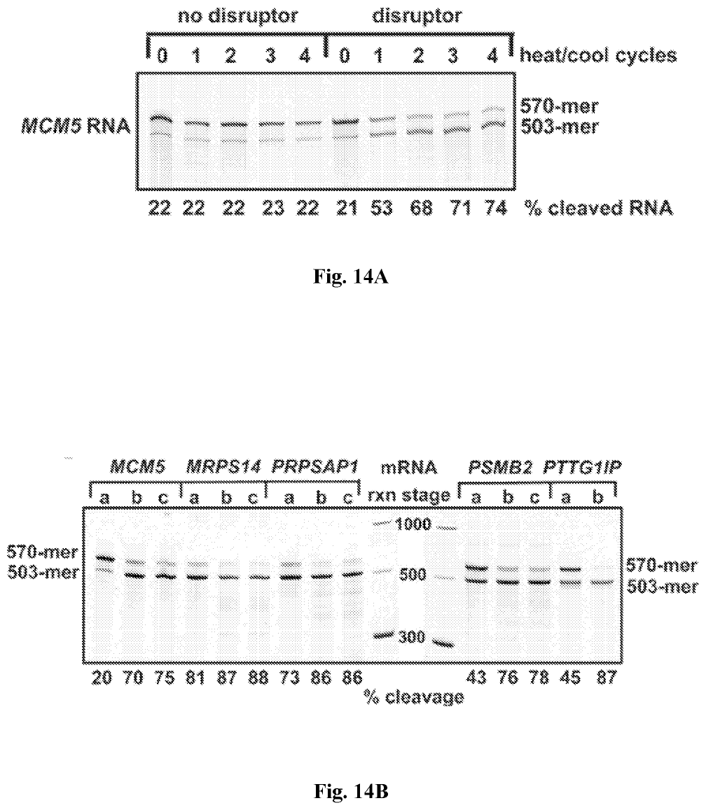

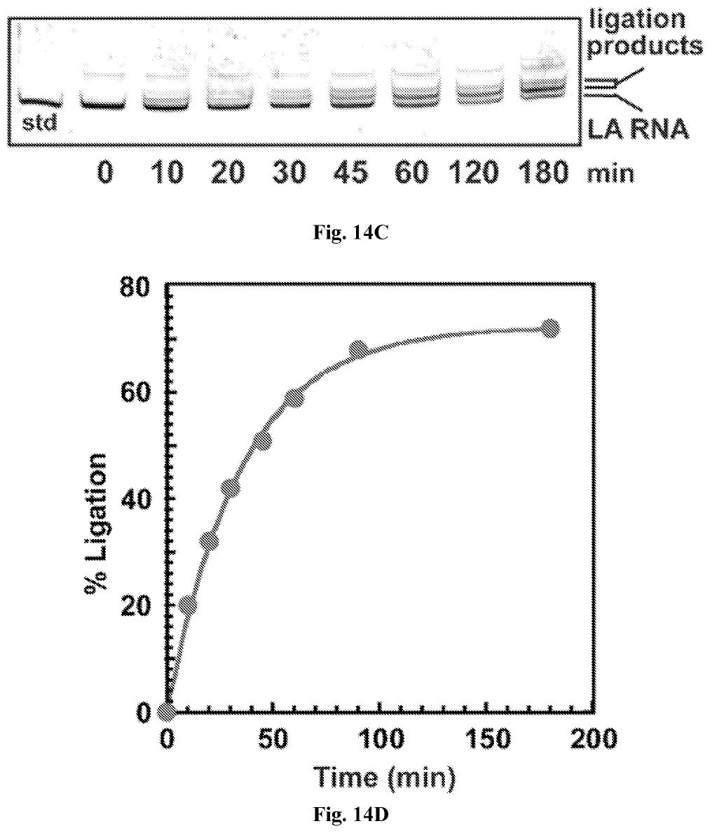

HDV catalyzes self-cleavage to release the transcribed left-arm RNA with a precise 3′-end. It was found that this self-cleavage off a long RNA is most effective in multiple cycles of a heat-cool process and, additionally, in the presence of the left-arm DNA disruptor (FIG. 14A) With the MCM5 RNA as an example, the heat-cool cycling alone did not improve the cleavage yield, whereas cycling in the presence of the disruptor did, increasing the yield from 21 to 74% in the 3rd and 4th cycles. Thus, being an RNA itself with a defined tertiary structure, the HDV ribozyme (67-mer) refolds with the left-arm RNA into the active structure by repeated heat-cool cycles in the presence of the DNA disruptor. This demonstrates the importance of the disruptor to free up the 3′-end of the left-arm RNA for cleavage. Analysis of cleavage of additional left-arm RNAs for MCM5, MRPS14, PRPSAP1, and PSMB2 supports the importance of the disruptor, showing an increased cleavage yield to 70-90% (FIG. 14B).
1. The in vitro transcribed left-arm RNA (200 pmoles) is mixed with a 60-mer left-arm DNA disrupter (2 nmoles) in 90 μL of 110 mM Tris-OAc (Tris-acetate, pH 6.3). The reaction is incubated at 85° C., 2 min, cooled to room temperature, and supplemented with 5 μL 200 mM MgCl₂ and 5 μL 200 mM f-Me to a final volume of 100 μL (at the final concentration of 10 mM MgCl₂, 10 mM Q-Me, 20 μM of the left-arm DNA disruptor, and 2 μM of the left-arm RNA).
2. The reaction is transferred to a PCR tube and incubated in a thermocycler at 72° C., 30s, followed by 4 heat-cool cycles each lasting 15 min between 72° C. and 8° C.
3. The yield of the cleavage is determined by the fraction of the cleaved product in the total input left-arm RNA. The A260 is not informative, due to the presence of disruptor DNA.
   Hydrolysis of 2′,3′-Cyclic Phosphate on the Left-Arm RNA after HDV Cleavage

HDV cleavage produces a 2′,3′-cyclic phosphate at the RNA 3′-end, which needs to be removed before ligation. The present study use the 3′-phosphatase activity of T4 PNK to hydrolyze the cyclic phosphate and to remove the monophosphate. This T4 PNK reaction produces each left-arm RNA in the size as designed (FIG. 14B), indicating that it does not degrade into the body of the RNA. The left-arm RNA after T4 PNK hydrolysis can join with a 15-mer RNA in a 2-part splint ligation, confirming restoration of the terminal 3′-OH (FIG. 14C). The 2-part ligation is efficient, reaching a plateau of 75% in less than 2h (FIG. 14D).
1. Add 1.5 μL of 10 units/L T4 PNK and 1 μL RNase-Out solution (40 units/μL) to the cleavage reaction above.
2. Incubate the hydrolysis reaction at 37° C. for 2 h.
3. Extract the reaction with an equal volume of phenol:chloroform:isoamyl alcohol (25:24:1), pH 5, and precipitate the RNA with 1/10 vol 2.5 M NaOAc, pH 5.0 and 3 vol ethanol.
4. The pellet is washed, air dried, and dissolved in 20 μL RNase-free water.
5. Hydrolysis can be verified by ligation of the T4 PNK-treated left-arm RNA with a 15-mer RNA in a 2-part ligation reaction, using the same splint and conditions as in the 3-part ligation reaction.

### Three-Part Splint Ligation

The present examples provide the protocol below after optimization of several parameters of the 3-part splint ligation reaction.

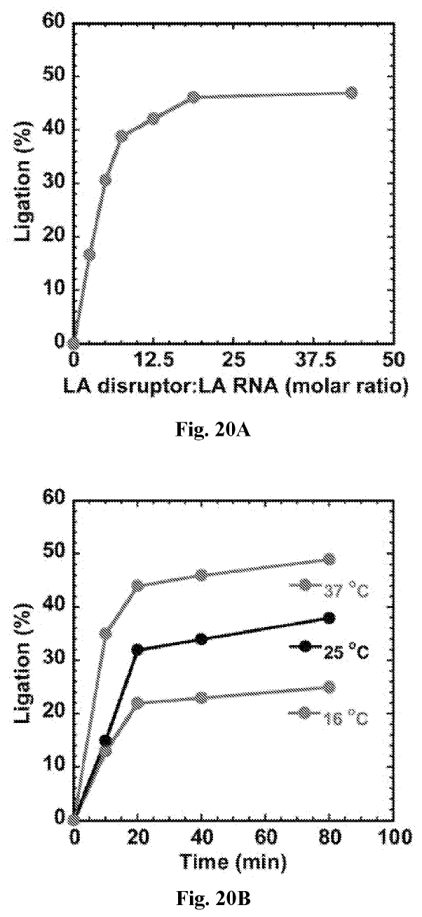

(i) While each DNA disruptor should be in molar excess of its target RNA to drive the ligation reaction, the minimum molar excess should be 20. This was obtained by analysis of a series of titration experiments to monitor the efficiency of 2-part ligation between the left-arm and right-arm RNAs for PSMB2. It was showed that ligation is dependent on the presence of the disruptor, and that the efficiency of ligation increases as a function of the disruptor concentration until it reaches a plateau of 50% at the molar ratio of 18-20 (FIG. 20A). This molar ratio may vary with the length of the target RNA. In an example of joining a 500-mer left-arm RNA with a 500-mer right-arm RNA, the present example used 10 μM of each disruptor to 0.4 μM of the target RNA in a molar ratio of 25, more than sufficient to reach the plateau of ligation efficiency.

(ii) Both the temperature and time influence the ligation efficiency. This was observed with the PSMB2 mRNA in a 2-part splint ligation as above (FIG. 20B). Notably, the ligation efficiency progressively increased with increasing temperature from 16 to 25 to 37° C., indicating that higher temperatures help to unwind internal structures of long RNAs to facilitate ligation. At each temperature, the ligation efficiency reached a plateau at ˜20 min, which appeared to be the time required to assemble the active pre-ligation complex. The consistency of the time across all three temperatures indicates that the efficiency of ligation is rate-limited by the same reaction in different temperatures, which is formation of the active pre-ligation complex, but that once this pre-ligation complex is formed, the actual catalysis by T4 Rnl2 is fast. Indeed, the intrinsic catalytic efficiency of T4 Rnl2 is on the time scale of seconds, much shorter than the 20 min time. Thus, for ligation of long RNAs, temperature is the driving force to form the pre-ligation complex, which is a slow step and is followed by a fast step of catalysis. The identified time of 20 min is shorter than the commonly recommended time (>1h) of splint ligation, providing the benefit of reducing RNA degradation and streamlining long and multistep protocols.

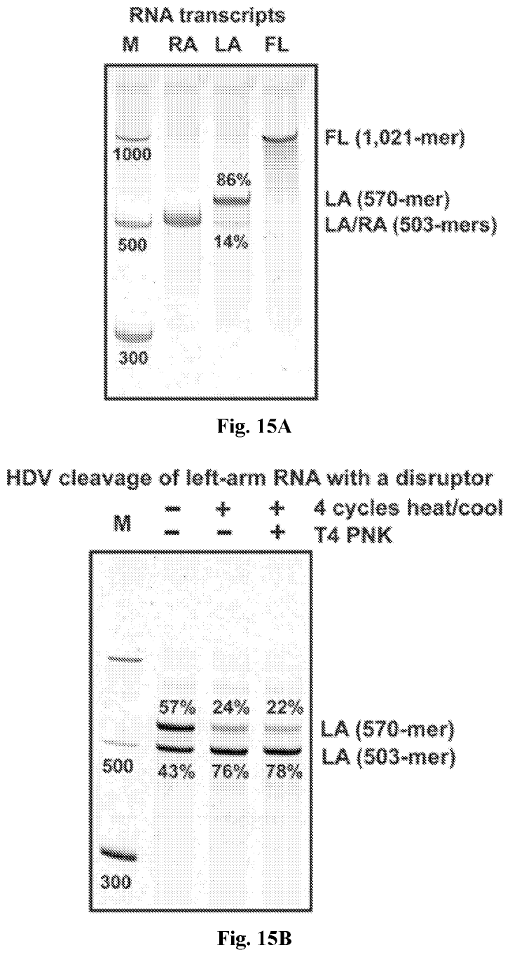

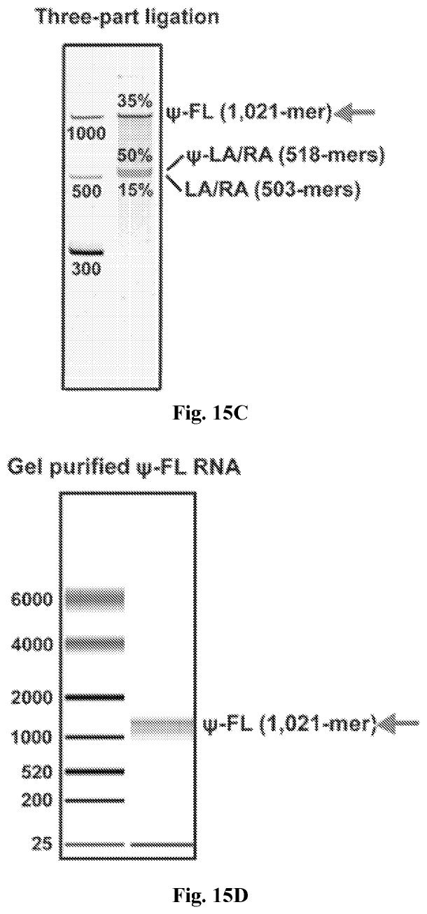

(iii) While multiple components contribute to the ligation efficiency, the major determinant is a pair of DNA disruptors proximal to the two ligation junctions. This was observed by analysis of the contribution of individual components to 3-part ligation for synthesis of a 1 kb PSMB2 RNA (FIGS. 15A-15C, lanes a-c). Notably, having one pair of disruptors proximal to the ligation sites generates 45% yield of ligation (FIGS. 15A-15C, lane c), while lack of any disruptors reduces the yield to less than 10% (FIGS. 15A-15C, lane b), although the addition of an extra pair of disruptors more distal from the ligation sites does not significantly improve the yield (FIGS. 15A-15C, lane a). Indeed, having the distal pair of disruptors alone produces a lower ligation yield at 20% (FIGS. 15A-15C, lane d). Additionally, while processing of the 3′-end of the left-arm RNA by HDV cleavage and by T4 PNK hydrolysis was useful for ligation, the lack of processing still produced a high yield of ligation at 40% (FIGS. 15A-15C, lanes c and e), indicating that the transcribed left-arm RNA did not have a high degree of 3′-end heterogeneity. The issue of 3′-end heterogeneity, however, is likely sequence dependent and may be a barrier in other sequences. Finally, the overall yield of 3-part ligation is similar to that of 2-part ligation (FIGS. 15A-15C, lanes a and f), indicating that the optimized 3-part ligation has yields approaching that of 2-part ligation, the latter of which is the simplest model of ligation.

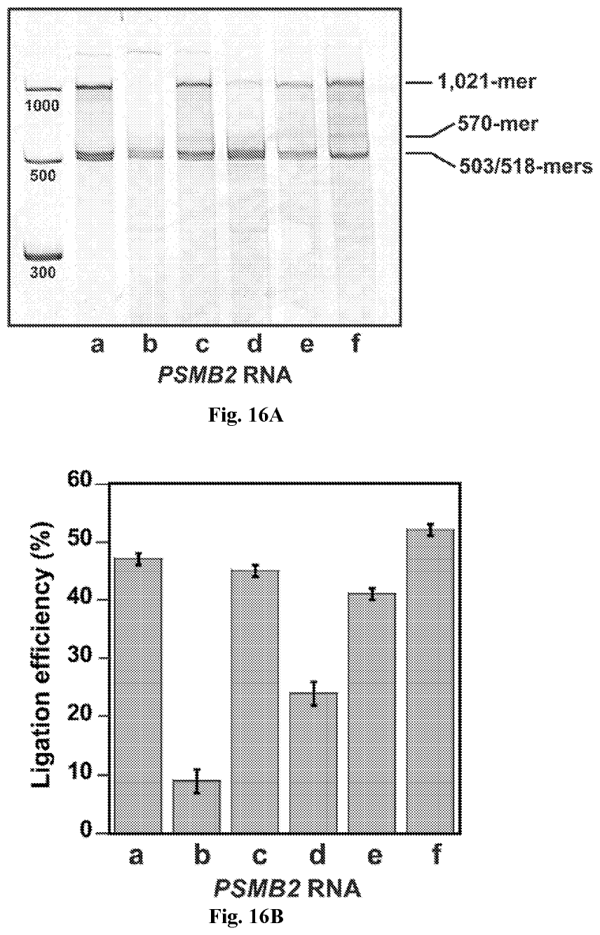

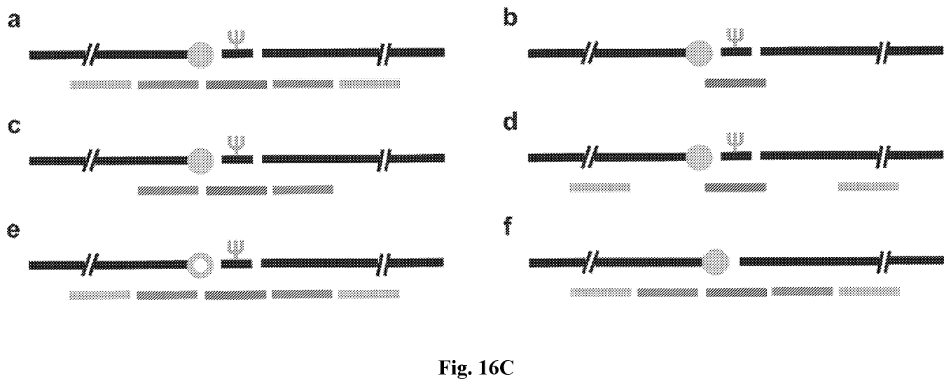

Based on the optimized condition (FIGS. 15A-15C, lane c), the present study provides a step-by-step assembly of a 1 kb PSMB2 RNA containing a V at its natural sequence context (FIGS. 16A-16D). In this example, the left-arm RNA was generated by in vitro transcription with the HDV ribozyme to ensure 3′-end homogeneity. Notably, while the right-arm RNA was also generated by in vitro transcription, it migrated as a homogeneous 503-mer (the transcribed length), whereas the left-arm RNA distributed between a 570-mer (the transcribed length, 86%) and a 503-mer (the HDV-cleaved length, 14%) (FIG. 16A). Thus, a low level of HDV cleavage occurred during in vitro transcription. This cleavage was enhanced upon repeated heat-cool cycles in the presence of the left-arm disruptor, generating 78% of the cleaved left-arm RNA whose 2′,3′-cyclic phosphate at the 3′-end was then removed by T4 PNK (FIG. 16B). The 3′-end processed left-arm RNA was ligated with the right-arm RNA, together with a 15-mer Y-containing synthetic RNA, in a 3-part splint ligation. Analysis of a 6% denaturing PAGE showed 35% as the 3-part ligation product of 1 kb ψ-containing RNA, 50% as the 2-part 518-mer ligation products, representing a mixture of the left-arm and right-arm RNAs each ligated to the 15-mer, and 15% as the mixture of the un-ligated left-arm and right-arm 503-mer RNAs (FIG. 16C). Notably, the yield of the 1 kb RNA (35%) is higher than the reported yields (7-15%) of a similar length RNA generated by conventional 3-part splint ligations (Hertler et al., Nucleic Acids Res. 2022; Zhovmer & Qu, RNA Biol, 13(7), 613-621, 2016).
1. A typical 3-part ligation reaction consists of 1:1:2 molar ratio of the left-arm RNA, the right-arm RNA, and the 15-mer RNA that contains a site-specific modification.
2. These RNAs are mixed with a 24:24:0.9 molar ratio of the left-arm DNA disruptor (60-mer), the right-arm DNA disruptor (60-mer), and the DNA splint (39-mer).
3. For the reactions described here, the molar ratios represent 15 pmoles of the ribozyme-cleaved and T4 PNK-treated left-arm RNA, 15 pmoles of the right-arm RNA, 30 pmoles of the 15-mer RNA with a modification, 360 pmoles of the left-arm DNA disruptor, 360 pmoles of the right-arm DNA disruptor, and 13.5 pmoles of the splint DNA.
4. All components of the 3-part splint ligation are mixed in a 1.6 mL microcentrifuge tube according to the molar ratios above with 3.0 μL of 0.5 M HEPES, pH 7.5, in a total volume of 22.5 μL.
5. The 3-part ligation reaction is initiated at 60° C., 5 min, and cycled down to 5° C. by decreasing 5° C. every 2 min. The annealed 3-part pre-ligation complex is mixed with 7.5 μL of a 4× ligation sub-stock to 30 μL and incubated at 37° C. for 90 min.
6. The 4× ligation sub-stock contains 8 mM MgCl₂, 2 mM ATP/Mgm, 4 mM DTT, 30 μM Rn12, and 2 units/μL RNase-Out.
7. Each 3-part splint ligation is performed in 30 μL with the final concentration of 0.50 μM 3′-end processed left-arm RNA, 0.5 μM right-arm RNA, 1.0 μM 15-mer RNA with a site-specific modification, 12 μM each of the left-arm and right-arm disruptors, 0.45 μM splint DNA, 50 mM HEPES, pH 7.5, 2 mM MgCl₂, 0.5 mM ATP/Mg²⁺, 1.0 mM DTT, 7.5 μM Rnl2, and 0.5 units/μL RNase-Out.
8. Once completed, the 3-part ligation reaction is diluted 4-fold to 120 μL with RNase-free water, supplemented with 12 μL 2.5 M NaOAc, pH 5.0, and 1 μL 20 μg/μL glycogen, and extracted with an equal volume of phenol:chloroform:isoamyl alcohol (25:24:1), pH 5.2. It is then ethanol precipitated, and the pellet dissolved in 15 μL RNase-free water or 70% deionized formamide.
9. In lieu of ethanol precipitation, RNAs>200nts in length can be recovered using a Zymo RNA Clean & Concentrator-5 cartridge as described in the kit.
10. To determine the efficiency of the 3-part ligation, an aliquot of 0.6 μL is run on a 6% denaturing PAGE and visualized by SYBR-Gold staining.

### Gel Purification of the Ligated 1 Kb RNA for Nanopore Sequencing

The improved ligation yields afford purification of the 1 kb W-containing RNA from other RNAs of the ligation reaction. While the 1 kb RNA migrates to a distinct position in a denaturing PAGE, the present study recovered little from the gel by extraction, consistent with the notion that RNA of >600-mer is difficult to extract from denaturing PAGE. Instead, the present study was able to recover 40-50% of the 1 kb RNA from an agarose gel and further purify it by a Zymo cartridge, leading to a product that exhibits a single band on an Agilent Bioanalyzer gel (FIG. 16D). This extraction-purification method is suitable for long RNA.
1. Ligation workups in 70% formamide from the previous step are supplemented with 3 μL of 6× purple gel loading dye, heat denatured at 85° C. for 1 min, and electrophoresed at 100 V, 1 h, in an 1.2% agarose gel (8×7 cm) with 6 wells in TAE buffer (40 mM Tris-acetate, pH 8.3, 1 mM EDTA). An authentic 1 kb RNA standard is included as a reference.
2. Prior to electrophoresis, an optional treatment with DNase I will ensure removal of residual DNA disruptors.
3. The ethidium bromide-stained gel is visualized on a Bio-Rad ChemiDoc imaging system, and a paper printout of the image is used as a guide to excise the 1 kb band of interest.
4. From the agarose gel, 40-50% of the input 1 kb RNA (usually 160-250 ng) is recovered intact in 15 μL water using the Zymoclean gel RNA recovery kit.
5. Concentration of the RNA is determined using the Qubit RNA HS assay kit and its integrity is assessed by the Agilent 2100 Bioanalyzer with an RNA Nanoreagent chip.

### Example 3-5: Design of a 3-Part Splint Ligation Scheme to Assemble Long RNA

The present study, in accordance with some non-limiting embodiments, chose the 3-part splint ligation as a practical method to synthesize kb-long RNA containing a site-specific internal modification. This method is cost-effective, using in vitro transcription to synthesize the long left- and right-arm RNA, while using chemical synthesis to generate a short RNA that contains the modification. Because the left-arm and right-arm RNA are transcribed from double-stranded gBlock DNAs, which have the capacity to reach 3 kb, this method in principle can assemble an RNA up to 6 kb long. Additionally, because chemical synthesis can accommodate a wide range of modifications, virtually all naturally occurring mRNA modifications can be addressed. However, current methods of generating long RNA by 3-part splint ligation have low yields (<2%), due to the 3′-end sequence heterogeneity of in vitro transcribed RNA, and due to the conformational heterogeneity of long RNA that can shield the termini from ligation. Here, the present study addressed these obstacles in a 3-part splint ligation method that improves the yield.

The salient features of the method are described in, for example, FIG. 13A. (i) The left-arm and the right-arm RNA are synthesized by in vitro transcription in the range of a 500-mer, while the middle RNA is chemically synthesized as a 15-mer with the modification in the center separated from the left- and right-end of ligation by 7 nts each. The length of the 15-mer was chosen to maximize the synthesis yield without purification while providing sufficient sequence for splint ligation. The joining of the three RNAs via a splint ligation leads to a product of ˜1 kb-RNA, which is suitable for nanopore sequencing to determine the sequence accuracy of ligation and to study the basecalling properties of the modified base. (ii) To facilitate ligation, the right-arm RNA is synthesized with a 5′-p by adding GMP into the NTP mix of in vitro transcription. T7 RNAP preferentially initiates RNA synthesis with GMP when it is a component of the reaction mixture. The 5′-end of the left-arm RNA is less of a concern for ligation and can be made as a 5′-triphosphate. (iii) To minimize the 3′-end sequence heterogeneity, the left-arm RNA is prepared in two steps (FIG. 13B). It is first transcribed with a 3′-end extension to include the HDV ribozyme, which after synthesis catalyzes self-cleavage to release the left-arm RNA with a 2′,3′-cyclic phosphate at the 3′-end. The cyclic phosphate is then hydrolyzed by T4 PNK to generate a homogeneous 3′-end. The present study chose the HDV ribozyme, which does not have sequence requirements and is broadly applicable to all RNA substrates (although other self-cleaving ribozyme sequences can also be used in some embodiments). (iv) To minimize the conformational heterogeneity of the left- and right-arm RNA, each is provided with a 60-mer DNA disruptor with a complementary sequence that can hybridize adjacent to the left- and right-end of ligation. The length of the disruptor at 60-mer promotes assembly of long RNA by 3-part ligation. The hybrids are designed to make the termini of the left-arm and right-arm RNA accessible to ligation. While a long DNA splint can also be used, synthesis of DNA of >100 nts is more expensive, while the ligation yield decreases. (v) The ligation reaction is analyzed by a denaturing PAGE, while the full-length 1 kb RNA is extracted from an agarose gel and purified by a Zymo cartridge, which is much easier for long RNA than electro-elution.

The present study focused on the RNA modification ψ, which is one of the most abundant post-transcriptional modifications in human transcriptome with a frequency of 0.2-0.6% of total uridines. RNA modifications with ψ confer resistance to degradation and modulate cellular activities of immunogenicity and translation. While ψ has been detected by chemical labeling and next-generation sequencing, different labeling methods identify different sites with limited overlap. Nanopore sequencing instead has consistently reported it as a U-to-C basecalling mismatch. To quantify the potential of the U-to-C mismatch as an indicator for ψ, the present study used the 3-part splint ligation method to construct 5 synthetic mRNAs, each bearing a ψ in its natural sequence context in the human transcriptome. Two of these (PSMB2; chr1:35603333 and MCM5; chr22:35424407) were annotated with a ψ by previous methods, while the other three (MRPS14 chr1:175014468, PRPSAP1; chr17: 76311411, and PTTG1P; chr21:44849705) were detected de novo by the U-to-C error in a nanopore sequencing analysis. These 5 synthetic RNAs represent a range of sequence contexts to evaluate the method of 3-part splint ligation.

### Example 3-6: HDV Cleavage of the Left-Arm RNA

HDV catalyzes self-cleavage to release the transcribed left-arm RNA with a precise 3′-end. This self-cleavage with long RNA is most effective in multiple cycles of a heat-cool process and, unexpectedly, in the presence of the left-arm DNA disruptor (FIG. 14A). With the MCM5 RNA as an example, the heat-cool cycling alone did not improve the cleavage yield, whereas cycling in the presence of the disruptor did, increasing the yield from 21 to 74% in the 3^{rd} and 4^{th} cycles. Thus, as an RNA itself of a defined tertiary structure, the HDV ribozyme (67-mer) refolds with the left-arm RNA most efficiently into the active structure by repeated heat-cool cycles in the presence of the DNA disruptor. This demonstrates the importance of the disruptor to free up the 3′-end of the left-arm RNA for cleavage. Analysis of cleavage of additional left-arm RNAs for MCM5, MRPS14, PRPSAP1, PSMB2, and PTTG1P supports the importance of the disruptor, showing an increased cleavage yield to 70-90% (FIG. 14B).

HDV cleavage produces a 2′,3′-cyclic phosphate at the RNA 3′-end, which needs to be removed before ligation. The present study used the 3′-phosphatase activity of T4 PNK to hydrolyze the cyclic phosphate and to remove the monophosphate. This T4 PNK reaction did not alter the overall size of each left-arm RNA (FIG. 14B), supporting the notion that it is limited to the terminal ribose. The left-arm RNA after T4 PNK hydrolysis was able to join with a 15-mer RNA in a 2-part splint ligation, confirming restoration of the terminal 3′-OH (FIG. 14C). In less than 2 h, the ligation efficiency reached a plateau at 70% (FIG. 14D), which is the maximum efficiency of 2-part ligation (FIG. 16A). This indicates that T4 PNK hydrolysis of the cyclic phosphate was stochiometric and completed in 2 h.

### Example 3-7: Optimization of Splint Ligation

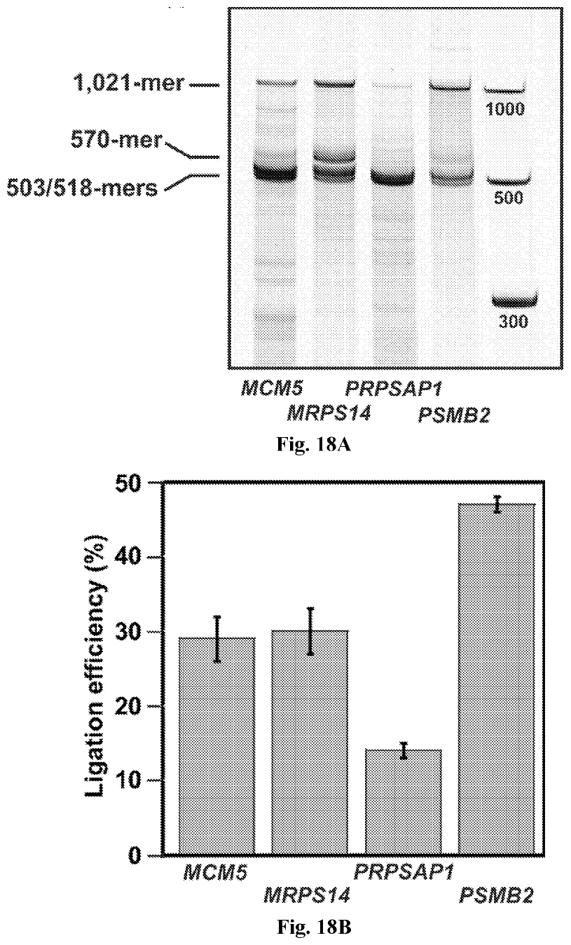

The present study optimized two parameters of the splint ligation reaction. The first is the concentration of the DNA disruptor relative to its complementary RNA, which can strongly influence the efficiency of an inter-molecular ligation reaction. Using the PSMB2 mRNA as an example, the present study designed a 2-part splint ligation reaction, in which the left- and right-arm RNA, each hybridized to a DNA disrupter, were aligned on a 24-mer DNA splint. The present study monitored the ligation efficiency as a function of the concentration of each disruptor relative to its complementary RNA (FIG. 18A). Specifically, the left- and right-arm disruptor were mixed in equal concentration, the left- and right-arm RNA were mixed in equal concentration, while the molar ratio of each disruptor to its RNA varied. Analysis of the molar ratio of the left-arm disruptor to the left-arm RNA as an indicator revealed no ligation in the absence of the disruptor, supporting the importance of the disruptor for ligation of long RNAs. In contrast, increasing concentration of the disruptor increased the ligation efficiency until the molar ratio reached ˜18.0 at the start of a plateau. This molar ratio could vary with the length of the long RNA. In an example of joining a 500-mer left-arm RNA with a 500-mer right-arm RNA, 10 μM of each disruptor was used to 0.4 μM of the RNA in a molar ratio of 25, which is more than sufficient to reach the plateau of ligation efficiency.

The second parameter is the temperature and time of splint ligation. Given the conformational heterogeneity of long RNAs, the accessibility of each to hybridize to the disruptor may be discriminated. RNA secondary and tertiary structures have been proposed as a main contributor to ligation bias. Using the PSMB2 mRNA in a 2-part splint ligation as above (FIG. 18B), the present study observed a progressive increase in the ligation efficiency with increasing temperature from 16 to 25 to 37° C., indicating that higher temperatures help to unwind internal structures of long RNAs to facilitate ligation. At each temperature, we observed a plateau of the ligation efficiency starting at ˜20 min, indicating that this is the time required to assemble the active pre-ligation complex. The consistency of the time across all three temperatures indicates that, once the temperature-dependent formation of the active pre-ligation complex is established, T4 RNL2 readily catalyzes ligation. Indeed, the intrinsic catalytic efficiency of T4 RNL2 is on the time scale of seconds. Thus, for ligation of long RNAs, temperature is the driving force to form the pre-ligation complex, which is a slow step and is followed by a fast step that catalyzes ligation. The identified time of 20 min is shorter than the commonly recommended time (>1h) of splint ligation. The shorter time provides an option to reduce RNA degradation during a longer incubation time. Notably, the present study also observed a slow and gradual increase of ligation efficiency over a time scale of hours (not shown), indicating the possibility of rearrangement of the left-arm and right-arm RNA to make additional ends accessible for ligation.

### Example 3-8: Assembly and Purification of 1 kb RNA Containing a Site-Specific Internal ψ

The present study, in some embodiments, provides a step-by-step procedure to assemble a 1 kb-long RNA containing ψ at its natural sequence context. Using PSMB2 as an example, it was shown that while the left-arm and right-arm RNA were both generated by in vitro transcription, and while the right-arm RNA migrated as a homogeneous 503-mer (the transcribed length), the left-arm RNA displayed a distribution between 86% as a 570-mer (the transcribed length) and 14% as a 503-mer (the HDV-cleaved length) (FIG. 15A). Thus, a low level of the HDV cleavage reaction had occurred during in vitro transcription. This cleavage reaction was further activated upon repeated heat-cool cycles in the presence of the left-arm disruptor, generating 78% of the cleaved left-arm RNA whose 2′,3′-cyclic phosphate at the 3′-end was then removed by T4 PNK (FIG. 15B) The 3′-end processed left-arm RNA was ligated with the right-arm RNA, together with the 15-mer ψ-containing synthetic RNA, in a 3-part splint ligation. Analysis of a 6% denaturing PAGE (FIG. 15C) allowed quantification of each ligation product as the fraction of the input RNA. The present study found 35% as the 3-part ligation product of 1 kb ψ-containing RNA, 50% as the 2-part 518-mer ligation products, representing a mixture of the left-arm and right-arm RNA each ligated to the 15-mer, and 15% as a mixture of the un-ligated left-arm and right-arm 503-mer RNA. Notably, the yield of the 1 kb RNA (35%) is 3-5-fold higher than the reported yields (7-15%) of RNA of similar length generated by a 3-part splint ligation that included disruptors or a long splint but lacked ribozyme-processing of the left-arm RNA. Thus, improving the 3′-end homogeneity of the left-arm RNA is the major determinant of the higher yield.

The improved ligation yields afforded purification of the 1 kb ψ-containing RNA from other RNAs of the ligation reaction. While the 1 kb RNA migrated to a distinct position in a denaturing PAGE, the present study recovered little from the gel by extraction, consistent with the notion that RNA of >600 nts is difficult to extract from denaturing PAGE. Instead, it was found that the 1 kb RNA, which also migrated to a distinct position in an agarose gel, can be extracted with a yield of 40-50% and purified by a Zymo cartridge, leading to a product that showed a single band on an Agilent Bioanalyzer gel (FIG. 15D). This extraction-purification method is more suitable for long RNA than multiple rounds of electroelution, which is prone to degradation of RNA.

### Example 3-9: Ligation Efficiency Dependent on the Length and Sequence Context

The present study quantified the ligation efficiency by denaturing PAGE. Among different sequences of synthetic RNAs, the efficiencies of 3-part ligation to generate the 1 kb RNA ranged in 10-35%, while that of 2-part ligation to generate a mixture of the 518-mer RNAs (e.g., FIG. 15C) ranged in 35-53% (FIG. 16A). Thus, the efficiency varies depending on the sequence context in both the 3-part and 2-part ligation reactions. For each RNA, however, the efficiency is consistently higher in 2-part ligation than in 3-part ligation, although the difference between the two also varies depending on the sequence context. These results emphasize the importance of the sequence context in ligation efficiency.

Several synthetic ψ-containing MCM5 RNAs of different lengths were prepared by 3-part ligation. As the length decreased from 1,021- to 500- and to 300-mer, the yield of the fully ligated product increased from 10 to 14 to 18%. This trend is consistent with the notion that shorter length decreases conformational heterogeneity of the RNA to facilitate ligation. Analysis of the gel-purified RNAs on an Agilent Bioanalyzer (FIG. 16B) showed homogeneity of the 1 kb and 500-mer RNAs as purified from an agarose gel. While this was not the case for the 300-mer (last lane), we found that switching to PAGE purification afforded purification to homogeneity for this shorter length (not shown).

### Example 3-10: Sequencing Analysis Across Ligation Junctions

Errors in 3-part splint ligation cannot be excluded, given the heterogeneity issues of kb-long RNA. Although kb-long RNAs have been evaluated for fidelity as a template for cellular protein synthesis, this is an ensemble analysis that does not determine the fraction of the template that is assembled correctly or incorrectly. At the single-molecule level, while Illumina sequencing was used to determine the sequence accuracy of ligation in a 3-part splint reaction, the accuracy was only examined for the left-junction, but not for the right-junction. It was found that a small fraction of the ligated sequence at the left junction had incorrect nucleotides. These issues could be due to the production of short reads by Illumina sequencing. The present study therefore used nanopore sequencing to report long reads across both ligation junctions in each kb-long RNA generated by our 3-part splint ligation.

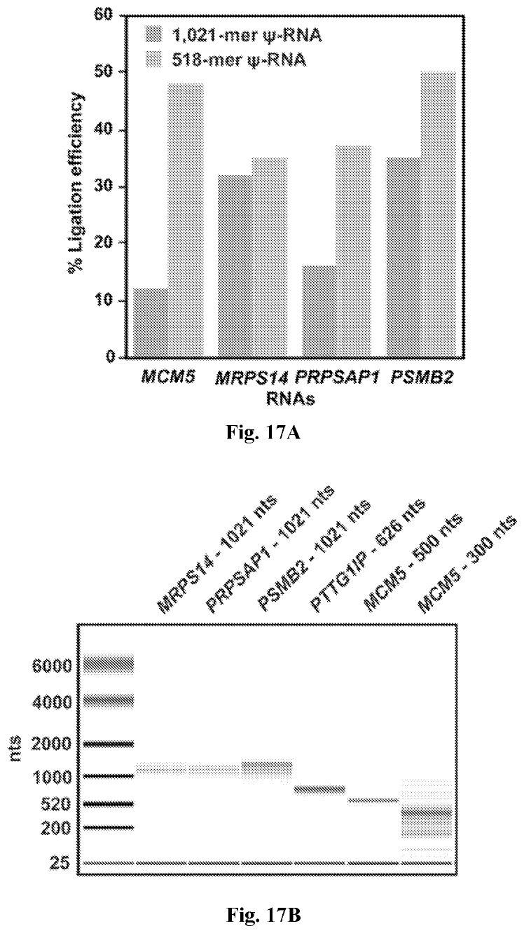

The results showed that, across all 5 ψ-mRNAs, the sequence at each ligation junction located 7 nts from either site of the modification, is homogeneous and accurate (FIGS. 17A-17E). The lack of even a trace of mismatches across ligation of distinct sequence contexts among the 5 ψ-mRNAs is remarkable. Thus, the splint ligation in our optimized condition ensured ligation in the correct order and sequence at both the 5′- and 3′-end. Additionally, the present study observed the U-to-C mismatch corresponding to the ψ in each mRNA, supporting the notion that nanopore reads the modification as a basecalling error. The varying frequencies of the U-to-C mismatch among the 5 ψ-mRNAs indicate differences in the nanopore detection of each modification in different sequence contexts. Notably, we also detected other mismatches adjacent to the ψ in some of these mRNAs, which were not present in the respective IVT control, indicating that they are errors specific to nanopore sequencing. Most likely, they are errors induced by the presence of ψ. Overall, this work demonstrates that our 3-part splint ligation produces kb-long RNA with precise sequence at each ligation junction, providing confidence for subsequent investigation of the ligated RNA

### Example 3-11

Synthesis of kb-long RNA by 3-part splint ligation has historically produced low yields (<2%). Inclusion of DNA disruptors proximal to the ligation sites, or using a long splint DNA, has increased the yield (to 5-15%). The method herein achieved a further improvement of the yield (to 10-35%) by using a cis-acting HDV ribozyme to generate the left-arm RNA with a homogeneous 3′-end in the presence of DNA disruptors. The present study found that the yield varies with the RNA length and sequence context, suggesting that optimization of each ligation is necessary. In some cases, it was found that increasing the splint from a 39-mer to a 50-mer improves the yield. While increasing the molar ratio of the disruptors was previously reported to improve the yield, the present study found no improvement in the examples here. Nor did the present study find improvement in the examples by varying the heat-cool temperatures for ribozyme cleavage prior to ligation, although the present study did find improvement by elevating the temperature of the ligation reaction itself. Thus, each sequence and length for ligation is distinct, and that several factors should be considered to achieve the maximum yield of ligation. These factors include the length of the DNA splint, molar ratio of disruptors to the left- and right-arm RNAs, and temperatures for the ribozyme cleavage and for the RNL2-catalyzed ligation.

A major step forward of this work is the ability to purify kb-long RNA with reasonable yields for nanopore sequencing analysis of the full-length RNA. Notably, RNA longer than hundreds of nucleotides is extremely difficult to isolate and purify, usually appearing as a smear in denaturing PAGE. This is due to the propensity of long RNA to degradation and to the inherent structural heterogeneity that leads to a population of transient isoforms that change continuously and dynamically in gel analysis. Importantly, the present study found that gel extraction, followed by purification through a Zymo cartridge, is a very good method for isolation of kb-long RNA than alternative methods using an affinity tag (see below). Specifically, the present study found that the full-length RNA is most productively extracted from an agarose gel (1.2%), rather than a denaturing PAGE (6%). Because the yield of gel extraction is typically 50% and the yield through a Zymo cartridge is nearly stoichiometric, the present study used this estimation as a guide to design the amounts of input RNAs in each 3-part ligation reaction.

The present study attempted different approaches to isolate kb-long RNA using an affinity tag but found that none produced the yield as high as extraction from an agarose gel. The approaches herein should be considered by others interested in working with long RNA. (i) The present study prepared the DNA splint with a biotin tag and used it to capture the ligated RNAs by a streptavidin resin. The bound RNAs were released from the resin by heat and analyzed on a denaturing PAGE. While the purity of the full-length RNA increased by 2-fold, only 10% of it was recovered. (ii) The present study tested a two-step affinity-hybridization protocol, where one biotinylated probe was used for the left-arm to purify left arm-containing RNAs (products of both 2-part and 3-part ligation), followed by a second biotinylated probe for the right-arm to purify right arm-containing full-length RNA. The present study recovered only 1-2% of the full-length RNA. (iii) The present study considered adding a poly(rA) tail to the right-arm RNA, which after ligation could be purified by a biotinylated oligo(dT) probe. However, this method would also pull down un-ligated and 2-part ligated right-arm RNA.

The improved yields of 3-part splint ligation, combined with isolation of kb-long RNA, have enabled the present study to verify sequence accuracy across ligation junctions by nanopore sequencing. This demonstrates the ability of our method to generate long RNAs not only as synthetic standards for nanopore sequencing, but also as reagents for broader applications in RNA research and RNA-based therapies. Notably, short RNAs with an internal modification have proven valuable for RNA research to investigate RNA conformational dynamics and protein interacting partners. Long RNAs with a site-specific modification can now be used in cellular protein synthesis to determine the effect of the modification. Indeed, RNA biology frequently involves long RNA, such as excision of an intron (average of 5 kb), folding of rRNA (4-5 kb of the 28S and 1.9 kb of the human 18S), and regulation of gene expression by long non-coding RNAs (1-10 kb). In each case, a long RNA can be synthesized as a probe containing an internal fluorophore, or a pair of fluorophores, that respond to environmental changes and undergo fluorescence resonance energy transfer. It is envisioned that the method herein will pave the way for a better understanding of each of these processes.

### Example 3-12

Assembly of kb-long RNA by 3-part splint ligation has historically produced low yields (<2%). Inclusion of DNA disruptors proximal to the ligation sites, or using a long DNA splint, has increased the yield (to 5-15%) (Hertler et al., Nucleic Acids Res 2022; Zhovmer & Qu, RNA Biol, 13(7), 613-621, 2016). The present study reports here a further improvement of the yield (to 15-45%) by two additional features. One is the use of a pair of proximal disruptors at high concentration in conjunction with a short DNA splint present at low concentration, and the other is the use of a left-arm RNA with a homogeneous 3′-end produced by a cis-acting HDV ribozyme.

Using the optimized protocol to generate a 1 kb RNA, we find that the efficiency can vary by 3-fold (FIGS. 18A-18B), depending on the sequence context. This suggests that further optimization for each reaction may be beneficial. Factors to consider include the length of the DNA splint, the number of disruptors, the molar ratio of individual components, and the temperature to form the pre-ligation complex.

While inclusion of a pair of proximal DNA disruptors is clearly important to increase the ligation yield, these disruptors in principle can be replaced by a long DNA splint (˜100-mer). However, the replacement would lose the ability to separately control the molar ratio of the splint and disruptors relative to the left- and right-arm RNAs. The protocol herein has the two disruptors in molar excess of the left- and right-arm RNA by 20-fold to drive hybridization, while limiting the DNA splint to 0.9-fold. If the DNA splint is similarly in molar excess, it would distribute itself to capture the left-arm and the right-arm RNAs separately, reducing the yield of 3-part ligation that requires simultaneous binding of both RNAs.

A major step forward of this work is the ability to purify kb-long RNA with reasonable yields. Notably, RNA longer than hundreds of nucleotides is extremely difficult to isolate and purify. Here the present study found that extraction from an agarose gel, followed by purification through a Zymo cartridge, is the best method for isolation of kb-long RNA. Because the yield of gel extraction is typically 50% with the Zymo kit, the present study used this estimation as a guide to determine the amounts of input RNAs in each 3-part ligation reaction.

Different methods have been attempted to isolate kb-long RNA using affinity tags but that none was found to produce a yield as high as extraction from an agarose gel. The present study describe the attempts for consideration by others interested in working with long RNA. (i) The present study prepared the DNA splint with a biotin tag and used it to capture the ligated RNAs by a streptavidin resin. The bound RNAs were released from the resin by heat and analyzed on a denaturing PAGE. While the purity of the full-length RNA increased by 2-fold, its recovery was only 10%. (ii) The present study tested a two-step affinity-hybridization protocol, using one biotinylated probe for the left-arm to purify left arm-containing RNAs, followed by using a second biotinylated probe for the right-arm to purify right arm-containing full-length RNA. The present study recovered only 1-2% of the starting full-length RNA. (iii) The present study considered adding a poly(rA) tail to the right-arm RNA to allow purification of the ligated RNA by a biotinylated oligo(dT) probe. However, this method would also pull down un-ligated and 2-part ligated right-arm RNA and was not explored.

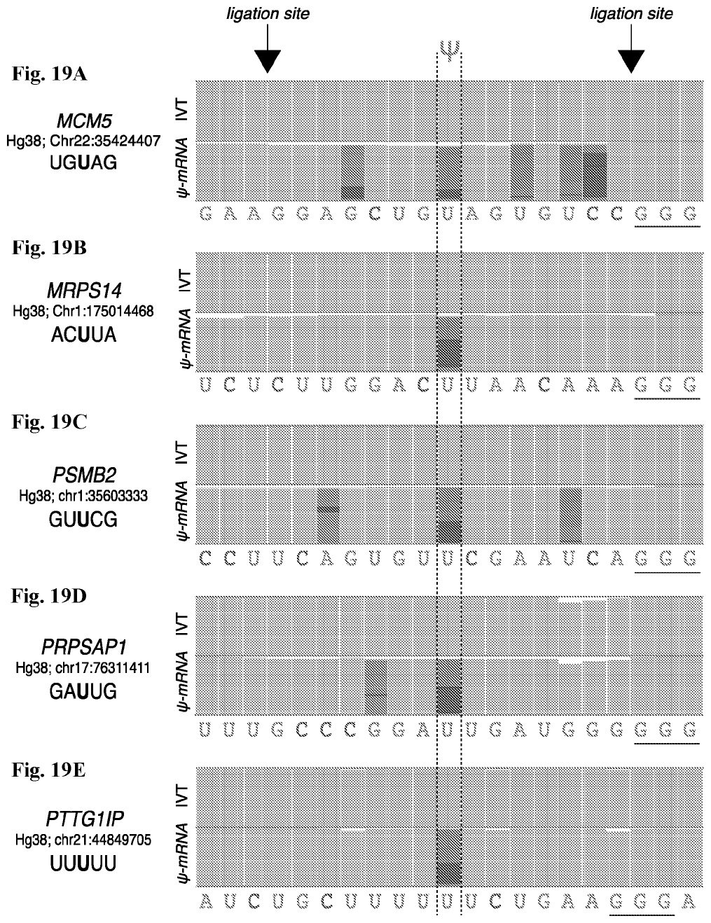

The present study isolated several 1 kb RNAs and verified the sequence accuracy across each ligation junction. Notably, errors in 3-part splint ligation cannot be excluded, given the heterogeneity of kb-long RNA. Previously, kb-long ligated RNAs were assembled using a long splint DNA, instead of DNA disruptors, and were evaluated for fidelity as a template for cellular protein synthesis in an ensemble analysis that did not determine the fraction of correctly ligated RNA (Hertler et al., Nucleic Acids Res 2022). At the single-molecule level, although the assembled kb-long RNAs were evaluated for fidelity by Illumina sequencing, only the left junction of a 3-part ligation reaction was evaluated, which was found to contain a small fraction of incorrect nucleotides (Hertler et al., Nucleic Acids Res. 2022). Here the present study used nanopore sequencing to report long reads across both ligation junctions. Specifically, gel-purified 1 kb RNAs from 3-part ligation, each containing a site-specific xii, were combined for RNA sequencing (SQK-RNA002) using the ONT direct RNA sequencing protocol (DRCE_9080_v2 revH_14Aug2019) (Tavakoli et al., Nat Commun, 14(1), 334, 2023). Across all four kb-long RNAs, the present study found that the sequence at each ligation junction located 7 nucleotides from either site of the modification is homogeneous and accurate (FIGS. 19A-19E). Thus, the optimized 3-part ligation protocol herein correctly assembled a long RNA without a trace of mismatch, providing confidence for subsequent investigation of the ligated RNA. Additionally, the method herein was able to detect the U-to-C mismatch corresponding to the y in each mRNA, supporting the notion that nanopore reads the modification as a basecall error. The varying frequencies of the U-to-C mismatch among the four RNAs indicate differences in the nanopore detection of each modification in different sequence contexts. Notably, the present study also detect other mismatches adjacent to the y in some of these RNAs, which are not present in the respective in vitro transcribed (IVT) control, indicating that they are errors induced by the presence of ψ in nanopore sequencing and pointing to the need for further improvement of the sequencing technology.

The present study demonstrates here the ability of our protocol to generate long RNAs as synthetic standards for mapping and quantification of a modification in nanopore sequencing. More broadly, these long RNAs also serve as powerful tools for RNA research. As shown for the utility of short RNAs with an internal modification for RNA research, long RNAs with an internal modification can now be used to probe reactions such as excision of an intron (average of 5 kb), folding of rRNA (4-5 kb of the 28S and 1.9 kb of the 18S), and regulation of gene expression by non-coding RNAs (1-10 kb). In each case, a long RNA can be assembled as a probe containing an internal fluorophore, or a pair of fluorophores, as the reporters that probe the dynamic changes of the RNA structure. It is envisioned that the method herein will facilitate a better understanding of each of these reactions in the transcriptomes.

### Enumerated Embodiments

In some aspects, the present invention is directed to the following non-limiting embodiments:

Embodiment 1: A method of preparing an RNA molecule present in a composition for sequencing, comprising: contacting the RNA molecule with an RNA-dependent RNA polymerase (RdRp) in the composition, wherein the RdRp extends the 3′ end of the RNA molecule using the RNA molecule as a template.

Embodiment 2: The method of Embodiment 1, wherein the RNA molecule comprises a hairpin structure at the 3′ end.

Embodiment 3: The method of Embodiment 1 or 2, wherein the RdRp is an eukaryotic RdRp, an RdRp from a Birnaviridae family virus, an RdRp from a Bunyaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, or an RdRp from a Reoviridae family virus.

Embodiment 4: The method of any one of Embodiments 1-3, wherein the RdRp is 3D polymerase (3D^{pol}) from a poliovirus.

Embodiment 5: The method of any one of Embodiments 1-4, wherein the composition further comprises a nucleoside triphosphate.

Embodiment 6: The method of any one of Embodiments 1-5, wherein the composition further comprises a magnesium ion (Mg²⁺) or a manganese (II) ion (Mn²⁺).

Embodiment 7: The method of any one of Embodiments 1-6, wherein the RNA molecule is fully extended such that RdRp-driven replication reaches the 5′ end of the RNA molecule.

Embodiment 8: The method of anyone of Embodiments 1-7, wherein the RNA molecule comprises a modified nucleotide, which is optionally pseudouridine.

Embodiment 9: The method of any one of Embodiments 1-8, wherein the length of the RNA molecule is about 1 kilobase (kb) or longer, such as about 1.5 kb or longer, about 2 kb or longer, about 2.5 kb or longer.

Embodiment 10: The method of any one of Embodiments 1-9, further comprising attaching a barcoding sequence to the RNA molecule extended by the RdRp.

Embodiment 11: A method of sequencing an RNA molecule, the method comprising: preparing a first RNA composition using the method according to any one of Embodiments 1-10; and sequencing the RNA molecule extended by the RdRp in the first RNA composition.

Embodiment 12: The method of Embodiment 11, wherein the sequencing the RNA molecule extended by the RdRp comprises a direct RNA sequencing.

Embodiment 13: The method of Embodiment 11 or 12, wherein the sequencing comprises nanopore sequencing.

Embodiment 14: The method of any one of Embodiments 11-13, wherein the RNA molecule comprises a modified nucleotide, which is optionally pseudouridine, and the method further comprises comparing the sequencing results of the native portion of the extended RNA molecule and the sequencing results of extended portion of the extended RNA molecule to identify the modified nucleotide.

Embodiment 15: A kit for preparing an RNA molecule present in a composition for sequencing, comprising: an RNA-dependent RNA polymerase (RdRp) capable of extending a 3′ end of an RNA molecule using the RNA molecule as a template; and a manual instructing that the RNA molecule be contacted with the RdRp before performing the sequencing.

Embodiment 16: The kit of Embodiment 15, wherein the RNA molecule comprises a hairpin structure at the 3′ end.

Embodiment 17: The kit of Embodiment 15 or 16, wherein the RdRp is an eukaryotic RdRp, an RdRp from a Birnaviridae family virus, an RdRp from a Bunyaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, or an RdRp from a Reoviridae family virus.

Embodiment 18: The kit of any one of Embodiments 15-17, wherein the RdRp is 3D polymerase (3D^{pol}) from a poliovirus.

Embodiment 19: The kit of any one of Embodiments 15-18, further comprising a nucleoside triphosphate.

Embodiment 20: The kit of any one of Embodiments 15-19, further comprising a magnesium ion (Mg²⁺) or a manganese (II) ion (Mn²⁺).

Embodiment 21: The kit of any one of Embodiments 16-20, further comprising a barcoding nucleic acid molecule, and an enzyme for attaching the barcoding nucleic acid molecule to the RNA molecule extended by the RdRp.

Embodiment 22: The kit of Embodiment 21, wherein the enzyme for attaching the barcoding nucleic acid molecule to the RNA molecule extended by the RdRp comprises an RNA ligase, optionally a T4 RNA ligase 1, T4 RNA ligase 2, or a derivative thereof.

Embodiment 23: A method of preparing an RNA molecule having a modified nucleic acid, the method comprising: preparing a ligation mixture comprising: a left-arm RNA segment for forming a 5′-portion of the RNA molecule; a middle RNA segment comprising the modified nucleic acid for forming a middle portion of the RNA molecule; a right-arm RNA segment for forming a 3′-portion of the RNA molecule; and a DNA splint molecule complementary to the RNA molecule, wherein the DNA splint molecule overlaps with an entirety of the middle RNA segment, a 3′-end of the left-arm RNA segment, and a 5′-end of the right-arm RNA segment; and ligating the left-arm RNA segment, the middle RNA segment, and the right-arm RNA segment to form the RNA molecule having the modified nucleic acid.

Embodiment 24: The method of Embodiment 23, wherein the method further comprises preparing the left-arm RNA segment by in vitro transcription of a first DNA template.

Embodiment 25: The method of Embodiment 24, wherein the first DNA template encodes a pre-left-arm RNA segment comprising the left-arm RNA segment and a cis-cleaving ribozyme to the 3′-end of the left-arm RNA segment.

Embodiment 26: The method of Embodiment 25, wherein, after the in vitro transcription of the first DNA template, the cis-cleaving ribozyme in the pre-left-arm RNA segment removes itself from the pre-left-arm RNA segment, thereby resulting in a left-arm RNA segment having a homogeneous 3′-end.

Embodiment 27: The method of Embodiment 26, wherein preparing the left-arm RNA segment comprises contacting the pre-left-arm RNA segment with a first DNA disruptor, and allowing the cis-cleaving ribozyme to remove itself from the pre-left-arm RNA segment in the presence of the first DNA disruptor, wherein the first DNA disruptor is a DNA molecule complementary to a 3′-portion of the left-arm RNA segment.

Embodiment 28: The method of Embodiment 26 or 27, wherein preparing the left-arm RNA segment comprises subjecting a mixture comprising the pre-left-arm RNA segment and the first DNA disruptor to one or more cycles of heating and cooling.

Embodiment 29: The method of any one of Embodiments 25-28, wherein the cis-cleaving ribozyme comprises at least one selected from the group consisting of a Hepatitis delta virus (HDV) ribozyme or HDV-like self-cleaving ribozyme, a hammerhead ribozyme, hairpin ribozyme, a Varkud Satellite (VS) ribozyme, a glmS ribozyme, and a twister ribozyme.

Embodiment 30: The method of any one of Embodiments 25-29, wherein preparing the left-arm RNA segment by in vitro transcription of the first DNA template comprises using PNK to enzymatically treat the left-arm RNA segment to form a mature 3′-OH end in the left-arm RNA segment.

Embodiment 31: The method of any one of Embodiments 24-30, wherein preparing the left-arm RNA segment further comprises purifying the left-arm RNA segment from a reaction mixture for preparing the left-arm RNA segment, and wherein purifying the left-arm RNA segment comprises: subjecting the reaction mixture to an agarose gel electrophoresis; isolating an agarose gel section comprising the left-arm RNA segment from the agarose gel; and isolating the left-arm RNA segment from the isolated agarose gel section.

Embodiment 32: The method of any one of Embodiments 23-31, wherein a length of the left-arm RNA segment ranges from about 200 bases to about 3,500 bases.

Embodiment 33: The method of any one of Embodiments 23-32, wherein the middle RNA segment is chemically synthesized.

Embodiment 34: The method of any one of Embodiments 23-33, wherein a length of the middle RNA segment ranges from about 5 bases to about 100 bases.

Embodiment 35: The method of any one of Embodiments 23-34, wherein the modified nucleic acid of the middle RNA segment comprises a modified base, a modified sugar group and/or a modified backbone.

Embodiment 36: The method of any one of Embodiments 23-35, wherein the right-arm RNA segment is prepared from in vitro transcription using a second DNA template.

Embodiment 37: The method of any one of claims 23-36, wherein a length of the right-arm RNA segment ranges from about 200 bases to about 3,500 bases.

Embodiment 38: The method of any one of Embodiments 23-37, wherein the ligation mixture further comprises: a second DNA disruptor complementary with a 3′-portion of the left-arm RNA segment; and a third DNA disruptor complementary with a 5′-portion of the right-arm RNA segment.

Embodiment 39: The method of any one of Embodiments 27-38, wherein the second DNA disruptor and the first DNA disruptor are the same or different.

Embodiment 40: The method of any one of Embodiments 23-39, wherein ligating the left-arm RNA segment, the middle RNA segment, and the right-arm RNA segment comprises subjecting the ligation mixture to an RNA ligase.

Embodiment 41: The method of any one of Embodiments 38-40, wherein a ratio between a molarity of the second DNA disruptor and/or the third DNA disruptor to a molarity of the left-arm RNA segment, the middle RNA segment and/or the right-arm segment is about 10 or larger.

Embodiment 42: The method of any one of Embodiments 23-41, wherein a temperature for ligating the left-arm RNA segment, the middle RNA segment, and the right-arm RNA segment ranges from about 14° C. to about 25° C.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A method of preparing an RNA molecule present in a composition for sequencing, comprising:
contacting the RNA molecule with an RNA-dependent RNA polymerase (RdRp) in the composition, wherein the RdRp extends the 3′ end of the RNA molecule using the RNA molecule as a template.

2. The method of claim 1, wherein at least one of the following applies:
(a) the RNA molecule comprises a hairpin structure at the 3′ end,
(b) wherein the RdRp is an eukaryotic RdRp, an RdRp from a Birnaviridae family virus, an RdRp from a Bunvaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, or an RdRp from a Reoviridae family virus;
(c) the RdRp is 3D polymerase (3D^{pol}) from a poliovirus;
(d) the composition further comprises a nucleoside triphosphate;
(e) the composition further comprises a magnesium ion (Mg²⁺) or a manganese (II) ion (Mn²⁺);
(f) the RNA molecule is fully extended such that RdRp-driven replication reaches the 5′ end of the RNA molecule;
(g) the length of the RNA molecule is about 1 kilobase (kb) or longer, such as about 1.5 kb or longer, about 2 kb or longer, about 2.5 kb or longer; or
(h) the method further comprises attaching a barcoding sequence to the RNA molecule extended by the RdRp.

3-7. (canceled)

8. The method of claim 1, wherein the RNA molecule comprises a modified nucleotide, which is optionally pseudouridine.

9-10. (canceled)

11. A method of sequencing an RNA molecule, the method comprising:
preparing a first RNA composition using the method according to claim 1; and
sequencing the RNA molecule extended by the RdRp in the first RNA composition.

12. The method of claim 11, wherein at least one of the following applies:
(a) the sequencing the RNA molecule extended by the RdRp comprises a direct RNA sequencing;
(b) the sequencing comprises nanopore sequencing.

13. (canceled)

14. The method of claim 11, wherein
the RNA molecule comprises a modified nucleotide, which is optionally pseudouridine, and
the method further comprises comparing the sequencing results of the native portion of the extended RNA molecule and the sequencing results of extended portion of the extended RNA molecule to identify the modified nucleotide.

15. A kit for preparing an RNA molecule present in a composition for sequencing, comprising:
an RNA-dependent RNA polymerase (RdRp) capable of extending a 3′ end of an RNA molecule using the RNA molecule as a template; and
a manual instructing that the RNA molecule be contacted with the RdRp before performing the sequencing.

16. The kit of claim 15, wherein at least one of the following applies:
(a) the RNA molecule comprises a hairpin structure at the 3′ end,
(b) the RdRp is an eukaryotic RdRp, an RdRp from a Birnaviridae family virus, an RdRp from a Bunvaviridae family virus, an RdRp from a Caliciviridae family virus, an RdRp from a Cystoviridae family virus, an RdRp from a Fiersviridae family virus, an RdRp from a Flaviviridae family virus, an RdRp from a Leviviridae family virus, an RdRp from a Permutatetraviridae family virus, an RdRp from a Picornaviridae family virus, or an RdRp from a Reoviridae family virus;
(c) the RdRp is 3D polymerase (3D^{pol}) from a poliovirus;
(d) the kit further comprises a nucleoside triphosphate;
(e) the kit further comprises a magnesium ion (Mg²⁺) or a manganese (II) ion (Mn²⁺); or
(f) the kit further comprises a barcoding nucleic acid molecule, and an enzyme for attaching the barcoding nucleic acid molecule to the RNA molecule extended by the RdRp.

17-20. (canceled)

21. The kit of claim 15, further comprising a barcoding nucleic acid molecule, and an enzyme for attaching the barcoding nucleic acid molecule to the RNA molecule extended by the RdRp.

22. The kit of claim 21, wherein the enzyme for attaching the barcoding nucleic acid molecule to the RNA molecule extended by the RdRp comprises an RNA ligase, optionally a T4 RNA ligase 1, T4 RNA ligase 2, or a derivative thereof.

23. A method of preparing an RNA molecule having a modified nucleic acid, the method comprising:
preparing a ligation mixture comprising:
a left-arm RNA segment for forming a 5′-portion of the RNA molecule;
a middle RNA segment comprising the modified nucleic acid for forming a middle portion of the RNA molecule;
a right-arm RNA segment for forming a 3′-portion of the RNA molecule; and
a DNA splint molecule complementary to the RNA molecule, wherein the DNA splint molecule overlaps with an entirety of the middle RNA segment, a 3′-end of the left-arm RNA segment, and a 5′-end of the right-arm RNA segment; and
ligating the left-arm RNA segment, the middle RNA segment, and the right-arm RNA segment to form the RNA molecule having the modified nucleic acid.

24. The method of claim 23, wherein the method further comprises preparing the left-arm RNA segment by in vitro transcription of a first DNA template.

25. The method of claim 24, wherein the first DNA template encodes a pre-left-arm RNA segment comprising the left-arm RNA segment and a cis-cleaving ribozyme to the 3′-end of the left-arm RNA segment.

26. The method of claim 25, wherein, after the in vitro transcription of the first DNA template, the cis-cleaving ribozyme in the pre-left-arm RNA segment removes itself from the pre-left-arm RNA segment, thereby resulting in a left-arm RNA segment having a homogeneous 3′-end.

27. The method of claim 26, wherein at least one of the following applies:
(a) preparing the left-arm RNA segment comprises contacting the pre-left-arm RNA segment with a first DNA disruptor, and allowing the cis-cleaving ribozyme to remove itself from the pre-left-arm RNA segment in the presence of the first DNA disruptor, and the first DNA disruptor is a DNA molecule complementary to a 3′-portion of the left-arm RNA segment; or
(b) preparing the left-arm RNA segment comprises subjecting a mixture comprising the pre-left-arm RNA segment and the first DNA disruptor to one or more cycles of heating and cooling.

28. (canceled)

29. The method of claim 25, wherein at least one of the following applies:
(a) the cis-cleaving ribozyme comprises at least one selected from the group consisting of a Hepatitis delta virus (HDV) ribozyme or HDV-like self-cleaving ribozyme, a hammerhead ribozyme, hairpin ribozyme, a Varkud Satellite (VS) ribozyme, a glmS ribozyme, and a twister ribozyme; or
(b) preparing the left-arm RNA segment by in vitro transcription of the first DNA template comprises enzymatically treating the left-arm RNA segment to form a mature 3′-OH end in the left-arm RNA segment, optionally the enzymatic treatment of the left-arm RNA segment is with a polynucleotide kinase (PNK).

30. (canceled)

31. The method of claim 24, wherein preparing the left-arm RNA segment further comprises purifying the left-arm RNA segment from a reaction mixture for preparing the left-arm RNA segment, and wherein purifying the left-arm RNA segment comprises:
subjecting the reaction mixture to an agarose gel electrophoresis;
isolating an agarose gel section comprising the left-arm RNA segment from the agarose gel; and
isolating the left-arm RNA segment from the isolated agarose gel section.

32. The method of claim 23, wherein
(a) a length of the left-arm RNA segment ranges from about 200 bases to about 3,500 bases;
(b) the middle RNA segment is chemically synthesized;
(c) a length of the middle RNA segment ranges from about 5 bases to about 100 bases;
(d) the modified nucleic acid of the middle RNA segment comprises a modified base, a modified sugar group or a modified backbone;
(e) the right-arm RNA segment is prepared from in vitro transcription using a second DNA template;
(f) a length of the right-arm RNA segment ranges from about 200 bases to about 3,500 bases;
(g) the ligation mixture further comprises: a second DNA disruptor complementary with a 3′-portion of the left-arm RNA segment; and a third DNA disruptor complementary with a 5′-portion of the right-arm RNA segment;
(h) ligating the left-arm RNA segment, the middle RNA segment, and the right-arm RNA segment comprises subjecting the ligation mixture to an RNA ligase;
(i) a temperature for ligating the left-arm RNA segment, the middle RNA segment, and the right-arm RNA segment ranges from about 14° C. to about 25° C.;
(j) a length of the RNA molecule prepared by the method ranges from about 400 bases to about 6,000 bases;
(k) a yield of RNA molecule based on a molarity of the left-arm RNA segment, the middle RNA segment or the right-arm segment is about 20% or greater: or
(l) the RNA molecule prepared by the method is substantially free of heterogeneity and mismatches around a ligation point between the left-arm RNA segment and the middle RNA segment, and a ligation point between the middle RNA segment and the right-arm RNA segment.

33-37. (canceled)

38. (canceled)

39. The method of claim 27, wherein the second DNA disruptor and the first DNA disruptor are the same or different.

40. (canceled)

41. The method of claim 23, wherein a ratio between a molarity of the second DNA disruptor or the third DNA disruptor to a molarity of the left-arm RNA segment, the middle RNA segment or the right-arm segment is about 10 or larger.

42. (canceled)

43. The method of claim 23 wherein the method further comprises, after the ligation reaction, purifying the RNA molecule from the ligation mixture, and purifying the RNA molecule from the ligation mixture comprises:
subjecting the ligation mixture to an agarose gel electrophoresis;
isolating an agarose gel section from the agarose gel, wherein the agarose gel section comprises the RNA molecule; and
purifying the RNA molecule from the agarose gel section.

44-46. (canceled)
